SEQUENCE LISTING (6 sequences)

SEQ ID NO: 1 - DNA, 801 bp, Homo sapiens
  sig_peptide (1)..(72)
atgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccag

SEQ ID NO: 2 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 3 - protein, 170 aa, Homo sapiens
  PEPTIDE (1)..(170): 18 kDa N-terminal fragment
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDEL

SEQ ID NO: 4 - DNA, 510 bp, Homo sapiens
gatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctg

SEQ ID NO: 5 - protein, 12 aa, Artificial
  Artificial peptide
YGRKKRRRQRRR

SEQ ID NO: 6 - protein, 15 aa, Artificial
  Artificial peptide
GGGGYGRKKRRQRRR

(19) United States
(12) Patent Application Publication
(10) Pub. No.: US 2008/0027000 A1
(43) Pub. Date: Jan. 31, 2008

(54) APO-A-I Regulation of T-Cell Signaling

(76) Inventors: Jean-Michel Dayer, Geneva (CH); Danielle Burger, Carouge (CH); Tadahiko Kohno, Thousand Oaks, CA (US); Carl K. Edwards III, Thousand Oaks, CA (US)

Correspondence Address: FULBRIGHT & JAWORSKI L.L.P., 600 CONGRESS AVE., SUITE 2400, AUSTIN, TX, 78701, US

(21) Appl. No.: 11/774,258

(22) Filed: Jul. 6, 2007

Related U.S. Application Data
(62) Division of application No. 09/803,918, filed on Mar. 13, 2001.
(60) Provisional application No. 60/189,008, filed on Mar. 13, 2000.
(60) Provisional application No. 60/193,551, filed on Mar. 31, 2000.

(51) Int. Cl. A61K 38/16; A61P 29/00

(52) U.S. Cl. 514/12

(57) ABSTRACT

The invention provides AFTI polypeptides and nucleic acid molecules encoding the same. The invention also provides vectors, host cells, selective binding agents, and methods for producing AFTI polypeptides. Also provided are methods for the treatment, diagnosis, amelioration, or prevention of diseases with AFTI polypeptides, particularly IL-1 mediated diseases, TNF-α mediated diseases, and diseases involving monocyte activation.

This application is a divisional of co-pending application U.S. Ser. No. 09/803,918 filed Mar. 13, 2001, and claims the benefit of U.S. Provisional Application No. 60/189,008, filed Mar. 13, 2000 and of U.S. Provisional Application No. 60/193,551, filed Mar. 31, 2000, all of which are hereby incorporated by reference herein in their entirety for any purpose.

### FIELD OF THE INVENTION

The present invention relates to apolipoprotein A-1 (apo-A-1) and fragments and derivatives thereof and their use in regulating T-cell-mediated activation of monocytes. The invention also relates to vectors, host cells, pharmaceutical compositions, selective binding agents and methods for producing such apo-A-1 related molecules. Also provided are methods for the diagnosis, treatment, amelioration, and/or prevention of diseases associated with T-cell-mediated activation of monocytes.

### BACKGROUND AND SUMMARY OF THE INVENTION

The importance of tumor necrosis factor-ct (TNF-α) and interleukin-1 (IL-1) in chronic inflammation has been well established. The blockade or inhibition of these proinflammatory cytokines in vivo has shown successful results in the treatment of human or animal models of diseases such as rheumatoid arthritis, Crohn's disease, and multiple sclerosis (Feldman et al, 1998, Transplant. Proc. 30: 4126-4127; Arend et al, 1998, C. Annu. Rev. Immunol. 16: 27-55; Bresnihan, B., 1999, Ann. Rheum. Dis. 58 Suppl 1: 196-198; Badovinac et al, 1998, J. Neuroimmunol. 85: 87-95; Wiemann et al, 1998, Exp. Neurol. 149: 455-463). Based on the concept that T lymphocytes play a pivotal role in the pathogenesis of chronic inflammatory diseases, it was demonstrated that direct cell-cell contact with stimulated T lymphocytes is a major stimulus triggering monocytes to produce large amounts of TNF-α and IL-1β (Burger D. and Dayer J. M., T Cells in Arthritis 111-128 (1998)).

Various stimuli are able to induce monocyte-activating capacity in T cells, including polyclonal mitogens such as a combination of phytohemagglutinin (PHA) and phorbol myristate acetate (PMA) (Vey et al, 1992, J. Immunol. 149: 2040-2046; Isler et al, 1993, Eur. Cytokine Netw. 4: 15-23; Lacraz et al, 1994, J. Biol. Chem. 269: 22027-22033; Li et al, 1995, Immunology 84: 571-576), cross-linking of CD3 by immobilized anti-CD3 mAb with or without cross-linking of the co-stimulatory molecule CD28-2-(Miltenburg et al, 1995, J. Immunol. 154: 2655-2667; Chizzolini et al, 1997, Eur. J. Immunol. 27: 171-177) and antigen-recognition on antigen-specific T cell clones (Chizzolini et al, 1997, Eur. J. Immunol. 27: 171-177).

The inventors believe that membrane-associated ligands on stimulated T cells trigger monocyte-macrophage signaling by binding to counter-ligands on monocytes. The identity of these ligands and counter-ligands, however, has been elusive. In the human system, part of the signaling might be attributed to β2-integrins, CD69, CD23, CD40-CD40L and lymphocyte activation gene-3 (LAG-3) (Vey et al, 1992, J. Immunol. 149: 2040-2046; Isler et al, 1993, Eur. Cytokine Netw. 4: 15-23; Lacraz et al, 1994, J. Biol. Chem. 269: 22027-22033; Hermann et al, 1999, J. Cell Biol. 144: 767-775; Stout et al, 1996, J. Immunol. 156: 8-11; Suttles et al, 1999, J. Biol. Chem. 274: 5835-5842; Avice et al, 1999, J. Immunol. 162: 2748-2753; Armant et al, 1995, J. Immunol. 155: 4868-4875; Rezzonico et al, (2000 in press), Blood). Membrane-associated TNF-α and IL-1β do not play a crucial part in this cellular interaction, contrasting with their significant role in stimulatory processes induced by stimulated T cells in human fibroblasts/synoviocytes or microvascular endothelial cells (Burger et al, 1998, Arthritis Rheum. 41: 1748-1759; Lou et al, 1996, Eur. J. Immunol. 26: 3107-3113; Burger et al, 1998, T Cells in Arthritis 111-128).

When assessing the inhibitory activity of human serum fractions on TNF-β and IL-1β production induced by T cell-signaling of monocytes or monocytic cells (THP-1 cells), the inventors determined that apo-A-1 was a serum inhibitory factor. This finding facilitates development of new compositions and methods for the treatment of diseases and conditions involving T cell-signaling of monocytes or monocytic cells.

According to certain embodiments, the invention provides polypeptides and nucleic acid molecules encoding the same to regulate T-cell-mediated activation of monocytes. According to other embodiments, the invention provides methods for the treatment and diagnosis of diseases and conditions that involve T-cell-mediated activation of monocytes.

### SUMMARY OF THE INVENTION

The invention provides for compositions of matter, processes and methods of useconcerning apo-A-I (SEQ ID NO:2) and fragments and derivatives thereof and their use in regulating T-cell-mediated activation of monocytes. According to certain embodiments, the invention concerns an isolated nucleic acid molecule consisting essentially of a nucleotide sequence selected from:

(a) the nucleotide sequence as set forth in residues 73 to 601 in SEQ ID NO: 1;

(b) a nucleotide sequence encoding the polypeptide as set forth in residues 25 to 194 in SEQ ID NO:2;

(c) the nucleotide sequence as set forth in residues 73 to 451 in SEQ ID NO: 1;

(d) a nucleotide sequence encoding the polypeptide as set forth in residues 25 to 144 in SEQ ID NO:2;

(e) the nucleotide sequence as set forth in residues 485 to 820 in SEQ ID NO: 1;

(f) a nucleotide sequence encoding the polypeptide as set forth in residues 25 to 113 in SEQ ID NO:2;

(g) a nucleotide sequence encoding the polypeptide as set forth in residues 73 to 113 in SEQ ID NO:2;

(h) a nucleotide sequence encoding the polypeptide as set forth in residues 156 to 267 in SEQ ID NO:2;

(i) a nucleotide sequence which hybridizes under moderately or highly stringent conditions to the complement of at least one of (a) to (f), wherein the encoded polypeptide has an activity of the polypeptide as set forth in SEQ ID NO: 2; and

(j) a nucleotide sequence complementary to at least one of (a)-(h).

In certain other embodiments, the invention relates to an isolated polypeptide consisting essentially of an amino acid sequence selected from:

(a) an amino acid sequence as set forth in residues 25 to 194 of SEQ ID NO:2;

(b) an amino acid sequence as set forth in residues 25 to 144 of SEQ ID NO:2;

(c) an amino acid sequence as set forth in residues 156 to 267 of SEQ ID NO:2;

(d) an amino acid sequence as set forth in residues 25 to 113 of SEQ ID NO:2;

(e) an amino acid sequence as set forth in residues 75 to 113 of SEQ ID NO:2;

(f) an amino acid sequence for an ortholog of SEQ ID NO:2, wherein the encoded polypeptide has an activity of the polypeptide as set forth in SEQ ID NO:2;

(g) an amino acid sequence that is at least about 70, 80, 85, 90, 95, 96, 97, 98, or 99 percent identical to the amino acid sequence of at least one of (a), (b), or (c), wherein the polypeptide has an activity of the polypeptide as set forth in SEQ ID NO:2;

(h) a fragment of the amino acid sequence set forth in at least one of (a), (b), (c), (d), or (e) comprising at least about 25 amino acid residues, wherein the polypeptide has an activity of a polypeptide as set forth in SEQ ID NO:2;

(i) an amino acid sequence for an allelic variant or splice variant of at least one of (a)-(f) wherein the polypeptide has an activity of a polypeptide as set forth in SEQ ID NO:2.

The invention concerns in certain embodiments the polypeptide fragments described in residues 25-113, 73-113, 25-194, 25-144, or 156-267 of SEQ ID NO:2 and related polypeptides, which are apo-A-1 fragment T-cell activation inhibitors (“AFTIs”). In certain embodiments, the AFTIs of the invention include, but are not limited to, an N-terminal fragment of apo-A-1, beginning at residue number 25 of SEQ ID NO:2, and comprising a fragment of 13 kilodalton (kDa) or 18 kDa of the polypeptide shown in SEQ ID NO:2. In certain other embodiments, the AFTIs of the invention include, but are not limited to, a C-terminal fragment of apo-A-1, ending at the last amino acid residue of SEQ ID NO:2, and comprising a fragment of 13 kDa of the polypeptide shown in SEQ ID NO:2.

In certain embodiments, the invention further provides for an isolated polypeptide comprising the amino acid sequence selected from:

(a) the amino acid sequence as set forth in SEQ ID NO:2, or residues 25-113, 73-113, 25-194, 25-144, or 156-267 as set forth in SEQ ID NO:2, with at least one conservative amino acid substitution, wherein the polypeptide has an activity of the polypeptide as set forth in SEQ ID NO:2;

(b) the amino acid sequence as set forth in SEQ ID NO:2, or residues 25-113, 73-113, 25-194, 25-144, or 156-267 as set forth in SEQ ID NO:2, with at least one amino acid insertion, wherein the polypeptide has an activity of the polypeptide as set forth in SEQ ID NO:2;

(c) the amino acid sequence as set forth in SEQ ID NO:2, or residues 25-113, 73-113, 25-194, 25-144, or 156-267 as set forth in SEQ ID NO:2, with at least one amino acid deletion, wherein the polypeptide has an activity of the polypeptide as set forth in SEQ ID NO:2;

(d) the amino acid sequence as set forth in SEQ ID NO:2, or residues 25-113, 73-113, 25-194, 25-144, or 156-267 as set forth in SEQ ID NO:2, which has a C- and/or N-terminal truncation, wherein the polypeptide has an activity of the polypeptide as set forth in SEQ ID NO:2; and

(e) the amino acid sequence as set forth in SEQ ID NO:2, or residues 25-113, 73-113, 25-194, 25-144, or 156-267 as set forth in SEQ ID NO:2, with at least one modification selected from the group consisting of amino acid substitutions, amino acid insertions, amino acid deletions, C-terminal truncation, and N-terminal truncation, wherein the polypeptide has an activity of the polypeptide as set forth in SEQ ID NO:2.

In certain embodiments, the invention provides fusion polypeptides comprising a polypeptide described herein (e.g., the amino acid sequences of (a)-(e) above) and a heterologous polypeptide. In certain preferred embodiments, the heterologous polypeptide is an Fc domain.

In certain embodiments, the invention provides for an isolated nucleic acid molecule comprising a nucleotide sequence selected from the group consisting of:

(a) a nucleotide sequence encoding a polypeptide as set forth in SEQ ID NO:2, or residues 25-113, 73-113, 25-194, 25-144, or 156-267 as set forth in SEQ ID NO:2;

(b) a nucleotide sequence encoding a polypeptide as set forth in SEQ ID NO:2, or residues 25-113, 73-113, 25-194, 25-144, or 156-267 as set forth in SEQ ID NO:2, with at least one conservative amino acid substitution, wherein the polypeptide has an activity of the polypeptide as set forth in SEQ ID NO:2;

(c) a nucleotide sequence encoding a polypeptide as set forth in SEQ ID NO:2, or residues 25-113, 73-113, 25-194, 25-144, or 156-267 as set forth in SEQ ID NO:2, with at least one amino acid insertion, wherein the polypeptide has an activity of the polypeptide as set forth in SEQ ID NO:2;

(d) a nucleotide sequence encoding a polypeptide as set forth in SEQ ID NO:2, or residues 25-113, 73-113, 25-194, 25-144, or 156-267 as set forth in SEQ ID NO:2, with at least one amino acid deletion, wherein the polypeptide has an activity of the polypeptide as set forth in SEQ ID NO:2;

(e) a nucleotide sequence encoding a polypeptide as set forth in SEQ ID NO:2, or residues 25-113, 73-113, 25-194, 25-144, or 156-267 as set forth in SEQ ID NO:2, which has a C- and/or N- terminal truncation, wherein the polypeptide has an activity of the polypeptide as set forth in SEQ ID NO:2;

(f) a nucleotide sequence encoding a polypeptide as set forth in SEQ ID NO:2, or residues 25-113, 73-113, 25-194, 25-144, or 156-267 as set forth in SEQ ID NO:2, with at least one modification selected from the group consisting of amino acid substitutions, amino acid insertions, amino acid deletions, C-terminal truncation, and N-terminal truncation, wherein the polypeptide has an activity of the polypeptide as set forth in SEQ ID NO:2;

(g) a nucleotide sequence of (a)-(f) comprising a fragment of at least about 16 nucleotides;

(h) a nucleotide sequence that hybridizes under moderately or highly stringent conditions to the complement of any of (a)-(g), wherein the polypeptide encoded has an activity of the polypeptide as set forth in SEQ ID NO:2; and

(i) a nucleotide sequence complementary to any of (a)-(f).

In certain embodiments, the present invention provides for an expression vector comprising the isolated nucleic acid molecules as set forth herein, recombinant host cells comprising recombinant nucleic acid molecules as set forth herein, and a method of producing an AFTI polypeptide comprising culturing the host cells and optionally isolating the polypeptide so produced.

A transgenic non-human animal comprising an engineered nucleic acid molecule encoding an AFTI polypeptide is provided by certain embodiments of the invention. In certain embodiments, the AFTI nucleic acid molecules are introduced into the animal in a manner that allows expression, preferably in increased levels of the AFTI polypeptide, which may include increased levels of the AFTI polypeptide in the animal (e.g., the bloodstream). According to certain embodiments, the transgenic non-human animal may be a mammal, for example, a rodent, a rat, a mouse, a cow, a sheep, a goat, a cow, a dog, a cat, etc.

Also provided in certain embodiments are derivatives of the AFTI polypeptides of the present invention.

Additionally provided in certain embodiments are selective binding agents such as antibodies and peptides capable of specifically binding the AFTI polypeptides of the invention. Such antibodies and peptides may be agonistic or antagonistic.

Pharmaceutical compositions comprising a polynucleotide, a polypeptide, and/or a selective binding agent of the present invention and one or more pharmaceutically acceptable formulation agents are also encompassed by certain embodiments of the invention. In certain embodiments, the pharmaceutical compositions are used to provide therapeutically or diagnostically effective amounts of the nucleotides or polypeptides of the present invention. The invention, according to certain embodiments, is directed to methods of using the polypeptides, nucleic acid molecules, and selective binding agents.

According to certain embodiments, the AFTI polypeptides and nucleic acid molecules of the present invention may be used to treat, prevent, ameliorate, and/or detect diseases and disorders, including those recited herein.

According to certain embodiments, the present invention provides a method of assaying test molecules to identify a test molecule that binds to an AFTI polypeptide. According to certain embodiments, the method comprises contacting an AFTI polypeptide with a test molecule and determining the extent of binding of the test molecule to the polypeptide. According to certain embodiments, the method comprises determining whether such test molecules are agonists or antagonists of an AFTI polypeptide. According to certain embodiments, the present invention further provides a method of testing the impact of molecules on the expression of AFTI polypeptide or on the activity of AFTI polypeptide.

Methods of regulating expression and modulating (i.e., increasing or decreasing) levels of an AFTI polypeptide are also encompassed by certain embodiments of the invention. Certain methods comprise administering to an animal a nucleic acid molecule encoding an AFTI polypeptide. In another method, a nucleic acid molecule comprising elements that regulate or modulate the expression of an AFTI polypeptide may be administered. Examples of these methods include, but are not limited to, gene therapy, cell therapy, and anti-sense therapy as further described herein.

In certain embodiments of the present invention, the AFTI polypeptides may be used for identifying AFTI receptors. Various forms of “expression cloning” have been extensively used for cloning receptors for protein ligands. See for example, H. Simonsen and H. F. Lodish, Trends in Pharmacological Sciences, vol. 15, 437-441 (1994), and Tartaglia etai, Cell, 83: 1263-1271 (1995). In certain embodiments, the isolation of the AFTI receptor(s) is useful for identifying or developing novel agonists and antagonists of the AFTI polypeptide-signaling pathway. Such agonists and antagonists include, but are not limited to, soluble AFTI receptor(s), anti-AFTI receptor selective binding agents (such as antibodies and derivatives thereof), small molecules, and antisense oligonucleotides, any of which can be used for treating one or more of the diseases or disorders, including those recited herein.

### BRIEF DESCRIPTION OF THE FIGURES

FIG. 1: (A) Human apolipoprotein A-1 amino acid sequence (SEQ ID NO:2) and polynucleotide sequence (SEQ ID NO: 1). Apo A-1 polypeptide has helical lipid binding domains (amino acid residues 44-65 and 220-241), a domain involved in lipoprotein-mediated cholesterol efflux from monocytes (amino acid residues 74-111), a receptor binding domain (amino acid residues 149-219), a major antigenic epitope domain (amino acid residues 99-120), a hinged domain (amino acid residues 99-143), a phylogenetically conserved domain (amino acid residues 66-120), and a domain involved in lectin-cholesterol acyltransferase activity (amino acid residues 90-111). The apo-A-1 polypeptide has eight amphipathic helices (amino acid residues 44-65, 66-98, 99-120, 121-142, 143-164, 165-208, 209-219, 220-241), an N-terminal peptide (amino acid residues 1-43), and a C-terminal peptide (amino acid residues 242-243). AFTI amino acid sequences include, but are not limited to, fragments of SEQ ID NO:2, for example, (B) a 18 kDa N-terminal fragment (amino acid residues 25-194 (SEQ ID NO:3), nucleotides 92-601 (SEQ ID NO:4)), (C) a 13 kDa N-terminal fragment (amino acid residues 25-144, nucleotides 92-451), and (D) a 13 kDa C-terminal fragment (amino acid residues 156-267, nucleotides 485-820).

FIG. 2: Human serum inhibits TNF-α and IL-1β production in PHA-stimulated PBMC. PBMC (4×10⁵ cells/200 μl/well) were stimulated with 1 μg/ml PHA in medium containing either FCS or HS. (A) TNF-α and Iβ were measured in supernatant after 48 h incubation; (B) proliferation (³H-thymidine incorporation) was measured after 72 h.

FIG. 3: Inhibition of T cell signaling of monocytes and THP-1 cells by HS. (A) THP-1 cells were activated for 48 hours by fixed, stimulated T lymphocytes at a cellular ratio of 8 T lymphocytes/THP-1 cell in the presence of increasing doses of HS (HS, closed symbols) or FCS (open symbols). (B) THP-1 cells were activated for 48 hours by either fixed, stimulated HUT-78 cells at a cellular ratio of 8 HUT-78 cells/THP-1 cell (closed symbols) or 10 μg/ml lipopolysaccharide (LPS) and 5 g/ml PMA (open symbols) in the presence of increasing doses of HS. THP-1 cells (C and D) and monocytes (E and F) were activated for 48 hours by increasing doses of membranes isolated from stimulated HUT-78 cells in the presence or absence of 10% HS. TNF-α (C and E) and IL-1β (D and F) were measured in culture supernatants. Results represent mean ±SD, n=3 except in (B) where n=7. In A and B, 100% represent the production of IL-1β after 48 hours culture in the absence of inhibitor.

FIG. 4: Superdex 200 elution profile and SDS-PAGE analysis of serial chromatography fractionation of HS. (A) Inhibitory fractions eluted from Phenyl Sepharose HP were pooled, concentrated and subjected to gel filtration on Superdex 200 equilibrated in PBS. The column was calibrated with the molecular weight marker kit for gel filtration chromatography (Sigma). Fractions were tested for their protein contents (OD_{280 mm}, dashed line) and inhibitory activity (closed circles). (B) Inhibitory fractions were pooled after each step and 10 μg protein aliquots were loaded per lane on a 10% polyacrylamide gel; (a) HS; (b) breakthrough of Blue-Sepharose®; (c) pool of inhibitory fractions from Q Sepharose(g; (d) pool of inhibitory fractions from Phenyl Sepharose® HP; (e) pool of inhibitory fractions from Superdex® 200; the gel was stained with Coomassie blue.

FIG. 5: Presence of the inhibitory activity in protein fractions of HDL. HS was fractionated by high density centrifugation and the inhibitory activity of HDL and serum protein fractions was analyzed. Isolated HDL were further subjected to either delipidation (delipidated HDL) or proteolytic digestion with proteinase K (Proteinase K treated HDL). The inhibitory activity of fractions was compared to HS (whole serum). The final protein concentration for whole serum and serum proteins was 7 mg/ml (black columns), 3.5 mg/ml (gray columns), and 0.7 mg/ml (white columns). The final protein concentration for HDL and delipidated HDL was 0.2 mg/ml (black columns), 0.1 mg/ml (gray columns), and 0.02 mg/ml (white columns). The amount of proteinase K-treated HDL was estimated according to the protein concentration before proteolysis and was similar to untreated HDL. Results represent the percentage of IL-1β or TNF-α production in the absence of inhibitor (mean ±SD, n=3).

FIG. 6: Analysis of HDL binding to cells. (A) Inhibition of T cell-signalling by binding of HDL to membranes of stimulated HUT-78 cells; either membranes of stimulated HUT-78 cells (white columns), THP-1 cells (hatched columns), or both (black columns) were preincubated in the absence (−) or presence of FCS (10%), HS (10%) or HDL (0.32 mg/ml protein) for 45 minutes on ice; after washing, treated (hatched and black columns) and untreated (white columns) THP-1 cells were cultured in the presence of treated (white and black columns) or untreated (hatched columns) membranes of stimulated HUT-78 ceils; TNF-α and IL-1β production was measured in 48 hours-culture supernatants. Results are expressed as percentage considering the production measured in the absence of inhibitor as 100%, mean ±SD, n=6. (B-F) Binding of unconjugated FITC and FITC-HDL (0.1 mg/ml) was assessed by flow cytometry on THP-1 cells (B), isolated human monocytes (C), unstimulated HUT-78 cells (D) and stimulated HUT-78 cells (E). FITC was used as a negative control. (F) Binding of FITC-HDL (10 μg/ml) to stimulated HUT-78 cells in the presence or absence of purified anti-apo-A-I antibodies (100 μg/ml) (ATCC, Manassas, Va.; catalogue number HB-9570).

FIG. 7: Apo-A-I inhibits the production of TNF-α and IL-β in THP-1 cells activated by membranes of stimulated HUT-78 cells. (A) THP-1 cells were activated by membranes of stimulated HUT-78 cells in the presence of increasing concentrations of apo-A-1 purchased from Sigma (St. Louis, Mo.); After 48 h, TNF-α and IL-1β were measured in culture supernatants. Results represent mean ±SD, n=3. (B and C) THP-1 cells were activated by membranes of stimulated HUT-78 cells in the presence of increasing concentration of proteins electroeluted from preparative SDS-PAGE of delipidated HDL: (B) (Mᵣ=28,000, and (C) (Mᵣ=18,000. (D) THP-1 cells were activated by membranes of stimulated HUT-78 cells in the presence of increasing concentrations of apo-A-1 (Mᵣ=28,000) isolated by gel filtration on Superdex S75. After 48 hours, TNF-α and IL-1β were measured in culture supernatants. Results represent mean ±SD=3, 100% being the amount of cytokine produced in the absence of inhibitor. (E) Isolated fractions that were tested for inhibitory activity were analyzed for their apo-A-1 content by western blotting; (a) 28,000 electroeluted band, (b) 18,000 electroeluted band, and (c) 28,000 protein recovered from Superdex® S75 gel filtration.

FIG. 8: Apo-A-1 decreases the steady-state levels of TNF-α and IL-β mRNA. (A and B) Autoradiogram of RNase protection assay. (A) THP-1 cells (5×10⁶ cell/ml) untreated (a) or activated by membranes of stimulated HUT cells (200 μg protein/ml) during 3 h (b-e) in the presence or absence of apo-A-1 (200 μg/ml) (c-e) which was added at different time points of THP-1 activation: c (0 h); d (1 h); and e (2 h). (B) Monocytes (10×10⁶ cell/ml) untreated (a) or activated by membranes of stimulated T lymphocytes (40 μg protein/ml) during 1 hour (b-e) in the presence or absence of Apo A-1 (200 μg/ml) (c-e) which was added at different time points of THP-1 activation: c (0 minutes); d (15 minutes); and e (30 minutes). (C and D) Densitometric analysis of autoradiography A and B, respectively, normalized with the densitometry of GAPDH mRNA=1, and expressed as percentage considering the mRNA level of activated THP-1 cells (B) or monocytes (C) in the absence of inhibitor as 100%. Results are representative of 3 different experiments.

FIG. 9: Apo A-1 inhibits TNFα and IL-1β in PBMC stimulated bu either PHA or Tetanus Toxoid (TT). PBMC 4×10⁵ cells/200 μl/well were stimulated by 1 μg/ml PHA (A and B) or by 10 μg/ml TT (C and D) in the presence of the indicated doses of apo A-1 and HDL.

FIG. 10: Recombinant human apo A-1 Milano displays the inhibitory activity. THP-1 cells were stimulated with membranes of stimulated HUT-78 cells in the presence of serial dilutions of human serum (HS), apolipoprotein A-1 from Sigma (starting concentration=2 mg/ml), and recombinant apolipoprotein A-1 Milano (starting concentration=2 mg/ml). Both apolipoproteins (purified and recombinant) displayed inhibitory activity.

FIG. 11: Inhibition of contact-mediated activation of THP-1 cells by apo A-11 and a fragment of apo A-1. THP-1 cells were activated by membranes of stimulated HUT-78 cells (HUTs) in the presence of the indicated inhibitor. Both IL-1β and TNF-α production were inhibited by apo A-1, apo A-11 and a fragment containing domains II and III of apo A-1.

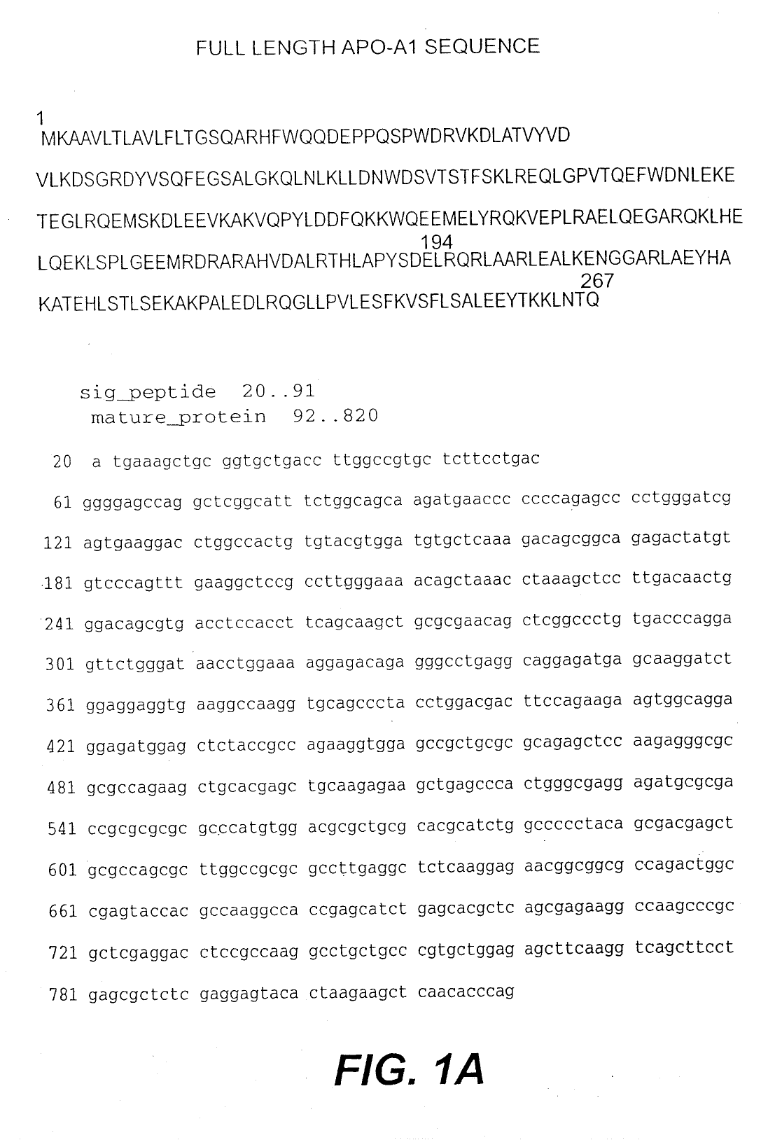

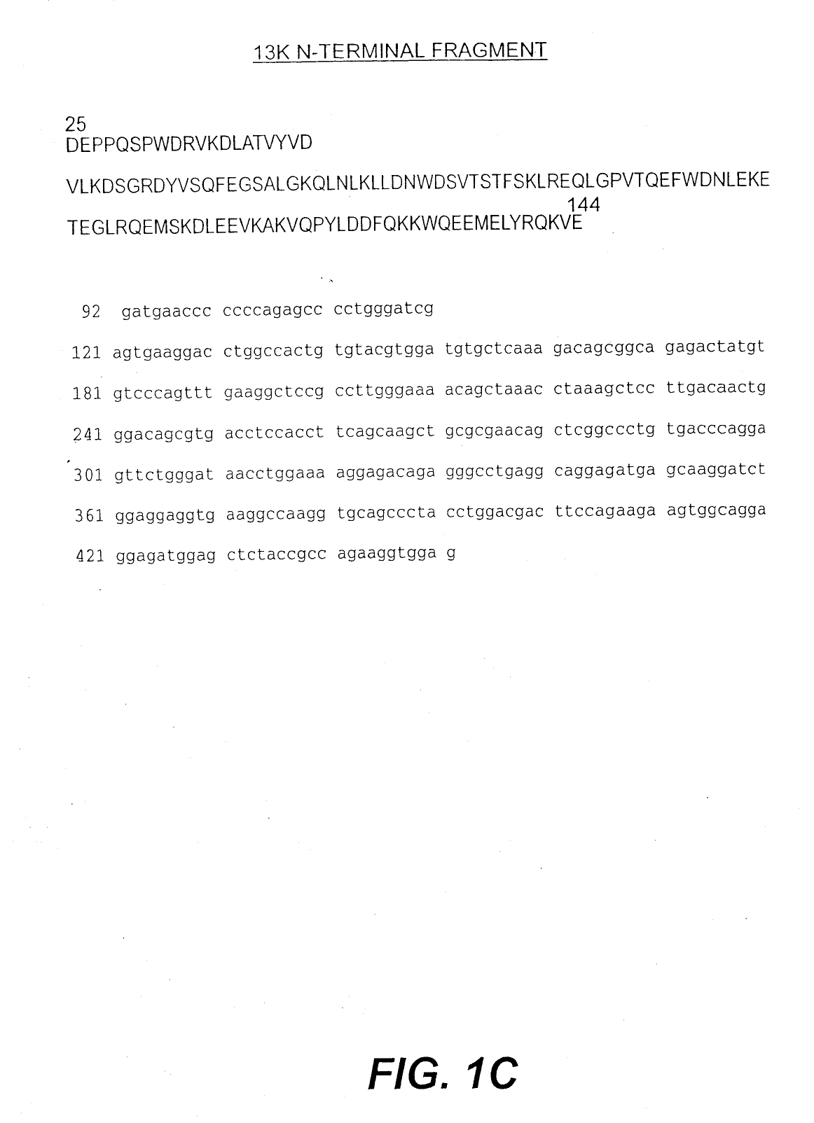

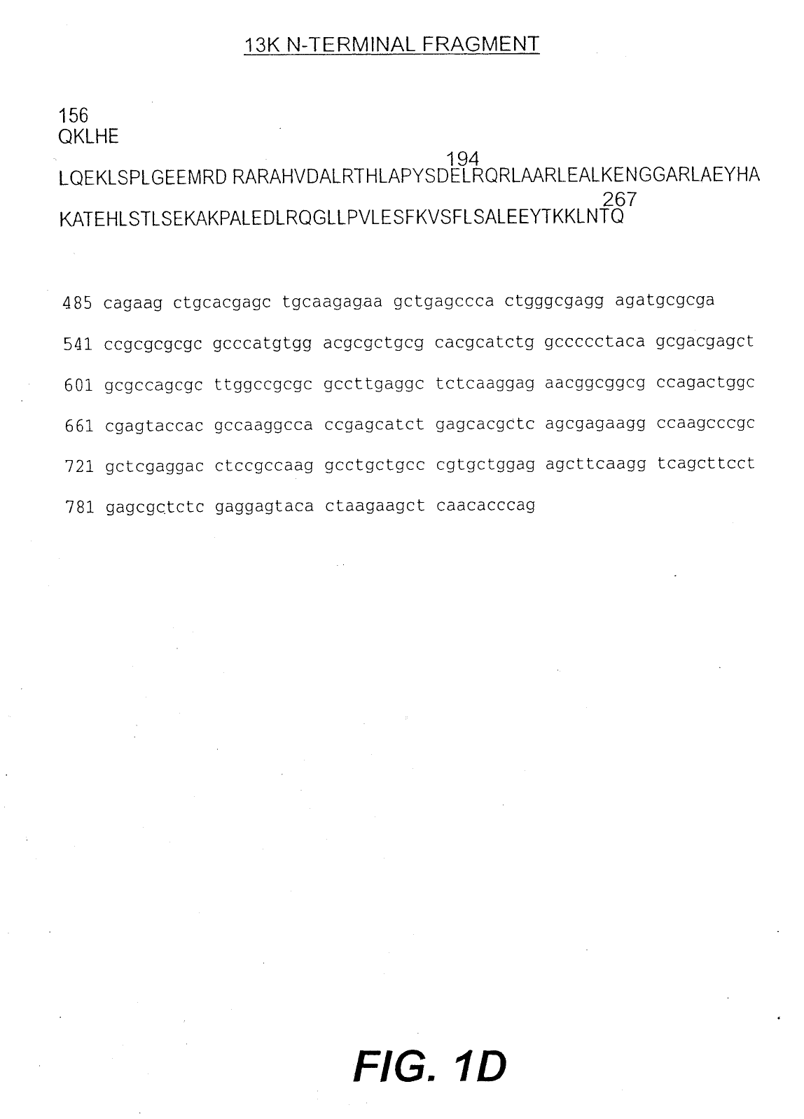

### DETAILED DESCRIPTION OF THE INVENTION

The section headings used herein are for organizational purposes only and are not to be construed as limiting the subject matter described. All references cited in this application are expressly incorporated herein by reference in their entirety for any purpose, except where such incorporation would define a term differently from the meanings provided below.

Definitions

The terms “AFTI gene” or “AFTI nucleic acid molecule” or “AFTI polynucleotide” refer to a nucleic acid molecule having a nucleotide sequence as set forth in SEQ ID NO:1 or any segment thereof, a nucleotide sequence encoding the polypeptide as set forth in SEQ ID NO:2, or any fragment thereof.

The term “AFTI polypeptide” refers to a polypeptide having the amino acid sequence of SEQ ID NO:2, or any fragment thereof, and related polypeptides. Related polypeptides include: AFTI polypeptide allelic variants, AFTI polypeptide orthologs, AFTI polypeptide splice variants, AFTI polypeptide variants and AFTI polypeptide derivatives. AFTI polypeptides may be mature polypeptides, as defined herein, and may or may not have an amino terminal methionine residue, depending on the method by which they are prepared.

The term “AFTI polypeptide allelic variant” refers to one of several or many possible naturally occurring alternate forms of a gene occupying a given locus on a chromosome of an organism or a population of organisms.

The term “AFTI polypeptide derivatives” refers to a polypeptide fragment of the

polypeptide shown in SEQ ID NO:2, AFTI polypeptide allelic variants, AFTI polypeptide orthologs, AFTI polypeptide splice variants, or AFTI polypeptide variants, as defined herein, that have been chemically modified.

The term “AFTI polypeptide fragment” refers to a polypeptide that has a truncation at the amino terminus (with or without a leader sequence) and/or a truncation at the carboxy terminus of an AFTI polypeptide described herein, AFTI polypeptide allelic variants, AFTI polypeptide orthologs, AFTI polypeptide splice variants and/or an AFTI polypeptide variant having one or more amino acid additions or substitutions or internal deletions (wherein the resulting polypeptide is at least 6 amino acids in length) as compared to an AFTI polypeptide amino acid sequence specifically described herein. An AFTI polypeptide fragment may result, for example, from alternative RNA splicing or from in vivo protease activity. In preferred embodiments, truncations comprise about 10 amino acids, or about 20 amino acids, or about 50 amino acids, or about 75 amino acids, or about 100 amino acids, or more than about 100 amino acids. The polypeptide fragments so produced will comprise about 25 contiguous amino acids, or about 50 amino acids, or about 75 amino acids, or about 100 amino acids, or about 150 amino acids, or about 200 amino acids. In certain embodiments, an AFTI polypeptide fragment of the invention is from 6 amino acids in length up to a polypeptide described herein, or any number of amino acids between those sizes. Such AFTI polypeptide fragments may optionally comprise an amino terminal methionine residue. It will be appreciated that such fragments can be used, for example, to generate antibodies to AFTI polypeptides.

The term “AFTI fusion polypeptide” refers to a fusion of one or more amino acids (such as a heterologous peptide or polypeptide) at the amino or carboxy terminus of an AFTI polypeptide or an AFTI polypeptide fragment. Thus, this term includes fusion proteins having the sequence of any of the polypeptides specifically described herein, AFTI polypeptide allelic variants, AFTI polypeptide orthologs, AFTI polypeptide splice variants, or AFTI polypeptide variants having one or more amino acid deletions, substitutions or internal additions as compared to an AFTI polypeptide amino acid sequence specifically described herein.

The term “AFTI polypeptide ortholog” refers to a polypeptide from another species that corresponds to an AFTI polypeptide amino acid sequence specifically described herein. For example, mouse and human AFTI polypeptides are considered orthologs of each other.

The term “AFTI polypeptide splice variant” refers to an AFTI polypeptide encoded by a nucleic acid molecule, usually RNA, that is generated by alternative processing (e.g., alternative splicing) of intron and/or exon sequences in an RNA transcript corresponding to an AFTI polypeptide specifically described herein.

The term “AFTI polypeptide variants” refers to AFTI polypeptides comprising amino acid sequences having one or more amino acid sequence substitutions, deletions (such as, for example, internal deletions and/or AFTI polypeptide fragments), and/or additions (such as, for example, internal additions and/or AFTI fusion polypeptides) as compared to an AFTI polypeptide amino acid sequence specifically described herein (with or without a leader sequence). Variants may be naturally occurring (e.g., AFTI polypeptide allelic variants, AFTI polypeptide orthologs and AFTI polypeptide splice variants) or artificially constructed. Such AFTI polypeptide variants may be prepared from the corresponding nucleic acid molecules having a DNA sequence that varies accordingly from the DNA sequence as set forth in SEQ ID NO:1 that encodes an AFTI of interest. In preferred embodiments, the variants have from 1 to 3, or from 1 to 5, or from 1 to 10, or from 1 to 15, or from 1 to 20, or from 1 to 25, or from 1 to 50, or from 1 to 75, or from 1 to 100, or more than 100 amino acid substitutions, insertions, additions and/or deletions, wherein the substitutions may be conservative, or non-conservative, or any combination thereof.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “biologically active AFTI polypeptides” refers to AFTI polypeptides having at least one activity characteristic of a polypeptide comprising the amino acid sequence of SEQ ID NO:1.

The terms “effective amount” and “therapeutically effective amount” each refer to the amount of a AFTI polypeptide or AFTI nucleic acid molecule used to support an observable level of one or more biological activities of the AFTI polypeptides as set forth herein.

The term “expression vector” refers to a vector that is suitable for use in a host cell and contains one or more nucleic acid sequences that direct and/or control the expression of heterologous nucleic acid sequences. Expression includes, but is not limited to, one or more processes such as transcription, translation, and RNA splicing (if introns are present).

The term “host cell” is used to refer to a cell that has been transfected or transformed, or is capable of being transfected or transformed with a nucleic acid sequence of interest and that is capable of expressing the nucleic acid of interest (or a segment of the nucleic acid of interest). The term includes the progeny of the parent cell, whether or not the progeny is identical in morphology or in genetic make-up to the original parent, so long as the nucleic acid of interest is present.

The term “identity” as known in the art, refers to a relationship between the sequences of two or more polypeptide molecules or two or more nucleic acid molecules, as determined by comparing the sequences. In the art, “identity” also means the degree of sequence relatedness between nucleic acid molecules or between polypeptides, as the case may be, as determined by the number of matches between strings of two or more nucleotide residues or two or more amino acid residues. “Identity” measures the percent of identical matches between the smaller of two or more sequences with gap alignments (if any) addressed by a particular mathematical model or computer program (i.e., “algorithms”).

The term “similarity” is a related concept, but in contrast to “identity”, refers to a sequence relationship that includes both identical matches and conservative substitution matches. If two polypeptide sequences have, for example, 10/20 identical amino acids, and the remainder are all non-conservative substitutions, then the percent identity and similarity would both be 50%. If, in the same example, there are 5 more positions where there are conservative substitutions, then the percent identity remains 50%, but the percent similarity would be 75% (15/20). Therefore, in cases where there are conservative substitutions, the degree of similarity between two polypeptides will be higher than the percent identity between those two polypeptides.

The term “isolated nucleic acid molecule” refers to a nucleic acid molecule of the invention that (1) has been separated from at least about 50 percent of proteins, lipids, carbohydrates or other materials (i.e., contaminants) with which it is naturally associated, (2) is not covalently linked to all or a portion of a polynucleotide to which the “isolated nucleic acid molecule” is covalently linked in nature, (3) is operably linked covalently to a polynucleotide that it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use.

The term “isolated polypeptide” refers to a polypeptide of the present invention that (1) has been separated from at least about 50 percent of polynucleotides, lipids, carbohydrates or other materials (i.e., contaminants) with which it is naturally associated, (2) is not covalently linked to all or a portion of a polypeptide to which the “isolated polypeptide” is linked in nature, (3) is operably linked covalently to a polypeptide to which it is not covalently linked in nature, or (4) does not occur in nature. Preferably, the isolated polypeptide is substantially free from any other contaminating polypeptides or other contaminants that are found in its natural environment which would interfere with its therapeutic, diagnostic, prophylactic or research use.

The term “nucleic acid sequence” or “nucleic acid molecule” refers to a polymer of at least two nucleotides. Such nucleotides include all naturally occurring nucleotides and all synthetic nucleotides. Such nucleotides may be formed, for example, from any of the known base analogs of DNA and RNA including, but not limited to, 4-acetylcytosine, 8-hydroxy-N6-methyladenosine, aziridinyl-cytosine, pseudoisocytosine, 5-(carboxyhydroxylmethyl) uracil, 5- fluorouracil, 5-bromouracil, 5-carboxymethylaminomethyl-2-thiouracil, 5-carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5- methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “naturally occurring” or “native” when used in connection with biological materials such as nucleic acid molecules, polypeptides, host cells, and the like, refers to materials that are found in nature and are not manipulated (i.e., designed or engineered) by man. Similarly, “non-naturally occurring” or “non-native” as used herein refers to a material that is not found in nature or that has been structurally modified or synthesized by man.

The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “pharmaceutically acceptable carrier” or “physiologically acceptable carrier” as used herein refers to one or more formulation materials suitable for accomplishing or enhancing the delivery of the AFTI polypeptide, AFTI nucleic acid molecule or AFTI selective binding agent as a pharmaceutical composition.

The term “selective binding agent” refers to a molecule or molecules having specificity for an AFTI polypeptide. As used herein, the terms, “specific” and “specificity” refer to the ability of the selective binding agents to bind to human AFTI polypeptides and not to bind to human non-AFTI polypeptides. It will be appreciated, however, that the selective binding agents may also bind orthologs of the polypeptide as set forth in SEQ ID NO:2, that is, interspecies versions thereof, such as mouse and rat polypeptides.

The term “transduction” is used to refer to the transfer of genes from one bacterium to another, usually by a phage. “Transduction” also refers to the acquisition and transfer of eukaryotic cellular sequences by retroviruses.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, for example, Graham et al., Virology, 52: 456 (1973); Sambrook et al., Molecular Cloning, A Laboratory Manual, Cold Spring Harbor Laboratories (New York, 1989); Davis et al, Basic Methods in Molecular Biology, Elsevier, 1986; and Chu et al., Gene, 13: 197 (1981). Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection ortransduction, the transforming DNA may recombine with that of the cell by physically integrating into a chromosome of the cell, may be maintained transiently as an episomal element without being replicated, or may replicate independently as a plasmid. A cell is considered to have been stably transformed when the DNA is replicated with the division of the cell.

The term “vector” is used to refer to any molecule (e.g., nucleic acid, plasmid, or virus) used to transfer coding information to a host cell.

Relatedness of Nucleic Acid Molecules and/or Polypeptides

It is understood that related nucleic acid molecules include allelic or splice variants of the nucleic acid molecule of SEQ ID NO:1 or fragments thereof, and include sequences that are complementary to any of the above nucleotide sequences. Related nucleic acid molecules also include a nucleotide sequence encoding a polypeptide comprising, or consisting essentially of, or consisting of a substitution, modification, addition and/or a deletion of one or more amino acid residues compared to a polypeptide described herein, e.g., the polypeptide in SEQ ID NO:2.

Fragments include molecules that encode a polypeptide of at least about 25 amino acid residues, or about 50, or about 75, or about 100, or greater than about 100 amino acid residues of the polypeptide of SEQ ID NO:2.

In addition, related AFTI nucleic acid molecules include those molecules that comprise nucleotide sequences that hybridize under moderately stringent or highly stringent conditions as defined herein with the fully complementary sequence of the nucleic acid molecule of SEQ ID NO:1, or of a molecule encoding a polypeptide, which polypeptide comprises the amino acid sequence as shown in SEQ ID NO:2, or of a nucleic acid fragment as defined herein, or of a nucleic acid fragment encoding a polypeptide as defined herein. Hybridization probes may be prepared based on the AFTI sequences provided herein in order to screen cDNA, genomic or synthetic DNA libraries for related sequences. Regions of the AFTI DNA and/or the amino acid sequence of AFTI polypeptide that exhibit significant identity to known sequences are readily determined using sequence alignment algorithms as described herein and those regions may be used to design probes for screening.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual, 2^{nd} Ed., Cold Spring Harbor Laboratory, (Cold Spring Harbor, N.Y. 1989); Anderson et al., Nucleic Acid Hybridisation: a practical approach, Ch. 4, IRL Press Limited (Oxford, England).

More stringent conditions (such as higher temperature, lower ionic strength, higher formamide, or other denaturing agent) may also be used, however, the rate of hybridization will be affected. Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate (NaDodSO₄ or SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al. Nucleic Acid Hybridisation: a Practical Approach, Ch. 4, IRL Press Limited (Oxford, England).

Factors affecting the stability of a DNA duplex include base composition, length, and degree of base pair mismatch. Hybridization conditions can be adjusted by one skilled in the art in order to accommodate these variables and allow DNAs of different sequence relatedness to form hybrids. The melting temperature of a perfectly matched DNA duplex can be estimated by the following equation:

Tₘ(° C.)=81.5+16.6(log[Na+])+0.41 (%G+C)−600/N −0.72(% formamide) where N is the length of the duplex formed, [Na+] is the molar concentration of the polypeptidesodium ion in the hybridization or washing solution, % G+C is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex is able to form that has a greater degree of base pair mismatching than a DNA duplex able to form under “highly stringent conditions”. Examples of typical “moderately stringent conditions” are 0.015 M sodium chloride, 0.0015 M sodium citrate at 50-65° C. or 0.015 M sodium chloride, 0.0015 M sodium citrate, and 20% formamide at 37-50° C. By way of example, a “moderately stringent” condition of 50° C. in 0.015 M sodium ion will allow about a 21% mismatch.

It will be appreciated by those skilled in the art that there is no absolute distinction between “highly” and “moderately” stringent conditions. For example, at 0.015 M sodium ion (no formamide), the melting temperature of perfectly matched long DNA is about 71° C. With a wash at 65° C. (at the same ionic strength), this would allow for approximately a 6% mismatch. To capture more distantly related sequences, one skilled in the art can simply lower the temperature or raise the ionic strength.

A good estimate of the melting temperature in 1 M NaCl* for oligonucleotide probes up to about 20 nt is given by:

Tm=2° C. per A-T base pair+4° C. per G-C base pair

*The sodium ion concentration in 6× salt sodium citrate (SSC) is 1 M. See Suggs et al., Developmental Biology Using Purified Genes, p. 683, Brown and Fox (eds.)(1981).

High stringency washing conditions for oligonucleotides are usually at a temperature of 0-5° C. below the Tm of the oligonucleotide in 6× SSC, 0.1% SDS.

In another embodiment, related nucleic acid molecules comprise, or consist essentially of, or consist of a nucleotide sequence that is about 70 percent identical to the nucleotide sequence as shown in SEQ ID NO:1, or comprise, or consist essentially of, or consist of a nucleotide sequence encoding a polypeptide that is about 70 percent identical to the polypeptide as set forth in SEQ ID NO:2. In certain preferred embodiments, the nucleotide sequences are about 75 percent, or about 80 percent, or about 85 percent, or about 90 percent, or about 95, 96, 97, 98, or 99 percent identical to the nucleotide sequence as shown in SEQ ID NO:1, or the nucleotide sequences encode a polypeptide that is about 75 percent, or about 80 percent, or about 85 percent, or about 90 percent, or about 95, 96, 97, 98, or 99 percent identical to the polypeptide sequence as set forth in SEQ ID NO:2.

Differences in the nucleic acid sequence may result in conservative and/or non-conservative modifications of the amino acid sequence relative to the amino acid sequence of SEQ ID NO:2.

Conservative modifications to the amino acid sequence of SEQ ID NO:2 (and the corresponding modifications to the encoding nucleotides) will produce AFTI polypeptides having functional and chemical characteristics similar to those of naturally occurring AFTI polypeptide. In contrast, substantial modifications in the functional and/or chemical characteristics of AFTI polypeptides may be accomplished by selecting substitutions in the amino acid sequence of SEQ ID NO:2 that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., 1998, Acta Physiol. Scand. Suppl. 643: 55-67; Sasaki et al., 1998, Adv. Biophys. 35: 1-24, which discuss alanine scanning mutagenesis).

Desired amino acid substitutions (whether conservative or non-conservative) can be determined by those skilled in the art at the time such substitutions are desired. For example, amino acid substitutions can be used to identify important residues of the AFTI polypeptide, or to increase or decrease the affinity of the AFTI polypeptides described herein. Exemplary amino acid substitutions are set forth in Table I.

TABLE
Amino Acid Substitutions

| Original Residues | Exemplary Substitutions | Preferred Substitutions |
|---|---|---|
| Ala | Val, Leu, Ile | Val |
| Arg | Lys, Gln, Asn | Lys |
| Asn | Gln | Gin |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, Val, Met, | Ile |
| | Ala, Phe | |
| Lys | Arg, 1,4 Diamino- | Arg |
| | butyric Acid, Gln, Asn | |
| Met | Leu, Phe, Ile | Leu |
| Phe | Leu, Val, lie, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

In certain embodiments, conservative amino acid substitutions also encompass non-naturally occurring amino acid residues that are typically incorporated by chemical peptide synthesis rather than by synthesis in biological systems. These include peptidomimetics, and other reversed or inverted forms of amino acid moieties.

Naturally occurring residues may be divided into classes based on common side chain properties:

(1) hydrophobic: norleucine, Met, Ala, Val, Leu, Ile;

(2) neutral hydrophilic: Cys, Ser, Thr, Asn, Gin; 10 (3) acidic: Asp, Glu;

(4) basic: His, Lys, Arg;

(5) residues that influence chain orientation: Gly, Pro; and

(6) aromatic: Trp, Tyr, Phe.

For example, non-conservative substitutions may involve the exchange of a member of one of these classes for a member from another class. Such substituted residues may be introduced into regions of the human AFTI polypeptide that are homologous with non-human AFTI polypeptide orthologs, or into the non-homologous regions of the molecule.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157: 105-131 (1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those that are within ±1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (-0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred. One may also identify epitopes from primary amino acid sequences on the basis of hydrophilicity. These regions are also referred to as “epitopic core regions.”

A skilled artisan will be able to determine suitable variants of the polypeptide as set forth in SEQ ID NO:2 using well known techniques. For identifying suitable areas of the molecule that may be changed without destroying activity, one skilled in the art may target areas not believed to be important for activity. For example, when similar polypeptides with similar activities from the same species or from other species are known, one skilled in the art may compare the amino acid sequence of an AFTI polypeptide to such similar polypeptides. With such a comparison, one can identify residues and portions of the molecules that are conserved among similar polypeptides. It will be appreciated that changes in areas of an AFTI polypeptide that are not conserved relative to such similar polypeptides would be less likely to adversely affect the biological activity and/or structure of the AFTI polypeptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in an AFTI polypeptide that correspond to amino acid residues that are important for activity or structure in similar polypeptides. One skilled in the art may opt for chemically similar amino acid substitutions for such predicted important amino acid residues of AFTI polypeptides.

One skilled in the art can also analyze the three-dimensional structure and amino acid sequence in relation to that structure in similar polypeptides. In view of that information, one skilled in the art may predict the alignment of amino acid residues of an AFTI polypeptide with respect to its three dimensional structure. One skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays know to those skilled in the art. Such variants could be used to gather information about suitable variants. For example, if one discovered that a change to a particular amino acid residue resulted in destroyed, undesirably reduced, or unsuitable activity, variants with such a change would be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult J., Curr. Op. in Biotech., 7(4): 422-427 (1996), Chou et al., Biochemistry, 13(2: 222-245 (1974); Chou et al., Biochemistry, 113(2): 211-222 (1974); Chou et al., Adv. Enzymol. Relat. Areas Mol. Biol., 47: 45-148 (1978); Chou et al., Ann. Rev. Biochem., 47: 251-276 and Chou et al., Biophys. J., 26: 367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure. See Holm et al., Nucl. Acid. Res., 27(1): 244-247 (1999). It has been suggested (Brenner et al., Curr. Op. Struct. Biol., 7(3): 369-376 (1997)) that there are a limited number of folds in a given polypeptide or protein and that once a critical number of structures have been resolved, structural prediction will gain dramatically in accuracy.

Additional methods of predicting secondary structure include “threading” (Jones, D., Curr. Opin. Struct. Biol., 7(3): 377-87 (1997); Sippl et al., Structure, 4(1): 15-9 (1996)), “profile analysis” (Bowie et al., Science, 253: 164-170 (1991); Gribskov et al., Meth. Enzymol., 183: 146-159 (1990); Gribskov et al., Proc. Nat Acad. Sci., 84(13): 4355-4358 (1987)), and “evolutionary linkage” (See Home, supra, and Brenner, supra).

Identity and similarity of related nucleic acid molecules and polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, N.J., 1994; Sequence Analysis in Molecular Biology, von Heinje, G., Academic Press, 1987; Sequence Analysis Primer, Gribskov, M. and Devereux, J., eds., M. Stockton Press, N.Y., 1991; and Carillo et al., SIAM J. Applied Math., 48: 1073 (1988).

Preferred methods to determine identity and/or similarity are designed to give the largest match between the sequences tested. Methods to determine identity and similarity are described in publicly available computer programs. Preferred computer program methods to determine identity and similarity between two sequences include, but are not limited to, the GCG program package, including GAP (Devereux et al., Nucl. Acid. Res., 12: 387 (1984); Genetics Computer Group, University of Wisconsin, Madison, Wis.), BLASTP, BLASTN, and FASTA (Altschul et al., J. Mol. Biol., 215: 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al, supra). The well known Smith Waterman algorithm may also be used to determine identity.

Certain alignment schemes for aligning two amino acid sequences may result in the matching of only a short region of the two sequences, and this small aligned region may have very high sequence identity even though there is no significant relationship between the two full length sequences. Accordingly, in a preferred embodiment, the selected alignment method (GAP program) will result in an alignment that spans at least 50 contiguous amino acids of the target polypeptide.

For example, using the computer algorithm GAP (Genetics Computer Group, University of Wisconsin, Madison, Wis., two polypeptides for which the percent sequence polypeptide identity is to be determined are aligned for optimal matching of their respective amino acids (the “matched span”, as determined by the algorithm). A gap opening penalty (which is calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp.3 (1978) for the PAM 250 comparison matrix; Henikoff et al., Proc. Natl. Acad. Sci USA, 89: 10915-10919 (1992) for the BLOSUM 62 comparison matrix) is also used by the algorithm.

Preferred parameters for a polypeptide sequence comparison include the following:

Algorithm: Needleman et al., J. Mol. Biol., 48: 443-453 (1970);

Comparison matrix: BLOSUM 62 from Henikoff et al., Proc. Natl. Acad. Sci. USA, 89: 10915-10919 (1992);

Gap Penalty: 12

Gap Length Penalty: 4

Threshold of Similarity: 0

The GAP program is useful with the above parameters. The aforementioned parameters are the default parameters for polypeptide comparisons (along with no penalty for end gaps) using the GAP algorithm.

Preferred parameters for nucleic acid molecule sequence comparisons include the following:

Algorithm: Needleman et ai, J. Mol Biol., 48: 443-453 (1970);

Comparison matrix: matches=+10, mismatch=0

Gap Penalty: 50

Gap Length Penalty: 3

The GAP program is also useful with the above parameters. The aforementioned parameters are the default parameters for nucleic acid molecule comparisons.

Other exemplary algorithms, gap opening penalties, gap extension penalties, comparison matrices, thresholds of similarity, etc. may be used,, including those set forth in the Program Manual, Wisconsin Package, Version 9, September, 1997. The particular choices to be made will be apparent to those of skill in the art and will depend on the specific comparison to be made, such as DNA to DNA, protein to protein, protein to DNA; and additionally, whether the comparison is between given pairs of sequences (in which case GAP or BestFit are generally preferred) or between one sequence and a large database of sequences (in which case FASTA or BLASTA are preferred).

Preferred AFTI polypeptide variants include glycosylation variants wherein the number and/or type of glycosylation sites has been altered compared to the amino acid sequence set forth in SEQ ID NO:2. In one embodiment, AFT1 polypeptide variants comprise a greater or a lesser number of N-linked glycosylation sites than the amino acid sequence set forth in SEQ ID NO:2. An N-linked glycosylation site is characterized by the sequence: Asn—X—Ser or Asn—X—Thr, wherein the amino acid residue designated as X may be any amino acid residue except proline. The substitution(s) of amino acid residues to create this sequence provides a potential new site for the addition of an N-linked carbohydrate chain. Alternatively, substitutions that eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred AFTI variants include cysteine variants, wherein one or more cysteine residues are deleted from or substituted for another amino acid (e.g., serine) as compared to the amino acid sequence set forth in SEQ ID NO:2 or a fragment thereof. Cysteine variants are useful when AFTI polypeptides must be refolded into a biologically active conformation such as after the isolation of insoluble inclusion bodies. Cysteine variants generally have fewer cysteine residues than the native protein, and typically have an even number to minimize interactions resulting from unpaired cysteines. Additional AFTI polypeptide variants include lipidation variants, wherein a lipidation site has been added by modification (e.g., insertion or replacement of residues) of the AFTI polypeptide sequence. For such molecules, a protein of SEQ ID NO:2 could be mutated to include an amino acid or motif recognized by a lipidating enzyme (e.g., a motif recognized by farnesyl transferase and/or geranylgeranyl transferase including the CAAX motif. See, Fu et al., 1999, Recent Prog. Horm. Res. 54: 315-342; Wilson et al., 1998, Biochem. J. 333: 497-504; Khosravi-Far et al., 1992, J. Biol. Chem. 267: 24363-24368. Other lipidation techniques known in the art can also be used to modify an AFTI polypeptide of the invention.

In addition, the polypeptide comprising the amino acid sequence of SEQ ID NO:2 or an AFTi polypeptide variant may be fused to a homologous polypeptide to form a homodimer or to a heterologous polypeptide to form a heterodimer. Heterologous peptides and polypeptides include, but are not limited to: an epitope to allow for the detection and/or isolation of an AFTI fusion polypeptide; a transmembrane receptor protein or a portion thereof, such as an extracellular domain, or a transmembrane and intracellular domain; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region; and a polypeptide which has a therapeutic activity different from the polypeptide comprising the amino acid sequence as set forth in SEQ ID NO:2 or an AFTi polypeptide variant.

Fusions can be made either at the amino terminus or at the carboxy terminus of the polypeptide comprising the amino acid sequence set forth in SEQ ID NO:2 or an AFTI polypeptide variant. Fusions may be direct with no linker or adapter molecule or indirect using a linker or adapter molecule. A linker or adapter molecule may be one or more amino acid residues, typically up to about 20 to about 50 amino acid residues. A linker or adapter molecule may also be designed with a cleavage site for a DNA restriction endonuclease or for a protease to allow for the separation of the fused moieties. It will be appreciated that once constructed, the fusion polypeptides can be derivatized according to the methods described herein.

In a further embodiment of the invention, the polypeptide comprising the amino acid sequence of SEQ ID NO:2 or an AFTI polypeptide variant is fused to one or more domains of an Fc region of human IgG. Antibodies comprise two functionally independent parts, a variable domain known as “Fab”, which binds antigen, and a constant domain known as “Fc”, which is involved in effector functions such as complement activation and attack by phagocytic cells. An Fc has a long serum half-life, whereas an Fab is short-lived. Capon et al., Nature, 337: 525-31 (1989). When constructed together with a therapeutic protein, an Fc domain can provide longer half-life or incorporate such functions as Fc receptor binding, protein A binding, complement fixation and perhaps even placental transfer. Capon et al., supra. Table II summarizes the use of certain Fc fusions known in the art.

TABLE II
Fc Fusion with Therapeutic Proteins

| | Fusion | Therapeutic | |
|---|---|---|---|
| Form of Fc | partner | implications | Reference |
| lgG1 | N-terminus of | Hodgkin's disease; | U.S. Pat. |
| | CD30-L | anaplastic lymphoma; T- | No. 5,480,981 |
| | | cell leukemia | |
| Murine Fc2a | IL-10 | anti- | Zheng et al. (1995), |
| | | inflammatoy; | J. Immunol., 154: |
| | | transplant | 5590-5600 |
| | | rejection | |
| lgG1 | TNF receptor | septic shock | Fisher et al. (1996), N. Engl. |
| | | | J. Med., 334: 1697-1702; |
| | | | Van Zee et al.., (1996), J. |
| | | | Immunol., 156: 2221-2230 |
| IgG, IgA, IgM, or | TNF receptor | inflammation, autoimmune | U.S. Pat. No. 5,808,029, |
| IgE (excluding the | | disorders | issued Sep. 15, 1998 |
| first domain) | | | |
| IgG1 | CD4 receptor | AIDS | Capon et al. (1989), |
| | | | Nature 337: 525-531 |
| IgG1, IgG3 | N- | anti-cancer, antiviral | Harvill et al.. (1995), |
| IG23 | terminus | | Immunotech., V. 95-105 |
| | of IL-2 | | |
| IgG1 | C-terminus of | osteoarthritis; | WO 97/23614, published |
| | OPG | bone density | Jul. 3, 1997 |
| IgG1 | N-terminus of | anti-obesity | PCT/US 97/23183, |
| | leptin | | filed Dec. 11, 1997 |
| Human Ig C1 | CTLA-4 | autoimmune disorders | Linsley (1991), J. |
| | | | Exp. Med., 174:561-569 |

In one example, all or a portion of the human IgG hinge, CH2 and CH3 regions may be fused at either the N-terminus or C-terminus of the AFTI polypeptides using methods known to the skilled artisan. The resulting AFTI fusion polypeptide may be purified by use of a Protein A affinity column. Peptides and proteins fused to an Fc region have been found to exhibit a substantially greater half-life in vivo than the unfused counterpart. Also, a fusion to an Fc region allows for dimerization/multimerization of the fusion polypeptide. The Fc region may be a naturally occurring Fc region, or may be altered to improve certain qualities, such as therapeutic qualities, circulation time, reduce aggregation, etc.

Synthesis

It will be appreciated by those skilled in the art that the nucleic acid and polypeptide molecules described herein may be produced by recombinant and other methods.

Synthesis of Nucleic Acid Molecules

The nucleic acid molecules that encode a polypeptide comprising the amino acid sequence of an AFTI polypeptide can readily be obtained in a variety of ways including, without limitation, chemical synthesis, cDNA or genomic library screening, expression polypeptidelibrary screening and/or PCR amplification of cDNA.

Recombinant DNA methods used herein are generally those set forth in Sambrook et al., Molecular Cloning: A Laboratory Manual, Cold Spring Harbor Laboratory Press, Cold Spring Harbor, N.Y. (1989), and/or Ausubel et al., eds., Current Protocols in Molecular Biology, Green Publishers Inc. and Wiley and Sons, N.Y. (1994).The present invention provides for nucleic acid molecules as described herein and methods for obtaining the molecules.

Where a gene encoding the amino acid sequence of an AFTI polypeptide has been identified from one species, all or a portion of that gene may be used as a probe to identify orthologs or related genes from the same species. The probes or primers may be used to screen cDNA libraries from various tissue sources believed to express the AFTI polypeptide. In addition, part or all of a nucleic acid molecule having the sequence as set forth in SEQ ID NO:1 may be used to screen a genomic library to identify and isolate a gene encoding the amino acid sequence of an AFTI polypeptide. Typically, conditions of moderate or high stringency will be employed for screening to minimize the number of false positives obtained from the screen.

Nucleic acid molecules encoding the amino acid sequence of AFTI polypeptides may also be identified by expression cloning which employs the detection of positive clones based upon a property of the expressed protein. Typically, nucleic acid libraries are screened by the binding of an antibody or other binding partner (e.g., receptor or ligand) to cloned proteins that are expressed and displayed on a host cell surface. The antibody or binding partner is modified with a detectable label to identify those cells expressing the desired clone.

Recombinant expression techniques conducted in accordance with the descriptions set forth below may be followed to produce these polynudeotides and to express the encoded polypeptides. For example, by inserting a nucleic acid sequence that encodes the amino acid sequence of an AFTI polypeptide into an appropriate vector, one skilled in the art can readily produce large quantities of the desired nudeotide sequence. The sequences can then be used to generate detection probes or amplification primers. Alternatively, a polynucleotide encoding the amino acid sequence of an AFTI polypeptide can be inserted into an expression vector. By introducing the expression vector into an appropriate host, the encoded AFTI polypeptide may be produced in large amounts.

Another method for obtaining a suitable nucleic acid sequence is the polymerase chain reaction (PCR). In this method, cDNA is prepared from poly(A)+RNA or total RNA using the enzyme reverse transcriptase. Two primers, typically complementary to two separate regions of cDNA (oligonucleotides) encoding the amino acid sequence of an AFTI polypeptide, are then added to the cDNA along with a polymerase such as Taq polymerase, and the polymerase amplifies the cDNA region between the two primers.

Another means of preparing a nucleic acid molecule encoding the amino acid sequence of an AFTI polypeptide is chemical synthesis using methods well known to the skilled artisan such as those described by Engels et al., Angew. Chem. Intl. Ed., 28: 716-734 (1989). These methods include, inter alia, the phosphotriester, phosphoramidite, and H-phosphonate methods for nucleic acid synthesis. A preferred method for such chemical synthesis is polymer-supported synthesis using standard phosphoramidite chemistry.

Typically, the DNA encoding the amino acid sequence of an AFTI polypeptide will be several hundred nucleotides in length. Nucleic acids larger than about 100 nucleotides can be synthesized as several fragments using these methods. The fragments can then be ligated together to form the full length nudeotide sequence of an AFTI polypeptide. Usually, the DNA fragment encoding the amino terminus of the polypeptide will have an ATG, which encodes a methionine residue. This methionine may or may not be present on the mature form of the AFTI polypeptide, depending on whether the polypeptide produced in the host cell is designed to be secreted from that cell. Other methods known to the skilled artisan may be used as well.

In certain embodiments, nucleic acid variants contain codons that have been altered for the optimal expression of an AFTI polypeptide in a given host cell. Particular codon alterations will depend upon the AFTI polypeptide(s) and host cell(s) selected for expression. Such “codon optimization” can be carried out by a variety of methods, for example, by selecting codons that are preferred for use in highly expressed genes in a given host cell. Computer algorithms that incorporate codon frequency tables such as “Ecohigh.cod” for codon preference of highly expressed bacterial genes may be used and are provided by the University of Wisconsin Package Version 9.0, Genetics Computer Group, Madison, Wis.. Other useful codon frequency tables include “Celegan_shigh.cod”, “Celegans_low.cod”, “Drosophila_high.cod”, “Human_high.cod”, “Maize_high.cod”, and “Yeast_high.cod”.

Vectors and Host Cells

A nucleic acid molecule encoding the amino acid sequence of an AFTI polypeptide may be inserted into an appropriate expression vector using standard ligation techniques. The vector is typically selected to be functional in the particular host cell employed (i.e., the vector is compatible with the host cell machinery such that amplification of the gene and/or expression of the gene can occur). A nucleic acid molecule encoding the amino acid sequence of an AFTI polypeptide may be amplified/expressed in prokaryotic, yeast, insect (baculovirus systems), and/or eukaryotic host cells. Selection of the host cell will depend in part on whether an AFTI polypeptide is to be post-translationally modified (e.g., glycosylated and/or phosphorylated). If so, yeast, insect, or mammalian host cells are preferable. For a review of expression vectors, see Meth. Enzymol., v.185, D. V. Goeddel, ed. Academic Press Inc., San Diego, Calif. (1990).

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oiigonucleotide molecule located at the 5 or 3′ end of the AFTI polypeptide coding sequence; the oligonucleotide sequence encodes polyHis (such as hexaHis), or other polypeptide “tag” such as FLAG, HA (hemaglutinin Influenza virus) or myc for which commercially available antibodies exist. This tag is typically fused to the polypeptide upon expression of the polypeptide, and can serve as a means for affinity purification of the AFTI polypeptide from the host cell. Affinity purification can be accomplished, for example, by column chromatography using antibodies against the tag as an affinity matrix. Optionally, the tag can subsequently be removed from the purified AFTI polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source) or synthetic, or the flanking sequences may be native sequences which normally function to regulate AFTI polypeptide expression. As such, the source of a flanking sequence may be any prokaryotic or eukaryotic organism, any vertebrate or invertebrate organism, or any plant, provided that the flanking sequence is functional in, and can be activated by, the host cell machinery.

The flanking sequences useful in the vectors of this invention may be obtained by any of several methods well known in the art. Typically, flanking sequences useful herein other than the AFTI gene flanking sequences will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Where all or only a portion of the flanking sequence is known, it may be obtained using PCR and/or by screening a genomic library with suitable oligonucleotide and/or flanking sequence fragments from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. Amplification of the vector to a certain copy number can, in some cases, be important for the optimal expression of an AFTI polypeptide. If the vector of choice does not contain an origin of replication site, one may be chemically synthesized based on a known sequence, and ligated into the vector. For example, the origin of replication from the plasmid pBR322 (Product No. 303-3s, New England Biolabs, Beverly, Mass.) is suitable for most Gram-negative bacteria and various origins (e.g., SV40, polyoma, adenovirus, vesicular stomatitus virus (VSV) or papillomaviruses such as HPV or BPV) are useful for cloning vectors in mammalian cells. Generally, the origin of replication component is not needed for mammalian expression vectors (for example, the SV40 origin is often used only because it contains the early promoter).

A transcription termination sequence is typically located 3′ of the end of a polypeptide coding region and serves to terminate transcription. Usually, a transcription termination sequence in prokaryotic cells is a G-C rich fragment followed by a poly T sequence. While the sequence is easily cloned from a library or even purchased commercially as part of a vector, it can also be readily synthesized using methods for nucleic acid synthesis such as those described herein.

A selectable marker gene element encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells.

Other selection genes may be used to amplify the gene that will be expressed. Amplification is the process wherein genes that are in greater demand for the production of a protein critical for growth are reiterated in tandem within the chromosomes of successive generations of recombinant cells. Examples of suitable selectable markers for mammalian cells include dihydrofolate reductase (DHFR) and thymidine kinase. The mammalian cell transformants are placed under selection pressure that only the transformants are uniquely adapted to survive by virtue of the selection gene present in the vector. Selection pressure is imposed by culturing the transformed cells under conditions in which the concentration of selection agent in the medium is successively changed, thereby leading to the amplification of both the selection gene and the DNA that encodes an AFTI polypeptide. As a result, increased quantities of AFTI polypeptide are synthesized from the amplified DNA.

A ribosome binding site is usually necessary for translation initiation of mRNA and is characterized by a Shine-Dalgamo sequence (prokaryotes) or a Kozak sequence (eukaryotes). The element is typically located 3′ to the promoter and 5′ to the coding sequence of an AFTI polypeptide to be expressed. The Shine-Dalgamo sequence is varied but is typically a polypurine (i.e., having a high A-G content). Many Shine-Dalgamo sequences have been identified, each of which can be readily synthesized using methods set forth herein and used in a prokaryotic vector.

A leader, or signal, sequence may be used to direct an AFTI polypeptide out of the host cell. Typically, a nucleotide sequence encoding the signal sequence is positioned in the coding region of an AFTI nucleic acid molecule, or directly at the 5 end of an AFTI polypeptide coding region. Many signal sequences have been identified, and any of those that are functional in the selected host cell may be used in conjunction with an AFTI nucleic acid molecule. Therefore, a signal sequence may be homologous (naturally occurring) or heterologous to an AFTI gene or cDNA. Additionally, a signal sequence may be chemically synthesized using methods described herein. In most cases, the secretion of an AFTI polypeptide from the host cell via the presence of a signal peptide will result in the removal of the signal peptide from the secreted AFTI polypeptide. The signal sequence may be a component of the vector, or it may be a part of an AFTI nucleic acid molecule that is inserted into the vector.

Included within the scope of this invention is the use of either a nucleotide sequence encoding a native AFTI polypeptide signal sequence joined to an AFTI polypeptide coding region or a nucleotide sequence encoding a heterologous signal sequence joined to an AFTI polypeptide coding region. The heterologous signal sequence selected should be one that is recognized and processed, i.e., cleaved by a signal peptidase, by the host cell. For prokaryotic host cells that do not recognize and process the native AFTI polypeptide signal sequence, the signal sequence is substituted by a prokaryotic signal sequence selected, for example, from the group of the alkaline phosphatase, penicillinase, or heat-stable enterotoxin II leaders. For yeast secretion, the native AFTI polypeptide signal sequence may be substituted by the yeast invertase, alpha factor, or acid phosphatase leaders. In mammalian cell expression the native signal sequence is satisfactory, although other mammalian signal sequences may be suitable.

In some cases, such as where glycosylation is desired in a eukaryotic host cell expression system, one may manipulate the various presequences to improve glycosylation or yield. For example, one may alter the peptidase cleavage site of a particular signal peptide, or add presequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the N-terminus. Alternatively, use of some enzyme cleavage sites may result in a slightly truncated form of the desired AFTI polypeptide, if the enzyme cuts at such area within the mature polypeptide.

In many cases, transcription of a nucleic acid molecule is increased by the presence of one or more introns in the vector; this is particularly true where a polypeptide is produced in eukaryotic host cells, especially mammalian host cells. The introns used may be naturally occurring within the AFTI gene, especially where the gene used is a full length genomic sequence or a fragment thereof. Where the intron is not naturally occurring within the gene (as for most cDNAs), the intron(s) may be obtained from another source. The position of the intron with respect to flanking sequences and the AFTI gene is generally important, as the intron must be transcribed to be effective. Thus, when an AFTI cDNA molecule is being transcribed, the preferred position for the intron is 3 to the transcription start site, and 5′ to the polyA transcription termination sequence. Preferably, the intron or introns will be located on one side or the other (i.e., 5′ or 3′) of the cDNA such that it does not interrupt the coding sequence. Any intron from any source, including any viral, prokaryotic and eukaryotic (plant or animal) organisms, may be used to practice this invention, provided that it is compatible with the host cell(s) into which it is inserted. Also included herein are synthetic introns. Optionally, more than one intron may be used in the vector.

The expression and cloning vectors of the present invention will each typically contain a promoter that is recognized by the host organism and operably linked to the molecule encoding a AFTI polypeptide. Promoters are untranscribed sequences located upstream (5′) to the start codon of a structural gene (generally within about 100 to 1000 bp) that control the transcription of the structural gene. Promoters are conventionally grouped into one of two classes, inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding an AFTI polypeptide by removing the promoter from the source DNA by restriction enzyme digestion and inserting the desired promoter sequence into the vector. The native AFTI gene promoter sequence may be used to direct amplification and/or expression of an AFTI nucleic acid molecule. A heterologous promoter is preferred, however, if it permits greater transcription and higher yields of the expressed protein as compared to the native promoter, and if it is compatible with the host cell system that has been selected for use.

Promoters suitable for use with prokaryotic hosts include the beta-lactamase and lactose promoter systems; alkaline phosphatase, a tryptophan (trp) promoter system; and hybrid promoters such as the tac promoter. Other known bacterial promoters are also suitable. Their sequences have been published, thereby enabling one skilled in the art to ligate them to the desired DNA sequence(s), using linkers or adapters as needed to supply any useful restriction sites.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus (CMV), a retrovirus, hepatitis-B virus and most preferably Simian Virus 40 (SV40).Other suitable mammalian promoters include heterologous mammalian promoters, e.g., heat-shock promoters and the actin promoter.

Additional promoters that may be of interest in controlling AFT1 gene transcription include, but are not limited to: the SV40 early promoter region (Bemoist and Chambon, Nature, 290: 304-310, 1981); the CMV promoter; the promoter contained in the 3′ long terminal repeat of Rous sarcoma virus (Yamamoto et al., Cell, 22: 787-797, 1980); the herpes thymidine kinase promoter (Wagner et al., Proc. Natl. Acad. Sci. USA, 78: 144-1445, 1981); the regulatory sequences of the metallothionine gene (Brinster et al., Nature, 296: 39-42, 1982); for prokaryotic expression vectors promoters such as the beta-lactamase promoter (Villa-Kamaroff, et al., Proc. Natl. Acad. Sci. USA, 75: 3727-3731, 1978); or the tac promoter (DeBoer, et al., Proc. Natl. Acad. Sci. USA, 80: 21-25, 1983). Also of interest are the following animal transcriptional control regions, which exhibit tissue specificity and have been utilized in transgenic animals: the elastase I gene control region which is active in pancreatic acinar cells (Swift et al., Cell, 38: 639-646, 1984; Omitz et al., Cold Spring Harbor Symp. Quant. Biol., 50: 399-409 (1986); MacDonald, Hepatology, 7: 425-515, 1987); the insulin gene control region which is active in pancreatic beta cells (Hanahan, Nature, 315: 115-122, 1985); the immunoglobulin gene control region which is active in lymphoid celis (Grosschedl et al., Cell, 38: 647-658 (1984); Adames et al., Nature, 318: 533-538 (1985); Alexander et al, Mol. Cell. Biol., 7: 1436-1444, 1987); the mouse mammary tumor virus control region which is active in testicular, breast, lymphoid and mast cells (Leder et al., Cell, 45: 485-495, 1986); the albumin gene control region which is active in liver (Pinkert et al., Genes and Devel, 1: 268-276, 1987); the alphafetoprotein gene control region which is active in liver (Krumlauf et al., Mol. Cell. Biol., 5: 1639-1648, 1985; Hammer et al., Science, 235: 53-58, 1987); the alpha 1-antitrypsin gene control region which is active in the liver (Kelsey et al., Genes and Devel., 1: 161-171, 1987); the beta-globin gene control region which is active in myeloid cells (Mogram et al., Nature, 315: 338-340, 1985; Kollias et al., Cell, 46: 89-94, 1986); the myelin basic protein gene control region which is active in oligodendrocyte cells in the brain (Readhead et al., Cell, 48: 703-712, 1987); the myosin light chain-2 gene control region which is active in skeletal muscle (Sani, Nature, 314: 283-286, 1985); and the gonadotropin releasing hormone gene control region which is active in the hypothalamus (Mason et al., Science, 234: 1372-1378, 1986).

An enhancer sequence may be inserted into the vector to increase the transcription of a DNA encoding an AFTI polypeptide of the present invention by higher eukaryotes. Enhancers are cis-acting elements of DNA, usually about 10-300 bp in length, that act on the promoter to increase transcription. Enhancers are relatively orientation and position independent. They have been found 5′ and 3′ to the transcription unit. Several enhancer sequences available from mammalian genes are known (e.g., globin, elastase, albumin, alpha-feto-protein and insulin). Typically, however, an enhancer from a virus will be used. The SV40 enhancer, the cytomegalovirus early promoter enhancer, the polyoma enhancer, and adenovirus enhancers are exemplary enhancing elements for trie activation of eukaryotic promoters. While an enhancer may be spliced into the vector at a position 5′ or 3′ to an AFTI nucleic acid molecule, it is typically located at a site 5 from the promoter.

Expression vectors of the invention may be constructed from a starting vector such as a commercially available vector. Such vectors may or may not contain all of the desired flanking sequences. Where one or more of the desired flanking sequences are not already present in the vector, they may be individually obtained and ligated into the vector. Methods used for obtaining each of the flanking sequences are well known to one skilled in the art.

Preferred vectors for practicing this invention are those that are compatible with bacterial, insect, yeast, and mammalian host cells. Such vectors include, inter alia, pCRII, pCR3, and pcDNA3.1 (Invitrogen Company, Carlsbad, Calif.), pBSII (Stratagene Company, La Jolla, Calif.), pET15b (Novagen, Madison, Wis.), pGEX (Pharmacia Biotech, Piscataway, N.J.), pEGFP-N2 (Clontech, Palo Alto, Calif.), pETL (BlueBacll; Invitrogen), pDSR-alpha (PCT Publication No. WO90/14363) and pFastBacDual (Gibco/BRL, Grand polypeptideIsland, N.Y.).

Additional suitable vectors include, but are not limited to, cosmids, plasmids or modified viruses, but it will be appreciated that the vector system must be compatible with the selected host cell. Such vectors include, but are not limited to plasmids such as Bluescript® plasmid derivatives (a high copy number ColE1-based phagemid, Stratagene Cloning Systems Inc., La Jolla Calif.), PCR cloning plasmids designed for cloning Taq-amplified PCR products (e.g., TOPO™ TA Cloning® Kit, PCR2.1® plasmid derivatives, Invitrogen, Carlsbad, Calif.), and mammalian, yeast, insect, or virus vectors such as a baculovirus expression system (pBacPAK plasmid derivatives, Clontech, Palo Alto, Calif.).

After the vector has been constructed and a nucleic acid molecule encoding an AFT1 polypeptide has been inserted into the proper site of the vector, the completed vector may be inserted into a suitable host cell for amplification and/or polypeptide expression. The transformation of an expression vector for an AFT1 polypeptide into a selected host cell may be accomplished by well known methods including methods such as transfection, infection, calcium chloride, electroporation, microinjection, lipofection or the DEAE-dextran method or other known techniques. The method selected will in part be a function of the type of host cell to be used. These methods and other suitable methods are well known to the skilled artisan, and are set forth, for example, in Sambrook et al., supra.

Host cells may be prokaryotic host cells (such as E. coli) or eukaryotic host cells (such as a yeast cell, an insect cell or a vertebrate cell). The host cell, when cultured under appropriate conditions, synthesizes an AFTI polypeptide which can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity, such as glycosylation or phosphorylation, and ease of folding into a biologically active molecule.

A number of suitable host cells are known in the art and many are available from the American Type Culture Collection (ATCC), 10801 University Boulevard, Manassas, Va. 20110-2209. Examples include, but are not limited to, mammalian cells, such as Chinese hamster ovary cells (CHO) (ATCC No. CCL61) CHO DHFR-cells (Urlaub et al., Proc. Natl. Acad. Sci. USA, 97: 4216-4220 (1980)), human embryonic kidney (HEK) 293 or 293T cells (ATCC No. CRL1573), or 3T3 cells (ATCC No. CCL92). The selection of suitable mammalian host cells and methods for transformation, culture, amplification, screening and product production and purification are known in the art. Other suitable mammalian cell lines, are the monkey COS-1 (ATCC No. CRL1650) and COS-7 cell lines (ATCC No. CRL1651), and the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection gene, or may contain a dominantly acting selection gene. Other suitable mammalian cell lines include but are not limited to, mouse neuroblastoma N2A cells, HeLa, mouse L-929 cells, 3T3 lines derived from Swiss, Balb-c or NIH mice, BHK or HaK hamster cell lines, which are available from the ATCC. Each of these cell lines is known by and available to those skilled in the art of protein expression.

Similarly useful as host cells suitable for the present invention are bacterial cells. For example, the various strains of E. coli (e.g., HB101, (ATCC No. 33694) DH5 α, DH10, and MC1061 (ATCC No. 53338)) are well-known as host cells in the field of biotechnology. Various strains of B. subtilis, Pseudomonas spp., other Bacillus spp. Streptomyces spp., and the like may also be employed in this method.

Many strains of yeast cells known to those skilled in the art are also available as host cells for the expression of the polypeptides of the present invention. Preferred yeast cells include, for example, Saccharomyces cerivisae and Pichia pastoris.

Additionally, where desired, insect cell systems may be utilized in the methods of the present invention. Such systems are described for example in Kitts et al., Biotechniques, 14: 810-817 (1993); Lucklow, Curr. Opin. Biotechnol., 4: 564-572 (1993); and Lucklow et al. (J. Virol., 67: 4566-4579 (1993). Preferred insect cells are Sf-9 and Hi5 (Invitrogen, Carlsbad, Calif.).

One may also use transgenic animals to express glycosylated AFTI polypeptides. For example, one may use a transgenic milk-producing animal (a cow or goat, for example) and obtain the present glycosylated polypeptide in the animal milk. One may also use plants to produce AFTI polypeptides, however, in general, the glycosylation occurring in plants is different from that produced in mammalian cells, and may result in a glycosylated product that is not suitable for human therapeutic use.

Polypeptide Production

Host cells comprising an AFTI polypeptide expression vector may be cultured using standard media well known to the skilled artisan. The media will usually contain all nutrients necessary for the growth and survival of the cells. Suitable media for culturing E. coli cells include, for example, Luria Broth (LB) and/or Terrific Broth (TB). Suitable media for culturing eukaryotic cells include Roswell Park Memorial Institute medium 1640 (RPMI 1640), Minimal Essential Medium (MEM) and/or Dulbecco's Modified Eagle Medium (DMEM), all of which may be supplemented with serum and/or growth factors as indicated by the particular cell line being cultured. A suitable medium for insect cultures is Grace's medium supplemented with yeastolate, lactalbumin hydrolysate and/or fetal calf serum, as necessary.

Typically, an antibiotic or other compound useful for selective growth of transformed cells is added as a supplement to the media. The compound to be used will be dictated by the selectable marker element present on the plasmid with which the host cell was transformed. For example, where the selectable marker element is kanamycin resistance, the compound added to the culture medium will be kanamycin. Other compounds for selective growth include ampicillin, tetracycline, and neomycin.

The amount of an AFTI polypeptide produced by a host cell can be evaluated using standard methods known in the art. Such methods include, without limitation, Western blot analysis, SDS-polyacrylamide gel electrophoresis, non-denaturing gel electrophoresis, HPLC separation, immunoprecipitation, and/or activity assays such as DNA binding gel shift assays.

If an AFTI polypeptide has been designed to be secreted from the host cells, the majority of polypeptide may be found in the cell culture medium. If however, the AFTI polypeptide is not secreted from the host cells, it will be present in the cytoplasm and/or the nucleus (for eukaryotic host cells) or in the cytosol (for bacterial host cells).

For an AFTI polypeptide situated in the host cell cytoplasm and/or the nucleus (for eukaryotic host cells) or in the cytosol (for bacterial host cells), intracellular material (including inclusion bodies for gram-negative bacteria) can be extracted from the host cell using any standard technique known to the skilled artisan. For example, the host cells can be lysed to release the contents of the periplasm/cytopiasm by French press, homogenization, and/or sontcation followed by centrifugation.

If an AFTI polypeptide has formed inclusion bodies in the cytosol, the inclusion bodies can often bind to the inner and/or outer cellular membranes and thus will be found primarily in the pellet material after centrifugation. The pellet material can then be treated at pH extremes or with a chaotropic agent such as a detergent, guanidine, guanidine derivatives, urea, or urea derivatives in the presence of a reducing agent such as dithiothreitol at alkaline pH or Tris carboxyethyl phosphine at acid pH to release, break apart, and solubilize the inclusion bodies. The AFTI polypeptide in its now soluble form can then be analyzed using gel electrophoresis, immunoprecipitation or the like. If it is desired to isolate the AFTI polypeptide, isolation may be accomplished using standard methods such as those described herein and in Marston et al., Meth. Enzymol., 182: 264-275 (1990).

In some cases, an AFTI polypeptide may not be biologically active upon isolation. Various methods for “refolding” or converting the polypeptide to its tertiary structure and generating disulfide linkages can be used to restore biological activity. Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential allowing for disulfide shuffling to occur in the formation of the protein's cysteine bridge(s). Some of the commonly used redox couples include cysteine/cystamine, glutathione (GSH)/dithiobis GSH, cupric chloride, dithiothreitol(DTT)/dithiane DTT, and 2-2mercaptoethanol(bME)/dithio-b(ME). A cosotvent may be used to increase the efficiency of the refolding, and the more common reagents used for this purpose include glycerol, polyethylene glycol of various molecular weights, arginine and the like.

If inclusion bodies are not formed to a significant degree upon expression of an AFTI polypeptide, then the polypeptide will be found primarily in the supernatant after centrifugation of the cell homogenate. The polypeptide may be further isolated from the supernatant using methods such as those described herein.

The purification of an AFTI polypeptide from solution can be accomplished using a variety of techniques. If the polypeptide has been synthesized such that it contains a tag such as Hexahistidine (AFTI polypeptide/hexaHis) or other small peptide such as FLAG (Eastman Kodak Co., New Haven, Conn.) or myc (Invitrogen, Carlsbad, Calif.) at either its carboxyl or amino terminus, it may be purified in a one-step process by passing the solution through an affinity column where the column matrix has a high affinity for the tag.

For example, polyhistidine binds with great affinity and specificity to nickel, thus an affinity column of nickel (such as the Qiagen® nickel columns) can be used for purification of AFTI polypeptide/polyHis. See for example, Ausubel et al., eds., Current Protocols in Molecular Biology, Section 10.11.8, John Wiley & Sons, New York (1993).

Additionally, the AFTI like polypeptide may be purified through the use of a monoclonal antibody which is capable of specifically recognizing and binding to the AFTI like polypeptide.

Suitable procedures for purification thus include, without limitation, affinity chromatography, immunoaffinity chromatography, ion exchange chromatography, molecular sieve chromatography, High Performance Liquid Chromatography (HPLC), electrophoresis (including native gel electrophoresis) followed by gel elution, and preparative isoelectric focusing (“Isoprime” machine/technique, Hoefer Scientific, San Francisco, Calif.). In some cases, two or more purification techniques may be combined to achieve increased purity.

AFTI polypeptides may also be prepared by chemical synthesis methods (such as solid phase peptide synthesis) using techniques known in the art, such as those set forth by Merrifield et al. J. Am. Chem. Soc, 85: 2149 (1963), Houghten et al., Proc Natl Acad. Sci. USA, 82: 5132 (1985), and Stewart and Young, Solid Phase Peptide Synthesis, Pierce Chemical Co., Rockford, Ill. (1984). Such polypeptides may be synthesized with or without a methionine on the amino terminus. Chemically synthesized AFTI polypeptides may be oxidized using methods set forth in these references to form disulfide bridges. Chemically synthesized AFTI polypeptides are expected to have comparable biological activity to the corresponding AFTI polypeptides produced recombinantly or purified from natural sources, and thus may be used interchangeably with a recombinant or natural AFTI polypeptide.

Another means of obtaining an AFTI polypeptide is via purification from biological samples such as source tissues and/or fluids in which the AFTI polypeptide is naturally found. Such purification can be conducted using methods for protein purification as described herein. The presence of the AFTI polypeptide during purification may be monitored using, for example, an antibody prepared against recombinantly produced AFTI polypeptide or peptide fragments thereof.

A number of additional methods for producing nucleic acids and polypeptides are known in the art, and can be used to produce polypeptides having specificity for AFTI. See, for example, Roberts, et al., Proc. Natl. Acad. Sci., 94: 12297-12303 (1997), which describes the production of fusion proteins between an mRNA and its encoded peptide. See also U.S. Pat. No. 5,824,469, which describes methods of obtaining oligonucleotides capable of carrying out a specific biological function. The procedure involves generating a heterogeneous pool of oligonucleotides, each having a 5′ randomized sequence, a central preselected sequence, and a 3′ randomized sequence. The resulting heterogeneous pool is introduced into a population of cells that do not exhibit the desired biological function. Subpopulations of the cells are then screened for those that exhibit a predetermined biological function. From that subpopulation, oligonucleotides capable of carrying out the desired biological function are isolated.

U.S. Pat. Nos. 5,763,192, 5,814,476, 5,723,323, and 5,817,483 describe processes for producing peptides or polypeptides. This is done by producing stochastic genes or fragments thereof, and then introducing these genes into host cells that produce one or more proteins encoded by the stochastic genes. The host cells are then screened to identify those clones producing peptides or polypeptides having the desired activity.

Chemical Derivatives

Chemically modified derivatives of the AFTI polypeptides may be prepared by one skilled in the art, given the disclosures set forth below. AFTI polypeptide derivatives are modified in a manner that is different, either in the type or location of the molecules naturally attached to the polypeptide. Derivatives may include molecules formed by the deletion of one or more naturally-attached chemical groups. The polypeptide comprising the amino acid sequence of SEQ ID NO:2, or an AFTI polypeptide variant, may be modified by the covalent attachment of one or more polymers. For example, the polymer selected is typically water soluble so that the protein to which it is attached does not precipitate in an aqueous environment, such as a physiological environment. Included within the scope of suitable polymers is a mixture of polymers. Preferably, for therapeutic use of the end-product preparation, the polymer will be pharmaceutically acceptable.

The polymers each may be of any molecular weight and may be branched or unbranched. The polymers each typically have an average molecular weight of between about 2 kDa to about 100 kDa (the term “about” indicating that in preparations of a water soluble polymer, some molecules will weigh more, some less, than the stated molecular weight). The average molecular weight of each polymer preferably is between about kDa and about 50 kDa, more preferably between about 125 kDa and about 40 kDa and most preferably between about 20 kDa and about 35 kDa.

Suitable water soluble polymers or mixtures thereof include, but are not limited to, N-linked or O-linked carbohydrates, sugars, phosphates, polyethylene glycol (PEG) (including the forms of PEG that have been used to derivatize proteins, including mono-(C₁-C₁₀) alkoxy- or aryloxy-polyethylene glycol), monomethoxy-polyethylene glycol, dextran (such as low molecular weight dextran, of, for example about 6 kDa), cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (e.g., glycerol) and polyvinyl alcohol. Also encompassed by the present invention are bifunctional crosslinking molecules that may be used to prepare covalently attached multimers of the polypeptide comprising the amino acid sequence of SEQ ID NO:2 or an AFTI polypeptide variant.

In general, chemical derivatization may be performed under any suitable condition used to react a protein with an activated polymer molecule. Methods for preparing chemical derivatives of polypeptides will generally comprise the steps of (a) reacting the polypeptide with the activated polymer molecule (such as a reactive ester or aldehyde derivative of the polymer molecule) under conditions whereby the polypeptide comprising the amino acid sequence of SEQ ID NO:1, or an AFTI polypeptide variant, becomes attached to one or more polymer molecules, and (b) obtaining the reaction product(s). The optimal reaction conditions will be determined based on known parameters and the desired result. For example, the larger the ratio of polymer molecules: protein, the greater the percentage of attached polymer molecule. In one embodiment, the AFTI polypeptide derivative may have a single polymer molecule moiety at the amino terminus. See, for example, U.S. Pat. No. 5,234,784.

The pegylation of the polypeptide specifically may be carried out by any of the pegylation reactions known in the art, as described for example in the following references: Francis et al., Focus on Growth Factors, 3: 4-10 (1992); EP 0154316; EP 0401384 and U.S. Pat. No. 4,179,337. For example, pegylation may be carried out via an acylation reaction or an alkylation reaction with a reactive polyethylene glycol molecule (or an analogous reactive water-soluble polymer) as described herein. For the acylation reactions, the polymer(s) selected should have a single reactive ester group. For reductive alkylation, the polymer(s) selected should have a single reactive aldehyde group. A reactive aldehyde is, for example, polyethylene glycol propionaldehyde, which is water stable, or mono C₁-C₁₀ alkoxy or aryloxy derivatives thereof (see, U.S. Pat. No. 5,252,714).

In another embodiment, AFTI polypeptides may be chemically coupled to biotin, and the biotin/AFTI polypeptide molecules that are conjugated are then allowed to bind to avidin, resulting in tetravalent avidin/biotin/AFTI polypeptide molecules. AFTI polypeptides may also be covalently coupled to dinitrophenol (DNP) or trinitrophenol (TNP) and the resulting conjugates precipitated with anti-DNP or anti-TNP-IgM to form decameric conjugates with a valency of 10.

Generally, conditions that may be alleviated or modulated by the administration of the present AFTI polypeptide derivatives include those described herein for AFTI polypeptides. However, the AFTI polypeptide derivatives disclosed herein may have additional activities, enhanced or reduced biological activity, or other characteristics, such as increased or decreased half-life, as compared to the non-derivatized molecules.

Another form of chemical derivative is a lipidated AFTI polypeptide. For such molecules, one or more lipids may be covalently attached to an AFTI polypeptide or the polypeptide of SEQ ID NO:1 or a variant thereof by any means, for example, by chemical means.

Genetically Engineered Non-Human Animals

Additionally included within the scope of the present invention are non-human animals such as, for example, mice, rats, or other rodents, rabbits, goats, cows, pigs, or sheep, or other farm animals, in which the gene (or genes) encoding the native AFTI polypeptide has (have) been disrupted (“knocked out”) such that the level of expression of this gene or genes is (are) significantly decreased or completely abolished. Such animals may be prepared using techniques and methods such as those described in U.S. Pat. No. 5,557,032.

The present invention further includes non-human animals such as mice, rats, or other rodents, rabbits, goats, sheep, or other farm animals, in which either the native form of the AFTI gene(s) for that animal or a heterologous AFTI gene(s) is (are) over-expressed by the animal, thereby creating a “transgenic” animal. Such transgenic animals may be prepared using well known methods such as those described in U.S. Pat. No 5,489,743 and PCT application No. WO94/28 122.

The present invention further includes non-human animals in which the promoter for one or more of the AFTI polypeptides of the present invention is either activated or inactivated (e.g., by using homologous recombination methods) to alter the level of expression of one or more of the native AFTI polypeptides.

These non-human animals may be used for drug candidate screening. In such screening, the impact of a drug candidate on the animal may be measured. For example, drug candidates may decrease or increase the expression of the AFTI gene. In certain embodiments, the amount of AFTI polypeptide, that is produced may be measured after the exposure of the animal to the drug candidate. Additionally, in certain embodiments, one may detect the actual impact of the drug candidate on the animal. For example, the overexpression of a particular gene may result in, or be associated with, a disease or pathological condition. In such cases, one may test a drug candidate's ability to decrease expression of the gene or its ability to prevent or inhibit a pathological condition. In other examples, the production of a particular metabolic product such as a fragment of a polypeptide, may result in, or be associated with, a disease or pathological condition. In such cases, one may test a drug candidate's ability to decrease the production of such a metabolic product or its ability to prevent or inhibit a pathological condition.

Microarray

It will be appreciated that DNA microarray technology can be utilized in accordance with the present invention. DNA microarrays are miniature, high density arrays of nucleic acids positioned on a solid support, such as glass. Each cell or element within the array has numerous copies of a single species of DNA which acts as a target for hybridization for its cognate mRNA. In certain embodiments, in expression profiling using DNA microarray technology, mRNA is first extracted from a cell or tissue sample and then converted enzymatically to fluorescently labeled cDNA. This material is hybridized to the microarray and unbound cDNA is removed by washing. The expression of discrete genes represented on the array is then visualized by quantitating the amount of labeled cDNA that is specifically bound to each target DNA. In this way, the expression of thousands of genes can be quantitated in a high throughput, parallel manner from a single sample of biological material.

This high throughput expression profiling has a broad range of applications with respect to the AFTI like molecules of the invention, including, but not limited to: the identification and validation of AFTI disease-related genes as targets for therapeutics; molecular toxicology of AFTI like molecules and inhibitors thereof, stratification of populations and generation of surrogate markers for clinical trials; and enhancing AFTI-related small molecule drug discovery by aiding in the identification of selective compounds in high throughput screens (HTS).

Selective Binding Agents

As used herein, the term “selective binding agent” refers to a molecule that has binding specificity for one or more AFTI polypeptides. Suitable selective binding agents include, but are not limited to, antibodies and derivatives thereof, polypeptides, and small molecules. Suitable selective binding agents may be prepared using methods known in the art. An exemplary AFTI polypeptide selective binding agent of the present invention is capable of binding a certain portion of the AFTI polypeptide thereby inhibiting the binding of the polypeptide to the AFTI receptor(s).

Selective binding agents such as antibodies and antibody fragments that bind AFTI polypeptides are within the scope of the present invention. The antibodies may be polyclonal including monospecific polyclonal, monoclonal (MAbs), recombinant, chimeric, humanized such as CDR-grafted, human, single chain, and/or bispecific, as well as fragments, variants or derivatives thereof. Antibody fragments include those portions of the antibody that bind to an epitope on the AFTI polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Polyclonal antibodies directed toward an AFTI polypeptide generally are produced in animals (e.g., rabbits or mice) by means of multiple subcutaneous or intraperitoneal injections of AFTI polypeptide and an adjuvant. It may be useful to conjugate an AFTI polypeptide to a carrier protein that is immunogenic in the species to be immunized, such as keyhole limpet heocyanin, serum, albumin, bovine thyroglobulin, or soybean trypsin inhibitor. Also, aggregating agents such as alum are used to enhance the immune response. After immunization, the animals are bled and the serum is assayed for anti-AFTI polypeptide antibody titer.

Monoclonal antibodies directed toward an AFTI polypeptide are produced using any method that provides for the production of antibody molecules by continuous cell lines in culture. Examples of suitable methods for preparing monoclonal antibodies include the hybridoma methods of Kohler et al., Nature, 256: 495-497 (1975) and the human B-cell hybridoma method, Kozbor, J. Immunol., 133: 3001 (1984); Brodeur et al., Monoclonal Antibody Production Techniques and Applications, pp. 51-63 (Marcel Dekker, Inc., New York, 1987). Also provided by the invention are hybridoma cell lines that produce monoclonal antibodies reactive with AFTI polypeptides.

Monoclonal antibodies of the invention may be modified for use as therapeutics. One embodiment is a “chimeric” antibody in which a portion of the heavy and/or light chain is identical with or homologous to a corresponding sequence in antibodies derived from a particular species or belonging to a particular antibody class or subclass, while the remainder of the chain(s) is identical with or homologous to a corresponding sequence in antibodies derived from another species or belonging to another antibody class or subclass. Also included are fragments of such antibodies, so long as they exhibit the desired biological activity. See, U.S. Pat. No. 4,816,567; Morrison et at., Proc. Natl. Acad. Sci, 81: 6851-6855 (1985).

In another embodiment, a monoclonal antibody of the invention is a “humanized” antibody. Methods for humanizing non-human antibodies are well known in the art. See U.S. Pat. Nos. 5,585,089, and 5,693,762. Generally, a humanized antibody has one or more amino acid residues introduced into it from a source that is non-human. Humanization can be performed, for example, using methods described in the art (Jones et al., Nature 321: 522-525 (1986); Riechmann et al. Nature, 332: 323-327 (1988); Verhoeyen et al. Science 239: 1534-1536 (1988)), by substituting at least a portion of a rodent complementarity-determining region (CDR) for the corresponding regions of a human antibody.

Also encompassed by the invention are human antibodies that bind AFTI polypeptides. Using transgenic animals (e.g., mice) that are capable of producing a repertoire of human antibodies in the absence of endogenous immunoglobulin production such antibodies are produced by immunization with an AFTI antigen (i.e., having at least 6 contiguous amino acids), optionally conjugated to a carrier. See, for example, Jakobovits et al. Proc. Natl. Acad. Sci., 90: 2551-2555 (1993); Jakobovits et al., Nature 362: 255-258 (1993); Bruggermann et al. Year in Immune, 7: 33 (1993). Such technologies are available commercially, including the HuMab™ technology from Medarex, Inc. and the Xenomouse™ technology from Abgenix, Inc. In one method, such transgenic animals are produced by incapacitating the endogenous loci encoding the heavy and light immunoglobulin chains therein, and inserting loci encoding human heavy and light chain proteins into the genome thereof. Partially modified animals, that is those having less than the full complement of modifications, are then cross-bred to obtain an animal having all of the desired immune system modifications. When administered an immunogen, these transgenic animals produce antibodies with human (rather than e.g., murine) amino acid sequences, including variable regions which are immunospecific for these antigens. See PCT application nos. PCT/US96/05928 and PCT/US93/06926. Additional methods are described in U.S. Pat. No. 5,545,807, PCT application nos. PCT/US91/245, PCT/GB89/01207, and in EP 546 073 B1 and EP 546 073 A1. Human antibodies may also be produced by the expression of recombinant DNA in host cells or by expression in hybridoma cells as described herein. The human antibodies produced by the technologies described in this paragraph are collectively referred to as “fully human antibodies.”In an alternative embodiment, human antibodies can be produced from phage-display libraries (Hoogenboom et al., J. Mol. Biol. 227: 381 (1991); Marks et al. J. Mol. Biol. 222: 581 (1991). These processes mimic immune selection through the display of antibody repertoires on the surface of filamentous bacteriophage, and subsequent selection of phage by their binding to an antigen of choice. One such technique is described in PCT Application no. PCT/US98/17364, which describes the isolation of high affinity and functional agonistic antibodies for MPL- and msk-receptors using such an approach. The antibodies produced by the techniques described in this paragraph are referred to as “phage display antibodies.”

Chimeric, CDR grafted, and humanized antibodies are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

The anti-AFTI antibodies of the invention may be employed in any known assay method, such as competitive binding assays, direct and indirect sandwich assays, and immunoprecipitation assays (Sola, Monoclonal Antibodies: A Manual of Techniques, pp. 147-158 (CRC Press, Inc., 1987)) for the detection and quantitation of AFTI polypeptides. The antibodies will bind AFTI polypeptides with an affinity that is appropriate for the assay method being employed.

For diagnostic applications, in certain embodiments, anti-AFTI antibodies may be labeled with a detectable moiety. The detectable moiety can be any one that is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; or an enzyme, such as alkaline phosphatase, β-galactosidase, or horseradish peroxidase (Bayer et al., Meth. Enzymol., 184-138-163 (1990)).

Competitive binding assays rely on the ability of a labeled standard (e.g., an AFTI polypeptide, or an immunologically reactive portion thereof) to compete with the test sample analyte (an AFTI polypeptide) for binding with a limited amount of anti AFTI antibody. The amount of an AFTI polypeptide in the test sample is inversely proportional to the amount of standard that becomes bound to the antibodies. To facilitate determining the amount of standard that becomes bound, the antibodies typically are insolubilized before or after the competition, so that the standard and analyte that are bound to the antibodies may conveniently be separated from the standard and analyte that remain unbound.

Sandwich assays typically involve the use of two antibodies, each capable of binding to a different immunogenic portion, or epitope, of the protein to be detected and/or quantitated. In a sandwich assay, the test sample analyte is typically bound by a first antibody which is immobilized on a solid support, and thereafter a second antibody binds to the analyte, thus forming an insoluble three part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assays). For example, one type of sandwich assay is an enzyme-linked immunosorbent assay (ELISA), in which case the detectable moiety is an enzyme.

The selective binding agents, including anti-AFTI antibodies, also are useful for in vivo imaging. An antibody labeled with a detectable moiety may be administered to an animal, preferably into the bloodstream, and the presence and location of the labeled antibody in the host is assayed. The antibody may be labeled with any moiety that is detectable in an animal, whether by nuclear magnetic resonance, radiology, or other detection means known in the art.

Selective binding agents of the invention, including antibodies, may be used as therapeutics. These therapeutic agents are generally agonists or antagonists, in that they either enhance or reduce, respectively, at least one of the biological activities of an AFTI polypeptide. In one embodiment, antagonist antibodies of the invention are antibodies or binding fragments thereof that are capable of specifically binding to an AFTI polypeptide and that are capable of inhibiting or eliminating the functional activity of an AFTI polypeptide in vivo or in vitro. In preferred embodiments, the selective binding agent, e.g., an antagonist antibody, will inhibit the functional activity of an AFTI polypeptide by at least about 50%, and preferably by at least about 80%. In another embodiment, the selective binding agent may be an anti-AFTI polypeptide antibody that is capable of interacting with an AFTI binding partner (a ligand or receptor) thereby inhibiting or eliminating AFTI activity in vitro or in vivo. Selective binding agents, including agonist and antagonist anti-AFTI antibodies, are identified by screening assays which are well known in the art.

The invention also relates to a kit comprising AFTI selective binding agents (such as antibodies) and other reagents useful for detecting AFTI polypeptide levels in biological samples. Such reagents may include, a detectable label, blocking serum, positive and negative control samples, and detection reagents.

The AFTI polypeptides of the present invention can be used to clone AFTI receptors, using an expression cloning strategy. Radiolabeled (125-lodine) AFTI polypeptide or affinity/activity-tagged AFTI polypeptide (such as an Fc fusion or an alkaline phosphatase fusion) can be used in binding assays to identify a cell type or cell line or tissue that expresses AFTI receptor(s). RNA isolated from such cells or tissues can be converted to cDNA, cloned into a mammalian expression vector, and transfected into mammalian cells (such as COS or 293 cells) to create an expression library. A radiolabeled or tagged AFTI polypeptide can then be used as an affinity ligand to identify and isolate from this library the subset of cells that express the AFTI receptor(s) on their surface. DNA can then be isolated from these cells and transfected into mammalian cells to create a secondary expression library in which the fraction of cells expressing AFTI receptor(s) is many-fold higher than in the original library. This enrichment process can be repeated iteratively until a single recombinant clone containing an AFTI receptor is isolated. Isolation of the AFTI receptor(s) is useful for identifying or developing novel agonists and antagonists of the AFTI polypeptide signaling pathway. Such agonists and antagonists include soluble AFTI receptor(s), anti-AFTI receptor antibodies, small molecules, or antisense oligonucleotides, and they may be used for treating, preventing, or diagnosing one or more disease or disorder, including those described herein.

Assaying For Other Modulators Of AFTI Polypeptide Activity

In some situations, it may be desirable to identify molecules that are modulators, i.e., agonists or antagonists, of the activity of AFTI polypeptide. Natural or synthetic molecules that modulate AFTI polypeptide may be identified using one or more screening assays, such as those described herein. Such molecules may be administered either in an ex vivo manner, or in an in vivo manner by injection, or by oral delivery, implantation device, or the like. “Test molecule(s)” refers to the molecule(s) that is/are under evaluation for the ability to modulate (i.e., increase or decrease) the activity of an AFTI polypeptide. Most commonly, a test molecule will interact directly with an AFTI polypeptide. However, it is also contemplated that a test molecule may also modulate AFTI polypeptide activity indirectly, such as by affecting AFTI gene expression, or by binding to an AFTI binding partner (e.g., receptor or ligand). In one embodiment, a test molecule will bind to an AFTI polypeptide with an affinity constant of at least about 10⁻⁶ M, preferably about 10⁻⁸ M, more preferably about 10⁻⁹ M, and even more preferably about 10⁻¹⁰ M.

Methods for identifying compounds that interact with AFTI polypeptides are encompassed by the present invention. In certain embodiments, an AFTI polypeptide is incubated with a test molecule under conditions that permit the interaction of the test molecule with an AFTI polypeptide, and the extent of the interaction can be measured. The test molecule(s) can be screened in a substantially purified form or in a crude mixture.

In certain embodiments, an AFTI polypeptide agonist or antagonist may be a protein, peptide, carbohydrate, lipid, or small molecular weight molecule that interacts with AFTI polypeptide to regulate its activity. Molecules that regulate AFTI polypeptide expression include nucleic acids which are complementary to nucleic acids encoding an AFTI polypeptide, or are complementary to nucleic acids sequences which direct or control the expression of AFTI polypeptide, and which act as anti-sense regulators of expression.

Once a set of test molecules has been identified as interacting with an AFTI polypeptide, the molecules may be further evaluated for their ability to increase or decrease AFTI polypeptide activity. The measurement of the interaction of test molecules with AFTI polypeptides may be carried out in several formats, including cell-based binding assays, membrane binding assays, solution-phase assays and immunoassays. In general, test molecules are incubated with an AFTI polypeptide for a specified period of time, and AFTI polypeptide activity is determined by one or more assays for measuring biological activity.

The interaction of test molecules with AFTI polypeptides may also be assayed directly using polyclonal or monoclonal antibodies in an immunoassay. Alternatively, modified forms of AFTI polypeptides containing epitope tags as described herein may be used in immunoassays.

In the event that AFTi polypeptides display biological activity through an interaction with a binding partner (e.g., a receptor or a ligand), a variety of in vitro assays may be used to measure the binding of an AFTI polypeptide to the corresponding binding partner (such as a selective binding agent, receptor, or ligand). These assays may be used to screen test molecules for their ability to increase or decrease the rate and/or the extent of binding of an AFTI polypeptide to its binding partner. In one assay, an AFTI polypeptide is immobilized in the wells of a microtiter plate. Radiolabeled AFTI binding partner (for example, iodinated AFTI binding partner) and the test molecule(s) can then be added either one at a time (in either order) or simultaneously to the wells. After incubation, the wells can be washed and counted, using a scintillation counter, for radioactivity to determine the extent to which the binding partner bound to AFTI polypeptide. Typically, the molecules will be tested over a range of concentrations, and a series of control wells lacking one or more elements of the test assays can be used for accuracy in the evaluation of the results. An alternative to this method involves reversing the “positions” of the proteins, i.e., immobilizing AFTI binding partner to the microtiter plate wells, incubating with the test molecule and radiolabeled AFTI polypeptide, and determining the extent of AFTI polypeptide binding. See, for example, chapter 18, Current Protocols in Molecular Biology, Ausubel et al., eds., John Wiley & Sons, New York, N.Y. (1995).

As an alternative to radiolabelling, an AFTI polypeptide or its binding partner may be conjugated to biotin and the presence of biotinylated protein can then be detected using streptavidin linked to an enzyme, such as horseradish peroxidase (HRP) or alkaline phosphatase (AP), that can be detected colorometrically, or by fluorescent tagging of streptavidin. An antibody directed to an AFTI polypeptide or to an AFTI binding partner and conjugated to biotin may also be used and can be detected after incubation with enzyme-linked streptavidin linked to AP or HRP.

An AFTI polypeptide or an AFTI binding partner can also be immobilized by attachment to agarose beads, acrylic beads or other types of such inert solid phase substrates. The substrate-protein complex can be placed in a solution containing the complementary protein and the test compound. After incubation, the beads can be precipitated by centrifugation, and the amount of binding between an AFTI polypeptide and its binding partner can be assessed using the methods described herein. Alternatively, the substrate-protein complex can be immobilized in a column, and the test molecule and complementary protein are passed through the column. The formation of a complex between an AFTI polypeptide and its binding partner can then be assessed using any of the techniques set forth herein, i.e., radiolabelling, antibody binding, or the like.

Another in vitro assay that is useful for identifying a test molecule that increases or decreases the formation of a complex between an AFTI binding protein and an AFTI binding partner is a surface plasmon resonance detector system such as the BIAcore assay system (Pharmacia, Piscataway, N.J.). The BIAcore system may be carried out using the manufacturer's protocol. This assay essentially involves the covalent binding of either AFTI polypeptide or an AFTI binding partner to a dextran-coated sensor chip which is located in a detector. The test compound and the other complementary protein can then be injected, either simultaneously or sequentially, into the chamber containing the sensor chip. The amount of complementary protein that binds can be assessed based on the change in molecular mass that is physically associated with the dextran-coated side of the sensor chip; the change in molecular mass can be measured by the detector system.

In some cases, it may be desirable to evaluate two or more test compounds together for their ability to increase or decrease the formation of a complex between an AFTI polypeptide and an AFTI binding partner. In these cases, the assays set forth herein can be readily modified by adding such additional test compound(s) either simultaneous with, or subsequent to, the first test compound. The remainder of the steps in the assay are as set forth herein.

In vitro assays such as those described herein may be used advantageously to screen large numbers of compounds for effects on complex formation by AFTI polypeptide and AFTI binding partner. The assays may be automated to screen compounds generated in phage display, synthetic peptide, and chemical synthesis libraries.

Compounds that increase or decrease the formation of a complex between an AFTI polypeptide and an AFTI binding partner may also be screened in cell culture using cells and cell lines expressing either AFTI polypeptide or AFTI binding partner. Cells and cell lines may be obtained from any mammal, but preferably will be from polypeptidehuman or other primate, canine, or rodent sources. The binding of an AFTI polypeptide to cells expressing AFTI binding partner at the surface is evaluated in the presence or absence of test molecules, and the extent of binding may be determined by, for example, flow cytometry using a biotinylated antibody to an AFTI binding partner. Cell culture assays can be used advantageously to further evaluate compounds that score positive in protein binding assays described herein.

Cell cultures can also be used to screen the impact of a drug candidate. For example, drug candidates may decrease or increase the expression of the AFTI gene. In certain embodiments, the amount of AFTI polypeptide that is produced may be measured after exposure of the cell culture to the drug candidate. In certain embodiments, one may detect the actual impact of the drug candidate on the cell culture. For example, the overexpression of a particular gene may have a particular impact on the cell culture. In such cases, one may test a drug candidate's ability to increase or decrease the expression of the gene or its ability to prevent or inhibit a particular impact on the cell culture. In other examples, the production of a particular metabolic product such as a fragment of a polypeptide, may result in, or be associated with, a disease or pathological condition. In such cases, one may test a drug candidate's ability to decrease the production of such a metabolic product in a cell culture.

Internalizing Proteins

The tat protein sequence (from HIV) can be used to internalize proteins into a cell. See e.g., Falwell et al., Proc. Natl. Acad. Sci., 91: 664-668 (1994). For example, an 11 amino acid sequence (YGRKKRRQRRR) (SEQ ID NO:5) of the HIV tat protein (termed the “protein transduction domain”, or TAT PDT) has been described as mediating delivery across the cytoplasmic membrane and the nuclear membrane of a cell. See Schwarze et al., Science, 285: 1569-1572 (1999); and Nagahara et al., Nature Medicine, 4: 1449-1452 (1998). In these procedures, FITC-constructs (FITC-GGGGYGRKKRRQRRR) (SEQ ID NO:6) are prepared which bind to cells as observed by fluorescence-activated cell sorting (FACS) analysis, and these constructs penetrate tissues after i.p. adminstration. Next, tat-bgal fusion proteins are constructed. Cells treated with this construct demonstrated b-gal activity. Following injection, a number of tissues, including liver, kidney, lung, heart, and brain tissue have been found to demonstrate expression using these procedures. It is believed that these constructions underwent some degree of unfolding in order to enter the cell; as such, refolding may be required after entering the cell.

It will thus be appreciated that the tat protein sequence may be used to internalize a desired protein or polypeptide into a cell. For example, using the tat protein sequence, an AFTI antagonist (such as an anti-AFTI selective binding agent, small molecule, soluble receptor, or antisense oligonucleotide) can be administered intracellularly to inhibit the activity of an AFTI molecule. As used herein, the term “AFTI molecule” refers to both AFTI nucleic acid molecules and AFTI polypeptides as defined herein. Where desired, the AFTI protein itself may also be internally administered to a cell using these procedures. See also, Strauss, E., “Introducing Proteins Into the Body's Cells”, Science, 285: 1466-1467 (1999).

Cell Source Identification Using AFTI Polypeptides

In accordance with certain embodiments of the invention, it may be useful to be able to determine the source of a certain cell type associated with an AFTI polypeptide. For example, it may be useful to determine the origin of a disease or pathological condition as an aid in selecting an appropriate therapy. In certain embodiments, nucleic acids encoding an AFTI polypeptide can be used as a probe to identify cells that express or contain a polynucleotide encoding an AFTI polypeptide by screening the nucleic acids of the cells with such a probe. In other embodiments, one may use anti-AFTI polypeptide antibodies to test for the presence of AFTI polypeptide in cells, and thus, determine if such cells express an AFTI polypeptide or if they are derived from a cell known to express an AFTI polypeptide.

Therapeutic Uses

A non-exclusive list of acute and chronic diseases that can be treated, diagnosed, ameliorated, or prevented with the polypeptides and nucleic acids of the invention include those diseases treatable by inhibition of IL-1 and/or TNF activity. These diseases are summarized below.

IL-1 Inhibition

One of the most potent inflammatory cytokines yet discovered is interleukin-1 (IL-1). IL-1 is thought to be a key mediator in many diseases and medical conditions. It is manufactured (though not exclusively) by cells of the macrophage/monocyte lineage and may be produced in two forms: IL-1 alpha (IL-1α) and IL-1 beta (IL-1β).

A disease or medical condition is considered to be an “interleukin-1 mediated disease” if the spontaneous or experimental disease or medical condition is associated with elevated levels of IL-1 in bodily fluids or tissue or if cells or tissues taken from the body produce elevated levels of IL-1 in culture. In many cases, such interleukin-1 mediated diseases are also recognized by the following additional two conditions: (1) pathological findings associated with the disease or medical condition can be mimicked experimentally in animals by administration of IL-1 or upregulation of expression of IL-1; and (2) a pathology induced in experimental animal models of the disease or medical condition can be inhibited or abolished by treatment with agents that inhibit the action of IL-1. In most interleukin-1 mediated diseases at least two of the three conditions are met, and in many interleukin-1 mediated diseases all three conditions are met.

A non-exclusive list of acute and chronic interleukin-1 (IL-i)-mediated diseases includes, but is not limited to, the following:

acute pancreatitis;

ALS;

Alzheimer's disease;

cachexia/anorexia, including AIDS-induced cachexia;

asthma and other pulmonary diseases;

atherosclerosis;

autoimmune vasculitis;

chronic fatigue syndrome;

Clostridium associated illnesses, including Clostridium-associated diarrhea;

coronary conditions and indications, including congestive heart failure,

coronary restenosis,

myocardial infarction,

myocardial dysfunction (e.g., related to sepsis), and coronary artery bypass graft;

cancer, such as multiple myeloma and myelogenous (e.g., AML and CML) and other leukemias, as well as tumor metastasis;

diabetes (e.g., insulin diabetes);

endometriosis;

fever;

fibromyalgia;

glomerulonephritis;

graft versus host disease/transplant rejection;

hemohorragic shock;

hyperalgesia;

inflammatory bowel disease;

inflammatory conditions of a joint, including osteoarthritis, psoriatic arthritis and rheumatoid arthritis (RA);

inflammatory eye disease, as may be associated with, for example, corneal transplant;

polypeptideischemia, including cerebral ischemia (e.g., brain injury as a result of trauma, epilepsy, hemorrhage or stroke, each of which may lead to neurodegeneration);

Kawasaki's disease;

learning impairment;

lung diseases (e.g., ARDS);

multiple sclerosis;

myopathies (e.g., muscle protein metabolism, esp. in sepsis);

neurotoxicity (e.g., as induced by HIV);

osteoporosis; pain, including cancer-related pain;

Parkinson's disease;

periodontal disease;

pre-term labor;

psoriasis;

reperfusion injury;

septic shock;

side effects from radiation therapy;

temporal mandibular joint disease;

sleep disturbance;

uveitis;

or an inflammatory condition resulting from strain, sprain, cartilage damage, trauma, orthopedic surgery, infection or other disease processes.

TNF-α Inhibition

Many diseases and medical conditions are mediated by TNF and are usually categorized as inflammatory conditions. A “TNF-mediated disease” is a spontaneous or -experimental disease or medical condition associated with elevated levels of TNF in bodily fluids. In many cases, such TNF-mediated diseases may also be characterized by the following: (1) pathological findings associated with the disease or medical condition can be mimicked experimentally in animals by the administration or upregulation of expression of TNF; (2) when pathology induced in experimental animal models, the disease or medical condition can be inhibited or abolished by treatment with agents that inhibit the action of TNF.

A non-exclusive list of acute and chronic TNF-mediated diseases includes, but is not limited to, the following:

cachexia/anorexia;

cancer (e.g., leukemias);

chronic fatigue syndrome;

coronary conditions and indications, including congestive heart failure, coronary restenosis, myocardial infarction, myocardial dysfunction (e.g., related to sepsis), and coronary artery bypass graft;

depression;

diabetes, including juvenile onset Type 1,

diabetes mellitus, and insulin resistance (e.g., as associated with obesity);

endometriosis,

endometritis, and related conditions;

fibromyalgia or analgesia;

graft versus host rejection;

hyperalgesia;

inflammatory bowel diseases, including Crohn's disease and Clostridium difficile-associated diarrhea;

ischemia, including cerebral ischemia (brain injury as a result of trauma,

epilepsy,

hemorrhage or stroke, each of which may lead to neurodegeneration);

lung diseases (e.g., adult respiratory distress syndrome, asthma, and pulmonary fibrosis);

multiple sclerosis;

neuroinflammatory diseases;

ocular diseases and conditions, including corneal transplant, ocular degeneration and uveitis;

pain, including cancer-related pain;

pancreatitis;

periodontal diseases;

Pityriasis rubra pilaris (PRP);

prostatitis (bacterial or non-bacterial) and related conditions;

psoriasis and related conditions;

pulmonary fibrosis;

reperfusion injury;

rheumatic diseases, including rheumatoid arthritis, osteoarthritis, juvenile (rheumatoid) arthritis, seronegative polyarthritis, ankylosing spondylitis, Reiter's syndrome and reactive arthritis, Still's disease, psoriatic arthritis, enteropathic arthritis, polymyositis, dermatomyositis, scleroderma, systemic sclerosis, vasculitis (e.g., Kawasaki's disease), cerebral vasculitis, Lyme disease, staphylococcal-induced (“septic”) arthritis, Sjogren's syndrome, rheumatic fever, polychondritis and polymyalgia rheumatica and giant cell arteritis);

septic shock;

side effects from radiation therapy;

systemic lupus erythematosus (SLE);

temporal mandibular joint disease;

thyroiditis;

tissue transplantation or an inflammatory condition resulting from strain, sprain, cartilage damage, trauma, orthopedic surgery, infection (e.g., HIV, Clostridium difficile and related species) or other disease process.

An inhibitor of an AFTI (e.g., anti-AFTI antibodies) may be useful in therapeutic applications wherein a patient would benefit from upregulation of TNF and/or IL-1. Such diseases and applications include, but are not limited to, cancer, transplantation-graft-versus-host disease.

AFTI Compositions and Administration

Therapeutic compositions are within the scope of the present invention. Such AFTI pharmaceutical compositions may comprise a therapeutically effective amount of an AFTI polypeptide or an AFTI nucleic acid molecule in admixture with a pharmaceutically or physiologically acceptable formulation agent selected for suitability with the mode of administration. Pharmaceutical compositions may comprise a therapeutically effective amount of one or more AFTI selective binding agents in admixture with a pharmaceutically or physiologically acceptable formulation agent selected for suitability with the mode of administration.

Acceptable formulation materials preferably are nontoxic to recipients at the dosages and concentrations employed.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine), antimicrobials, antioxidants (such as ascorbic acid, sodium sulfite or sodium hydrogen-sulfite), buffers (such as borate, bicarbonate, Tris-HCl, citrates, phosphates, other organic acids), bulking agents (such as mannitol or glycine), chelating agents (such as ethylenediamine tetraacetic acid (EDTA)), complexing agents (such as caffeine, polyvinylpyrrolidone, beta-cyclodextrin or hydroxypropyl-beta-cyclodextrin), fillers, monosaccharides, disaccharides, and other carbohydrates (such as glucose, mannose, or dextrins), proteins (such as serum albumin, gelatin or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid or hydrogen peroxide), solvents (such as glycerin, propylene glycol or polyethylene glycol), sugar alcohols (such as mannitol or sorbitol), suspending agents, surfactants or wetting agents (such as pluronics, PEG, sorbitan esters, polysorbates such as polysorbate 20, polysorbate 80, triton, tromethamine, lecithin, cholesterol, tyloxapal), stability enhancing agents (sucrose or sorbitol), tonicity enhancing agents (such as alkali metal halides (preferably sodium or potassium chloride), mannitol sorbitol), delivery vehicles, diluents, excipients and/or pharmaceutical adjuvants. (Remington's Pharmaceutical Sciences, 18th Edition, A. R. Gennaro, ed., Mack Publishing Company [1990]).

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the AFTI molecule.

The primary vehicle or carrier in a pharmaceutical composition may be either aqueous or non-aqueous in nature. For example, a suitable vehicle or carrier may be water for injection, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefor. In one embodiment of the present invention, AFTI polypeptide compositions may be prepared for storage by mixing the selected composition having the desired degree of purity with optional formulation agents (Remington's Pharmaceutical Sciences, supra) in the form of a lyophilized cake or an aqueous solution. Further, the AFTI polypeptide product may be formulated as a lyophilizate using appropriate excipients such as sucrose.

The AFTI pharmaceutical compositions can be selected for parenteral delivery. Alternatively, the compositions may be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at slightly lower pH, typically within a pH range of from about 5 to about 8.

When parenteral administration is contemplated, the therapeutic compositions for puse in this invention may be in the form of a pyrogen-free, parenterally acceptable aqueous solution comprising the desired AFTI molecule in a pharmaceutically acceptable vehicle. A particularly suitable vehicle for parenteral injection is sterile distilled water in which a AFTI molecule is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), or beads, or liposomes, that provides for the controlled or sustained release of the product which may then be delivered as a depot injection. Hyaluronic acid may also be used, and this may have the effect of promoting sustained duration in the circulation. Other suitable means for the introduction of the desired molecule include implantable drug delivery devices.

In one embodiment, a pharmaceutical composition may be formulated for inhalation. For example, an AFTI molecule may be formulated as a dry powder for inhalation. AFTI polypeptide or AFTI nucleic acid molecule inhalation solutions may also be formulated with a propellant for aerosol delivery. In yet another embodiment, solutions may be nebulized. Pulmonary administration is further described in PCT application no. PCT/US94/001875, which describes pulmonary delivery of chemically modified proteins.

It is also contemplated that certain formulations may be administered orally. In one embodiment of the present invention, AFTI molecules that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the AFTI molecule. Diluents, flavorings, low melting point waxes, vegetable oils, lubricants, suspending agents, tablet disintegrating agents, and binders may also be employed.

Another pharmaceutical composition may involve an effective quantity of AFTI molecules in a mixture with non-toxic excipients which are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional AFTI pharmaceutical compositions will be evident to those skilled in the art, including formulations involving AFTI polypeptides in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT/US93/00829 which describes controlled release of porous polymeric microparticles for the delivery of pharmaceutical compositions. Additional examples of sustained-release preparations include semipermeabie polymer matrices in the form of shaped articles, e.g. films, or microcapsules. Sustained release matrices may include polyesters, hydrogels, polylactides (U.S. Pat. No. 3,773,919, EP 58,481), copolymers of L-glutamic acid and gamma ethyl-L-glutamate (Sidman et al., Biopolymers, 22: 547-556 (1983)), poly (2-hydroxyethyl-methacrylate) (Langer et al., J. Biomed. Mater. Res., 15: 167-277 (1981) and Langer, Chem. Tech., 12: 98-105 (1982)), ethylene vinyl acetate (Langer et al., supra) or poly-D(−)-3-hydroxybutyric acid (EP 133,988). Sustained-release compositions also may include liposomes, which can be prepared by any of several methods known in the art. See e.g., Eppstein et al., Proc. Natl. Acad. Sci. USA, 82: 3688-3692 (1985); EP 36,676; EP 88,046; EP 143,949. The AFTI pharmaceutical composition to be used for in vivo administration typically must be sterile. This may be accomplished by filtration through sterile filtration membranes. Where the composition is lyophilized, sterilization using these methods may be conducted either prior to, or following, lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single- dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

An effective amount of an AFTI pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the AFTI molecule is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 μg/kg to up to about 100 mg/kg or more, depending on the factors mentioned above. In other embodiments, the dosage may range from 0.1 μg/kg up to about 100 mg/kg; or 1 μg/kg up to about 100 mg/kg; or 5 μg/kg up to about 100 mg/kg.

The frequency of dosing will depend upon the pharmacokinetic parameters of the AFTI molecule in the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via implantation device or catheter. Further refinement of the appropriate dosage is routinely made by those of ordinary skill in the art and is within the ambit of tasks routinely performed by them. Appropriate dosages may be ascertained through use of appropriate dose-response data.

The route of administration of the pharmaceutical composition is in accord with known methods, e.g. oral, injection by intravenous, intraperitoneal, intracerebral (intra-parenchymal), intracerebroventricular, intramuscular, intra-ocular, intraarterial, intraportal, or intralesional routes, or by sustained release systems or implantation device. Where desired, the compositions may be administered by bolus injection or continuously by infusion, or by implantation device.

Alternatively or additionally, the composition may be administered locally via implantation of a membrane, sponge, or other appropriate material on to which the desired molecule has been absorbed or encapsulated. Where an implantation device is used, the device may be implanted into any suitable tissue or organ, and delivery of the desired molecule may be via diffusion, timed release bolus, or continuous administration.

In some cases, it may be desirable to use AFTI pharmaceutical compositions in an ex vivo manner. In such instances, cells, tissues, or organs that have been removed from the patient are exposed to AFTI pharmaceutical compositions after which the cells, tissues and/or organs are implanted back into the patient.

In other cases, an AFTI polypeptide can be delivered by implanting certain cells that have been genetically engineered, using methods such as those described herein, to express and secrete the polypeptide. Such cells may be animal or human cells, and may be autoiogous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

It may be desirable to treat isolated cell populations (such as T-cells) with one or more AFTI polypeptides. This can be accomplished by exposing the isolated cells to the polypeptide directly, where it is in a form that is permeable to the cell membrane.

Additional embodiments of the present invention relate to cells and methods (e.g., homologous recombination and/or other recombinant production methods) for both the in vitro production of therapeutic polypeptides and for the production and delivery of therapeutic polypeptides by gene therapy or cell therapy. Homologous and other recombination methods may be used to modify a cell that contains a normally transcriptionally silent AFTI gene, or an under expressed gene, and thereby produce a cell that expresses therapeutically efficacious amounts of AFTI polypeptides.

Homologous recombination is a technique originally developed for targeting genes to induce or correct mutations in transcriptionally active genes (Kucherlapati, Prog. in Nucl. Acid Res. & Mol. Biol., 36: 301, 1989). The basic technique was developed as a method for introducing specific mutations into specific regions of the mammalian genome (Thomas et al., Cell, 44: 419-428, 1986; Thomas and Capecchi, Cell, 51: 503-512, 1987; Doetschman et al., Proc. Natl Acad. Sci., 85: 8583-8587, 1988) or to correct specific mutations within defective genes (Doetschman et al., Nature, 330: 576-578, 1987). Exemplary homologous recombination techniques are described in U.S. Pat. No. 5,272,071 (EP 9193051, EP Publication No. 505500; PCT/US90/07642, International Publication No. WO 91/09955).

Through homologous recombination, the DNA sequence to be inserted into the genome can be directed to a specific region of the gene of interest by attaching it to targeting DNA. The targeting DNA is a nucleotide sequence that is complementary (homologous) to a region of the genomic DNA. Small pieces of targeting DNA that are complementary to a specific region of the genome are put in contact with the parental strand during the DNA replication process. It is a general property of DNA that has been inserted into a cell to hybridize, and therefore, recombine with other pieces of endogenous DNA through shared homologous regions. If this complementary strand is attached to an oligonucleotide that contains a mutation or a different sequence or an additional nucleotide, it too is incorporated into the newly synthesized strand as a result of the recombination. As a result of the proofreading function, it is possible for the new sequence of DNA to serve as the template. Thus, the transferred DNA is incorporated into the genome.

Attached to these pieces of targeting DNA are regions of DNA that may interact with or control the expression of a AFTI polypeptide, e.g., flanking sequences. For example, a promoter/enhancer element, a suppresses or an exogenous transcription modulatory element is inserted in the genome of the intended host cell in proximity and orientation sufficient to influence the transcription of DNA encoding the desired AFTI polypeptide. The control element controls a portion of the DNA present in the host cell genome. Thus, the expression of the desired AFTI polypeptide may be achieved not by transfection of DNA that encodes the AFTI gene itself, but rather by the use of targeting DNA (containing regions of homology with the endogenous gene of interest) coupled with DNA regulatory segments that provide the endogenous gene sequence with recognizable signals for transcription of an AFTI polypeptide.

In an exemplary method, the expression of a desired targeted gene in a cell (i.e., a desired endogenous cellular gene) is altered via homologous recombination into the cellular genome at a preselected site, by the introduction of DNA that includes at least a regulatory sequence, an exon and a splice donor site. These components are introduced into the chromosomal (genomic) DNA in such a manner that this, in effect, results in the production of a new transcription unit (in which the regulatory sequence, the exon and the splice donor site present in the DNA construct are operatively linked to the endogenous gene). As a result of the introduction of these components into the chromosomal DNA, the expression of the desired endogenous gene is altered.

Altered gene expression, as described herein, encompasses activating (or causing to be expressed) a gene that is normally silent (unexpressed) in the cell as obtained, as well as increasing the expression of a gene that is not expressed at physiologically significant levels in the cell as obtained. The embodiments further encompass changing the pattern of regulation or induction such that it is different from the pattern of regulation or induction that occurs in the cell as obtained, and reducing (including eliminating) the expression of a gene that is expressed in the cell as obtained.

One method by which homologous recombination can be used to increase, or cause, AFTI polypeptide production from a cell's endogenous AFTI gene involves first using homologous recombination to place a recombination sequence from a site-specific recombination system (e.g., Cre/loxP, FLP/FRT) (Sauer, Current Opinion In Biotechnology, 5: 521-527, 1994; Sauer, Methods In Enzymology, 225: 890-900, 1993) upstream (that is, 5′ to) of the cell's endogenous genomic AFTI polypeptide coding region. A plasmid containing a recombination site homologous to the site that was placed just upstream of the genomic AFTI polypeptide coding region is introduced into the modified cell line along with the appropriate recombinase enzyme. This recombinase causes the plasmid to integrate, via the plasmid's recombination site, into the recombination site located just upstream of the genomic AFTI polypeptide coding region in the cell line (Baubonis and Sauer, Nucleic Acids Res., 21: 2025-2029, 1993; O'Gorman et al., Science, 251: 1351-1355, 1991). Any flanking sequences known to increase transcription (e.g., enhancer/promoter, intron, translational enhancer), if properly positioned in this plasmid, would integrate in such a manner as to create a new or modified transcriptional unit resulting in de novo or increased AFTI polypeptide production from the cell's endogenous AFTI gene.

A further method to use the cell line in which the site specific recombination sequence had been placed just upstream of the cell's endogenous genomic AFTI polypeptide coding region is to use homologous recombination to introduce a second recombination site elsewhere in the cell line's genome. The appropriate recombinase enzyme is then introduced into the two-recombination-site cell line, causing a recombination event (deletion, inversion, translocation) (Sauer, Current Opinion In Biotechnology, supra, 1994; Sauer, Methods In Enzymology, supra, 1993) that would create a new or modified transcriptional unit resulting in de novo or increased AFTI polypeptide production from the cell's endogenous AFTI gene.

An additional approach for increasing, or causing, the expression of AFTI polypeptide from a cell's endogenous AFTI gene involves increasing, or causing, the expression of a gene or genes (e.g., transcription factors) and/or decreasing the expression of a gene or genes (e.g., transcriptional repressors) in a manner that results in de novo or increased AFTI polypeptide production from the cell's endogenous AFTI gene. This method includes the introduction of a non-naturally occurring polypeptide (e.g., a polypeptide comprising a site specific DNA binding domain fused to a transcriptional factor domain) into the cell such that de novo or increased AFTI polypeptide production from the cell's endogenous AFTI gene results.

The present invention further relates to DNA constructs useful in the method of altering expression of a target gene. In certain embodiments, the exemplary DNA constructs comprise: (a) one or more targeting sequences; (b) a regulatory sequence; (c) an exon; and (d) an unpaired splice-donor site. The targeting sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is homologous to the preselected site in the cellular chromosomal DNA with which homologous recombination is to occur. In the construct, the exon is generally 3′ of the regulatory sequence and the splice-donor site is 3′ of the exon.

If the sequence of a particular gene is known, such as the nucleic acid sequence of AFTI polypeptide presented herein, a piece of DNA that is complementary to a selected region of the gene can be synthesized or otherwise obtained, such as by appropriate restriction of the native DNA at specific recognition sites bounding the region of interest. This piece serves as a targeting sequence(s) upon insertion into the cell and will hybridize to its homologous region within the genome. If this hybridization occurs during DNA replication, this piece of DNA, and any additional sequence attached thereto, will act as an Okazaki fragment and will be incorporated into the newly synthesized daughter strand of DNA. The present invention, therefore, includes nucleotides encoding a AFTI polypeptide, which nucleotides may be used as targeting sequences.

AFTI polypeptide cell therapy, e.g., the implantation of cells producing AFTI polypeptides, is also contemplated. This embodiment involves implanting cells capable of synthesizing and secreting a biologically active form of AFTI polypeptide. Such AFTI polypeptide-producing cells can be cells that are natural producers of AFTI polypeptides or may be recombinant cells whose ability to produce AFTI polypeptides has been augmented by transformation with a gene encoding the desired AFTI polypeptide or with a gene augmenting the expression of AFTI polypeptide. Such a modification may be accomplished by means of a vector suitable for delivering the gene as well as promoting its expression and secretion. In order to minimize a potential immunological reaction in patients being administered an AFTI polypeptide, as may occur with the administration of a polypeptide of a foreign species, it is preferred that the natural cells producing AFTI polypeptide be of human origin and produce human AFTI polypeptide. Likewise, it is preferred that the recombinant cells producing AFTI polypeptide be transformed with an expression vector containing a gene encoding a human AFTI polypeptide.

Implanted cells may be encapsulated to avoid the infiltration of surrounding tissue. Human or non-human animal cells may be implanted in patients in biocompatible, semipermeable polymeric enclosures or membranes that allow the release of AFTI polypeptide, but that prevent the destruction of the cells by the patient's immune system or by other detrimental factors from the surrounding tissue. Alternatively, the patient's own cells, transformed to produce AFTI polypeptides ex vivo, may be implanted directly into the patient without such encapsulation. Techniques for the encapsulation of living cells are known in the art, and the preparation of the encapsulated cells and their implantation in patients may be routinely accomplished. For example, Baetge et al. (WO95/05452; PCT/US94/09299) describe membrane capsules containing genetically engineered cells for the effective delivery of biologically active molecules. The capsules are biocompatible and are easily retrievable. The capsules encapsulate cells transfected with recombinant DNA molecules comprising DNA sequences coding for biologically active molecules operatively linked to promoters that are not subject to down regulation in vivo upon implantation into a mammalian host. The devices provide for the delivery of the molecules from living cells to specific sites within a recipient. In addition, see U.S. Pat. Nos. 4,892,538, 5,011,472, and 5,106,627. A system for encapsulating living cells is described in PCT Application no. PCT/US91/00157 of Aebischer et al. See also, PCT Application no. PCT/US91/00155 of Aebischer et al., Winn et al., Exper. Neurol., 113: 322-329 (1991), Aebischer et al, Exper. Neurol., 111: 269-275 (1991); and Tresco et al., ASAIO, 38: 17-23 (1992).

In vivo and in vitro gene therapy delivery of AFTI polypeptides is also envisioned. One example of a gene therapy technique is to use the AFTI gene (either genomic DNA, cDNA, and/or synthetic DNA) encoding a AFTI polypeptide which may be operably linked to a constitutive or inducible promoter to form a “gene therapy DNA construct”. The promoter may be homologous or heterologous to the endogenous AFTI gene, provided that it is active in the cell or tissue type into which the construct will be inserted. Other components of the gene therapy DNA construct may optionally include, DNA molecules designed for site-specific integration (e.g., endogenous sequences useful for homologous recombination), tissue-specific promoter, enhancer(s) or silencer(s), DNA molecules capable of providing a selective advantage over the parent cell, DNA molecules useful as labels to identify transformed cells, negative selection systems, cell specific binding agents (as, for example, for cell targeting), cell-specific internalization factors, and transcription factors to enhance expression by a vector as well as factors to enable vector manufacture.

A gene therapy DNA construct can then be introduced into cells (either ex vivo or in vivo) using viral or non-viral vectors. One means for introducing the gene therapy DNA construct is by means of viral vectors as described herein. Certain vectors, such as retroviral vectors, will deliver the DNA construct to the chromosomal DNA of the cells, and the gene can integrate into the chromosomal DNA. Other vectors will function as episomes, and the gene therapy DNA construct will remain in the cytoplasm.

In yet other embodiments, regulatory elements can be included for the controlled expression of the AFTI gene in the target cell. Such elements are turned on in response to an appropriate effector. In this way, a therapeutic polypeptide can be expressed when desired. One conventional control means involves the use of small molecule dimerizers or rapalogs (as described in WO9641865 (PCT/US96/099486); WO9731898 (PCT/US97/03137) and WO9731899 (PCT/US95/03157)) used to dimerize chimeric proteins that contain a small molecule-binding domain and a domain capable of initiating biological process, such as a DNA-binding protein or transcriptional activation protein. The dimerization of the proteins can be used to initiate transcription of the AFTi gene.

Other suitable control systems or gene switches include, but are not limited to, the following systems. Mifepristone (RU486) is used as a progesterone antagonist. The binding of a modified progesterone receptor ligand-binding domain to the progesterone antagonist activates transcription by forming a dimer of two transcription factors which then pass into the nucleus to bind DNA. The ligand binding domain is modified to eliminate the ability of the receptor to bind to the natural ligand. The modified steroid hormone receptor system is further described in U.S. Pat. No. 5,364,791; WO964091 1, and WO9710337.

Yet another control system uses ecdysone (a fruit fly steroid hormone) which binds to and activates an ecdysone receptor (cytoplasmic receptor). The receptor then translocates to the nucleus to bind a specific DNA response element (promoter from ecdysone-responsive gene). The ecdysone receptor includes a transactivation domain/DNA-binding domain/ligand-binding domain to initiate transcription. The ecdysone system is further described in U.S. Pat. No. 5,514,578; WO9738117; WO9637609; and WO9303162.

Another control system uses a positive tetracycline-controllable transactivator. This system involves a mutated tet repressor protein DNA-binding domain (mutated tet R-4 amino acid changes which resulted in a reverse tetracycline-regulated transactivator protein, i.e., it binds to a tet operator in the presence of tetracycline) linked to a polypeptide that activates transcription. Such systems are described in U.S. Pat. Nos. 5,464,758; 5,650,298 and 5,654,168. Docket No.: 06843.0035

Additional expression control systems and nucleic acid constructs are described in U.S. Pat. Nos. 5,741,679 and 5,834,186, to Innovir Laboratories Inc.

In vivo gene therapy may be accomplished by introducing the gene encoding an AFTI polypeptide into cells via local injection of an AFTI nucleic acid molecule or by other appropriate viral or non-viral delivery vectors. Hefti, Neurobiology, 25: 1418-1435 (1994). For example, a nucleic acid molecule encoding an AFTI polypeptide may be contained in an adeno-associated virus (AAV) vector for delivery to the targeted cells (e.g., Johnson, International Publication No. WO95/34670; International Application No. PCT/US95/07178). The recombinant AAV genome typically contains AAV inverted terminal repeats flanking a DNA sequence encoding an AFTI polypeptide operably linked to functional promoter and polyadenylation sequences.

Alternative suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells that have been treated in vitro to insert a DNA segment encoding a therapeutic protein. Additional methods and materials for the practice of gene therapy techniques are described in U.S. Pat. No. 5,631,236 involving adenoviral vectors; U.S. Pat. No. 5,672,510 involving retroviral vectors; and U.S. Pat. No. 5,635,399 involving retroviral vectors expressing cytokines.

Nonviral delivery methods include, but are not limited to, liposome-mediated transfer, naked DNA delivery (direct injection), receptor-mediated transfer (ligand-DNA complex), electroporation, calcium phosphate precipitation, and microparticle bombardment (e.g., gene gun). Gene therapy materials and methods may also include the use of inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, and transcription factors to enhance expression by a vector as well as methods of vector manufacture. Such additional methods and materials for the practice of gene therapy techniques are described in U.S. Pat. No. 4,970,154 involving electroporation techniques; WO96/40958 involving nuclear ligands; U.S. Pat. No. 5,679,559 describing a lipoprotein-containing system for gene delivery; U.S. Pat. No. 5,676,954 involving liposome carriers; U.S. Pat. No. 5,593,875 concerning methods for calcium phosphate transfection; and U.S. Pat. No. 4,945,050 wherein biologically active particles are propelled at cells at a speed whereby the particles penetrate the surface of the cells and become incorporated into the interior of the cells.

It is also contemplated that AFTI gene therapy or cell therapy can further include the delivery of one or more additional polypeptide(s) in the same or a different cell(s). Such cells may be separately introduced into the patient, or the cells may be contained in a single implantable device, such as the encapsulating membrane described above, or the cells may be separately modified by means of viral vectors.

A way to increase endogenous AFTI polypeptide expression in a cell via gene therapy is to insert one or more enhancer elements into the AFTI polypeptide promoter, where the enhancer element(s) can serve to increase transcriptional activity of the AFTI gene. The enhancer element(s) used will be selected based on the tissue in which one desires to activate the gene(s); enhancer elements known to confer promoter activation in that tissue will be selected. For example, if a gene encoding a AFTI polypeptide is to be “turned on” in T-cells, the Ick promoter enhancer element may be used. Here, the functional portion of the transcriptional element to be added may be inserted into a fragment of DNA containing the AFTI polypeptide promoter (and optionally, inserted into a vector and/or 5′ and/or 3′ flanking sequence(s), etc.) using standard cloning techniques. This construct, known as a “homologous recombination construct”, can then be introduced into the desired cells either ex vivo or in vivo.

Gene therapy also can be used to decrease AFTI polypeptide expression by modifying the nucleotide sequence of the endogenous promoter(s). Such modification is typically accomplished via homologous recombination methods. For example, a DNA molecule containing all or a portion of the promoter of the AFTI gene(s) selected for inactivation can be engineered to remove and/or replace pieces of the promoter that regulate transcription. For example the TATA box and/or the binding site of a transcriptional activator of the promoter may be deleted using standard molecular biology techniques; such deletion can inhibit promoter activity thereby repressing the transcription of the corresponding AFTI gene. The deletion of the TATA box or the transcription activator binding site in the promoter may be accomplished by generating a DNA construct comprising all or the relevant portion of the AFTI polypeptide promoter(s) (from the same or a related species as the AFTI gene(s) to be regulated) in which one or more of the TATA box and/or transcriptional activator binding site nucleotides are mutated via substitution, deletion and/or insertion of one or more nucleotides. As a result, the TATA box and/or activator binding site has decreased activity or is rendered completely inactive. The construct will typically contain at least about 500 bases of DNA that correspond to the native (endogenous) 5′ and 3′ DNA sequences adjacent to the promoter segment that has been modified. The construct may be introduced into the appropriate cells (either ex vivo or in vivo) either directly or via a viral vector as described herein. Typically, the integration of the construct into the genomic DNA of the cells will be via homologous recombination, where the 5′ and 3′ DNA sequences in the promoter construct can serve to help integrate the modified promoter region via hybridization to the endogenous chromosomal DNA.

Uses Of AFTI Nucleic Acids And Polypeptides

AFTI nucleic acid molecules (including those that do not themselves encode biologically active polypeptides), may be useful as hybridization probes in diagnostic assays to test, either qualitatively or quantitatively, for the presence of an AFTI or Apo-A-1 DNA or corresponding RNA in mammalian tissue or bodily fluid samples.

The AFTI polypeptides may be used (simultaneously or sequentially) in combination with one or more cytokines, growth factors, antibiotics, anti-inflammatories, and/or chemotherapeutic agents as is appropriate for the indication being treated.

Other methods may also be employed where it is desirable to inhibit the activity of one or more AFTI polypeptides. Such inhibition may be effected by nucleic acid molecules that are complementary to and hybridize to expression control sequences (triple helix formation) or to AFTI mRNA. For example, antisense DNA or RNA molecules, which have a sequence that is complementary to at least a portion of the selected AFTI gene(s) can be introduced into the celf. Anti-sense probes may be designed by available techniques using the sequences encoding AFTI polypeptides disclosed herein. Typically, each such antisense molecule will be complementary to the start site (5′ end) of each selected AFTI gene. When the antisense molecule then hybridizes to the corresponding AFTI mRNA, translation of this mRNA is prevented or reduced. Anti-sense inhibitors provide information relating to the decrease or absence of an AFTI polypeptide in a cell or organism.

Alternatively, gene therapy may be employed to create a dominant-negative inhibitor of one or more AFTI polypeptides. In this situation, the DNA encoding a mutant polypeptide of each selected AFTI polypeptide can be prepared and introduced into the cells of a patient using either viral or non-viral methods as described herein. Each such mutant is typically designed to compete with endogenous polypeptide in its biological role.

In addition, an AFTI polypeptide, whether biologically active or not, may be used as an immunogen, that is, the polypeptide contains at least one epitope to which antibodies may be raised. Selective binding agents that bind to an AFTI polypeptide (as described herein) may be used for in vivo and in vitro diagnostic purposes, including, but not limited to, use in labeled form to detect the presence of AFTI polypeptide in a body fluid or cell sample. The antibodies may also be used to prevent, treat, or diagnose a number of diseases and disorders, including those recited herein. The antibodies may bind to an AFTI polypeptide so as to diminish or block at least one activity characteristic of an AFTI polypeptide, or may bind to a polypeptide to increase at least one activity characteristic of an AFTI polypeptide (including by increasing the pharmacokinetics of the AFTI polypeptide).

Effect of Apo-A-1 on IL-1β and TNF-α Production by Monocytes

Apo-A-I is a major component of HDL, which are macromolecular complexes of lipids and different amphipathic peptides termed apolipoproteins. The latter have a range of activities that allow HDL to accomplish different functions (Barter et al, 1996, Atherosclerosis 121: 1-12) that were initially attributed to their impact on blood lipid metabolism. More recently, it has become evident that HDL exhibit a range of activities (Calabresi et al, 1997, Curr. Opin. Lipidol. 8: 219-224; Miyazaki et al, 1995, J. Atheroscler. Thromb. 2: 30-36) including anti-inflammatory functions as illustrated in human volunteers where HDL was shown to down-regulate CD14 receptors on monocytes (Pajkrt et al, 1996, J. Exp. Med. 184: 1601-1608). HDL also displays anti-inflammatory functions by complexing lipopolysaccharide (LPS) thus contributing to a reduction in endotoxic activities (Baumberger et al, 1991, Pathobiology 59: 378-383). Although the mechanisms and physiological relevance of the anti-inflammatory functions of HDL have not been clarified, the results discussed in Example 1 below suggest that at least part of this activity may be attributable to the inhibition by apo-A-1 of T cell-signaling of monocytes.

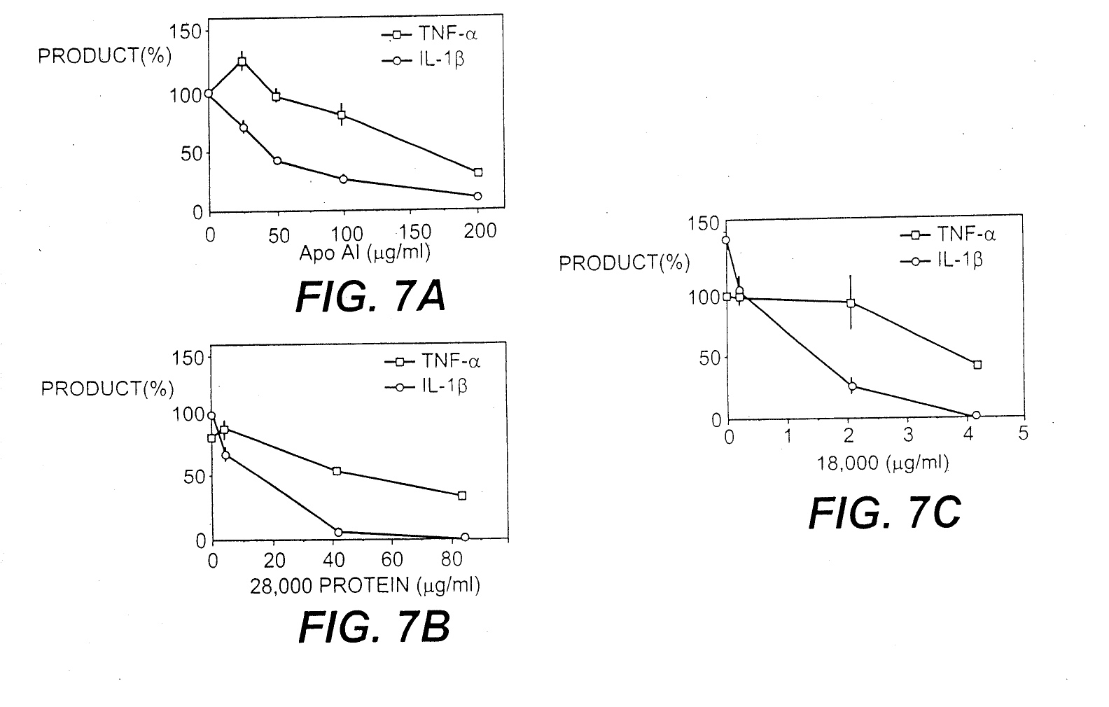

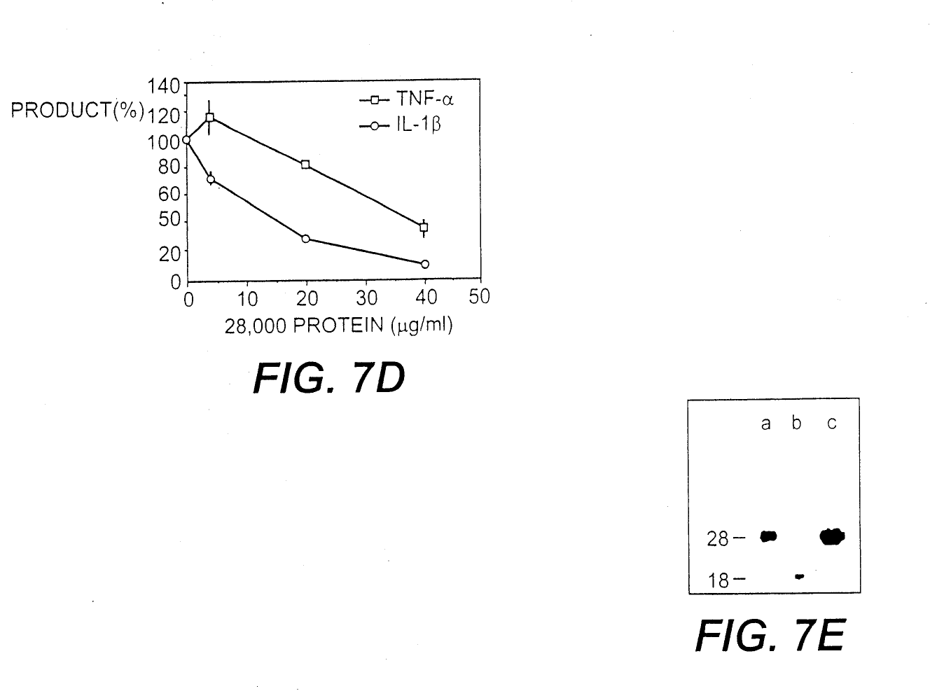

The data demonstrate that HDL interact with stimulated T cells via binding of apo-A-1. Such an interaction was described in a study demonstrating the presence of a specific HDL binding site on human lymphocytes that recognizes apo-A-1 as its ligand (Jurgens et al., 1989, J. Biol. Chem. 264: 8549-8556). Although this HDL receptor was not identified, it was claimed to be responsible for utilization of HDL lipids by T lymphocytes, when cultured in serum-free medium supplemented with HDL. In accordance with a more recent study (Hidaka et al., 1999, J. Lipid Res. 40: 1131-1139), the results discussed in Example 1 below reveal that HDL typically do not bind unstimulated T lymphocytes. These observations demonstrate that apo-A-1 associated HDL can interact with stimulated T cells. This interaction is likely to be involved in the inhibition of T cell-signaling of monocytes. Besides, according to flow cytometry analysis, HDL bind monocytes. This is consistent with published data showing the binding of HDL (HDL3) to monocytes in whole PBMC (Hidaka et al., 1999, J. Lipid Res. 40: 1131-1139). It is therefore very likely that HDL also affect monocyte activation by modulating directly the level of activation of the latter cells. This is confirmed by results shown in FIG. 7. Indeed, the premise that apo-A-1 added after the stimulus (membranes of stimulated HUT-78 cells or T lymphocytes) was still able to inhibit cytokine production activity suggests a direct effect of the inhibitor on monocytes/THP-1 cells. Although HDL binding or functional activity was not observed on resting THP-1 cells, the latter may express the HDL receptor once differentiated (activated) (Westman et al., 1995, Scand. J. Clin. Lab. Invest. 55: 23-33). Therefore, the results discussed in Example 1 below demonstrate that HDL-associated apo-A-l, according to certain embodiments, inhibits the production of proinflammatory cytokines upon activation of monocytes by contact with stimulated T cells through two different pathways; a major pathway being the blockade of the binding of the activating factor on T cells to its receptor on monocytes, thus inhibiting the expression of both TNF-α and IL-1β. Another pathway could be a direct effect of apo-A-I-associated HDL on monocytes through the binding to a receptor, thus directly inhibiting the production of cytokines. Although the latter effect remains to be demonstrated, these dual effects may explain the discrepancy in the extents of inhibition of TNF-α and IL-1β production in THP-1 cells.

Although the results in Example 1 did not identify the ligand(s) of apo-A-1 at the surface of stimulated T cells, it is unlikely that HDL receptor proteins such as SR-BI or CD36 are involved since the latter receptors do not display specificity for a particular apolipoprotein being able to interact with LDL (Krieger, M., 1999, Annu. Rev. Biochem. 68: 523-558; Fidge, N. H., 1999, J. Lipid Red. 40: 187-201). LDL does not display detectable inhibitory activity towards T cell-signaling of monocytes (data not shown). On the other hand, specific HDL binding proteins (HB₁ and HB₂) have been described that are expressed in rat liver plasma membrane and human blood monocytes (Hidaka et al, 1999, J. Lipid Res. 40: 1131-1139). HB₂ is homologous to ALCAM, a cell adhesion molecule belonging to the immunoglobulin superfamily (Fidge, N. H., 1999, J. Lipid Red. 40: 187-201). Since apo-A-1 has been involved in the binding of HDL to HB₂, it is possible that the HDL receptor on stimulated T cells might have some homologies with the HB₂ protein family. Recently, a protein, which was known as a high affinity receptor for the intestinal absorption of intrinsic factor-vitamin B12 complex (i.e., cubilin), was shown to display a high affinity for apo-A-1 facilitating HDL endocytosis (Kozyraki et al, 1999, Nat. Med. 5: 656-661). Whether the latter 460-kDa protein or HB₂ are expressed in stimulated T lymphocytes remains to be determined. The identification of apo-A-I/HDL receptor(s) on stimulated T cells might allow the elucidation of the mechanism of action of the inhibitor of T cell-signaling of monocytes.

The inhibition of T cell-signaling of monocytes might be important to maintain a low level of monocyte activation within the blood stream, although the static conditions used in this work might not reflect shear stress introduced by blood flow. Recently, it was shown that the inflammatory state in juvenile RA was associated with hypo-high density lipoproteinemia (Tselepis et al, 1999, Arthritis Rheum. 42: 373-383) and a significant decrease of apo-A-1 concentration in plasma of patients with this disease. In RA, controversial reports suggest that there are either no changes or a decrease in the plasma level of apo-A-1 (Ananth et al, 1993, Metabolism 42: 803-806; Lorber et al, 1985, Br. J, Rheumatol. 24: 250-255; Doherty et al, 1998, Electrophoresis 19: 355-363) or HDL cholesterol (Ananth et al, 1993, Metabolism 42: 803-806; Lorber et al, 1985, Br. J. Rheumatol. 24: 250-255; Joven et al, 1984, Arthritis Rheum. 27: 1199-1200). It is clear, however, that apo-A-1 is enhanced in synovial fluids of RA patients (Ananth et al, 1993, Metabolism 42: 803-806), the concentration of apo-A-1 being 10-fold lower in synovial fluid than plasma. The elevation of apo-A-1 in synovial fluid of RA patients was accompanied by an enhancement in cholesterol, suggesting an infiltration of HDL particles in the inflammatory site.

It is indeed common in inflammation that the activity of inflammatory factors, such as cytokines (IL-1β and TNF-α), and metalloproteinases is counteracted by specific inhibitors. In chronic inflammation, it is thought that regulatory mechanisms are overpowered by inflammatory factors. If, as the inventors hypothesize, T lymphocyte-signaling of monocytes is an important pro-inflammatory mechanism, there should be inhibitory factors to control this activity. It was demonstrated that HDL-associated apo-A-1 is one of these regulatory factors. The presence of apo-A-1 in synovial fluid of RA patients might be the response of the organism attempting to inhibit the inflammatory reaction. Variations in apo-A-1 concentration were also observed in another inflammatory disease of autoimmune etiology, systemic lupus erythematosus (SLE) (Lahita et al, 1993, Arthritis Rheum. 36: 1566-1574) in which apo-A-1 plasma concentrations were diminished. This decrease was associated with the presence of anti-apo-A-1 antibodies in 32% of patients (Dinu et al, 1998, G, Lupus 7: 355-360). This again indicates an anti-inflammatory function for apo-A-1.

There is a well established, inverse correlation of the concentration of HDL with the incidence of atherosclerotic disease. Increasing evidence strongly supports the contention that inflammatory responses are an integral part of atherosclerosis (Ross, R., 1999, N. Engl. J. Med. 340-115-126). Indeed, monocyte-macrophages and T lymphocytes are present at all phases of lesion development and the earliest lesion (fatty streak) is composed predominantly of macrophages and T lymphocytes (Stary et al, 1994, Circulation 89: 2462-2478). Therefore, T lymphocyte-signaling of monocytes might occur in this disease. Since the release of metalloproteinases by activated monocyte-macrophages is thought to weaken the plaque matrix and precipitate the acute-phase of plaque rupture and thrombus formation (Lee et al, 1997, Arterioscler. Thromb. Vase. Biol. 17: 1859-1867), HDL might exert protective functions at several levels in atherosclerosis, including the decrease of monocyte activation by T lymphocytes.

The results discussed herein identify an anti-inflammatory function for HDL-associated apo-A-1. According to certain embodiments, this might be a general mechanism of protection against overactivation of monocyte-macrophages in inflammatory conditions where stimulated T lymphocytes are found in the blood circulation as well as an important counter-regulatory mechanism when plasma proteins leak into the inflamed tissue. A new concept emerging from the present results is the importance of “negative” acute-phase proteins such as apo-A-1 as anti-inflammatory factors The following examples are intended for illustration purposes only, and should not be construed as limiting the scope of the invention in any way.

### EXAMPLE 1

### Using Apo-A-1 to inhibit TNF-α and IL-1β production in human monocytes that were stimulated through contact with T lymphocytes.

Materials and Methods

Materials and reagents. Phaseolus vulgaris leucophytohemagglutinin (PHA) (E-Y Laboratories Inc., San Mateo, Calif.); phorbol myristate acetate (PMA), paraformaldehyde, phenylmethylsulfonyl flouride (PMSF), pepstatin A, leupeptin, iodoacetamide, polymyxin B sulfate, neuraminidase, and bovine serum albumin (Sigma Chemicals Co., St. Louis, Mo.); RPM1-1640, phosphate-buffered saline without Ca²⁺ and Mg²⁺ (PBS), fetal calf serum (FCS), penicillin, streptomycin and L-glutamine (Gibso, Paisley, Scotland) were purchased from the designated suppliers. All other reagents were of analytical grade or better.

Human Serum. Pooled human serums (HS) were obtained from the Blood Transfusion Center of the University Hospital of Geneva.

T cells and preparation of T cell plasma membranes. HUT-78, a human T cell line (Gazdar et al, 1980, Blood 55: 409-417), was obtained from the ATCC (Manassas, Va.). Cells were maintained in RPMI-1640 medium supplemented with 10% heat-inactivated FCS, 50 μg/ml streptomycin, 50 lU/ml penicillin and 2 mM L-glutamine (complete RPMI medium) in a 5% CO₂-air humidified atmosphere at 37° C. HUT-78 cells (1×10⁶ cells/ml) were stimulated for 6 hours by PHA (1 μg/ml) and PMA (5 ng/ml). Stimulated HUT-78 cells were either fixed with 1% paraformaldehyde (Vey et al, 1992, J. Immunol. 149: 2040-2046; Isler et al, 1993, Eur. Cytokine Netw. 4: 15-23) or their plasma membranes prepared as previously described (Burger et al, 1998, Arthritis Rheum. 41: 1748-1759). T lymphocytes were obtained from buffy coats of healthy donors as previously described (Vey et al, 1992, J. Immunol. 149: 2040-2046), and contained 94-98% CD2+, 83-94% CD3+, and <2% CD14⁺ as assessed by flow cytometry. T lymphocytes were stimulated for 48 hours by PHA (1 μg/ml) and PMA (5 ng/ml), washed thoroughly and fixed with 1% paraformaldehyde as previously described (Vey et al, 1992, J. Immunol. 149: 2040-2046; Isler et al, 1993, Eur. Cytokine Netw. 4: 15-23). Peripheral blood mononuclear cells (PBMC) were obtained from buffy coats of healthy donors by density centrifiguation on Ficoll-Paque (Amersham-Pharmacta, Uppsala, Sweden).

Monocytes and monocytic cells. The human monocytic cell line THP-1, derived from a patient with acute monocytic leukemia (Tsuchiya et al, 1980, Int. J. Cancer 26: 171-176) was obtained from the ATCC (Manassas, Va.). Peripheral blood monocytes were obtained as described (Armant et al, 1995, J. Immunol. 155: 4868-4875).

PMBC Cultures. PMBC were cultured in 96-well culture plates at a density of 4×10⁵ cells per 200 μl per well in the presence of the indicated stimulus for 48 hours (cytokine production) or for 72 hours (proliferation). For proliferation assays, ³H-thymidine was added 24 hours before cell harvesting.

Protein concentration and N-terminal microsequencing. The protein concentrations were determined by the method of Bradford. The purified inhibitory fraction was subjected to 10% SDS-PAGE, transfered to PVDF membrane, and visualized by Coomassie blue staining. The M, 28 kDa band was excised and N-terminal sequence analysis was performed on a Procise 494-HT protein sequencer polypeptide(Perkin-Elmer, Foster City, Calif.).

Activation of THP-1 cells and monocytes. THP-1 cells (5×10⁴ cells/well) or monocytes (8×10⁴ cells/well) were dispensed onto 96-well culture plates (Falcon, Becton Dickinson UK Ltd., Plymouth, UK) and activated by the indicated stimulus in a total volume of 200 μl complete RPMI medium in the presence or absence of the indicated inhibitor. After 48 hours, culture supernatants were analyzed for their contents in TNF-α and Iβ-1J3 as previously described (Vey et al, 1992, J. Immunol. 149: 2040-2046; Isler et al., 1993, Eur. Cytokine Netw. 4: 15-23).

Isolation of serum HDL by high-density ultracentrifugation. HS lipoproteins were isolated according to Havel et al, 1955, J. Clin. Invest. 34: 1345-1353. Briefly, to remove the chylomicrons, HS was centrifuged for 45 minutes at 20,000 rpm using a Beckman JA 20.1 rotor. Serum free of cylomicrons was then centrifuged for 24 hours and 37 minutes at 50,000 rpm. The upper phase containing very low density lipoproteins (VLDL) was discarded. The lower phase was adjusted to a density of 1.063 g/ml by the addition of solid NaBr and centrifuged for 24 hours 37 minutes at 50,000 rpm. The low density lipoproteins (LDL) that were recovered in the upper phase were discarded. The lower phase was adjusted to d=1.23 g/ml by the addition of solid NaBr and centrifuged for 60 hours 47 minutes at 45,000 rpm. High density lipoproteins (HDL) were recovered in the upper phase, while the lower phase contained the remaining serum proteins. All ultracentrifugations were carried out at 4° C. using a Beckman 50.2. Ti rotor.

The recovered lipoprotein and protein fractions were dialyzed against PBS and tested for their inhibitory activity. Briefly, THP-1 cells (5×10⁴ cells/well) or monocytes (8×10⁴ cells/well) were dispensed onto 96-well culture plates (Falcon, Becton Dickinson UK Ltd., Plymouth, UK) and activated by membranes isolated from stimulated T cells in a total volume of 200 μl complete RPMI medium in the presence of different dilutions of fraction. After 48 hours, culture supernatants were analyzed for their contents in TNF-α and IL-1β as previously described (Vey et al., 1992, J. Immunol. 149: 2040-2046; Isler et al., 1993, Eur. Cytokine Netw. 4: 15-23).

Flow cytometry. HDL were labeled with fluorescein thiocyanate (FITC-HDL) as polypeptide described (Hidaka et al, 1999, J. Lipid Res. 40: 1131-1139). The binding of FITC-HDL to cells was analyzed by direct flow cytometry on a flow cytometer (EPICS, Coulter Electronics Inc., Hialeah, Fla.) essentially as described previously (Deage et al, 1998, M. Eur. Cytokine Netw. 9: 663-668). The mean fluorescence intensity was recorded upon gating of living cells and expressed in arbitrary units of 4 decade logarithmic scale. The percentage of positive cells was based on the percentage of fluorescent events exceeding an unconjugated FITC control.

HDL delipidation. The extraction of HDL apolipoproteins was performed as described (Osborne, J. C. Jr., 1986, Meth. Enzymol. 128: 213-222). Briefly, 1 volume of HDL isolated by ultracentrifugation was slowly added to 12 volumes of ice-cold methanol upon constant stirring. Then 28 volumes of ice-cold diethylether were added to the solution. After 10 minutes stirring on ice, the mixture was centrifuged at 500×g for minutes. The protein pellet was resuspended in 40 volumes of diethylether. After 10 minutes stirring on ice, the mixture was centrifuged as above. The pellet was recovered and dried under nitrogen flux. To minimize aggregation, the delipidated HDL lipoproteins were solubilized at 2 mg protein/ml in 0.1 M Tris-HCl, pH 7.4, containing 0.1 M NaCl, 1 mM NaN₃, 1 mM EDTA, and 2 M guanidinium chloride and then dialyzed against PBS.

Treatment of HDL with proteinase K. HDL (100 μg protein) were incubated in the presence or absence of one unit of proteinase K linked to agarose beads (Sigma Fine Chemicals, St. Louis, Mo.) in a final volume of 200 μl PBS at 37° C. for 1 hour. The proteolytic reaction was stopped by centrifugation and its efficacy assessed by SDS-PAGE.

Electroelution of HDL proteins. HDL proteins were isolated by electroelution from gel slices essentially as previously described (Burger et al, 1990, J. Neurochem. 54: 1569-1575). Briefly, 1 mg of delipidated HDL protein was subjected to SDS-PAGE under nonreducing conditions. The gel was stained with 0.3 M CuCl₂ (Lee et al, 1987, Anal. Biochem. 166: 308-312). The detected bands (Mᵣ=56,000-66,000, 50,000, 28,000, and 18,000) were cut out, destained, and electroeluted for hours in 50 mM Tris-HCl and 384 mM glycine (pH=8.3) containing mM EDTA, and 0.1% SDS. SDS polypeptide was removed by precipitating the electroeluted proteins in acetone. Proteins were lyophilized and resuspended in 0.1 M Tris-HCl, pH 7.3, containing 0.1M NaCl, 1 mM NaN₃, 1 mM EDTA, and 2 M guanidinium chloride and then dialyzed in PBS. Alternatively, proteins from delipidated HDL were solubilized in 0.1 M Tris-HCl pH 7.4 containing 0.1 M NaCl, 1 mM NaN₃, 1 mM EDTA, and 2 M guanidinium chloride and subjected to gel filtration on Superdex S75 (75×1.6 cm, Pharmacia) equilibrated in the same buffer. Fractions corresponding to Mᵣ=28,000 were pooled, concentrated and dialyzed in PBS for testing their inhibitory activity. Fractions were analyzed by Western blot for their apo-A-1 content using a mouse monoclonal antibody from Calbiochem-Novabiochem Corp. (La Jolla, Calif.), catalog number 178472.

mRNA quantification. Total RNA was isolated from THP-1 cells and from monocytes with TRIzol™ reagent (Life Technologies) according to the manufacturer's procedure. Two micrograms and 10 μg of total RNA were used to quantify mRNAs in monocytes and in THP-1 cells, respectively, using a commercially available “RNase protection assay system” kit with hck2 template set (PharMingen, San Diego, Calif.) to which an anti-sense riboprobe for TNF-α was added. The TNF-α anti-sense riboprobe was obtained from a TNF-α cDNA template prepared with SP6 RNA polymerase after linearizing the pSP65/hTNF plasmid provided by Dr. C. V. Jongeneel (Ludwig Institute for Cancer Research, Lausanne, Switzerland).

Results

Human serum inhibited T cell-signaling of both monocytes and THP-1 cells.

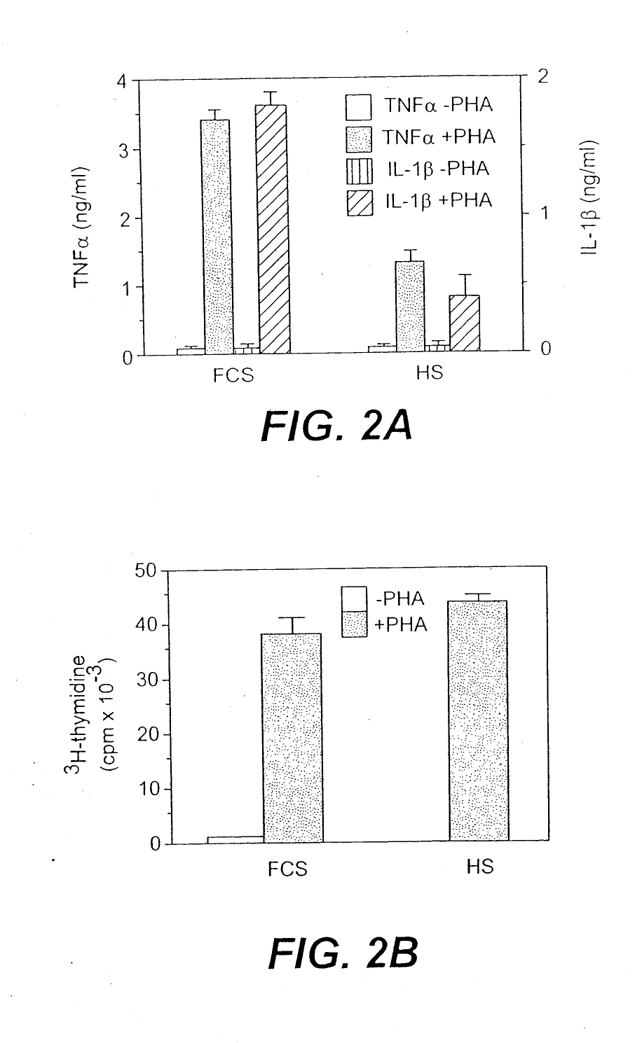

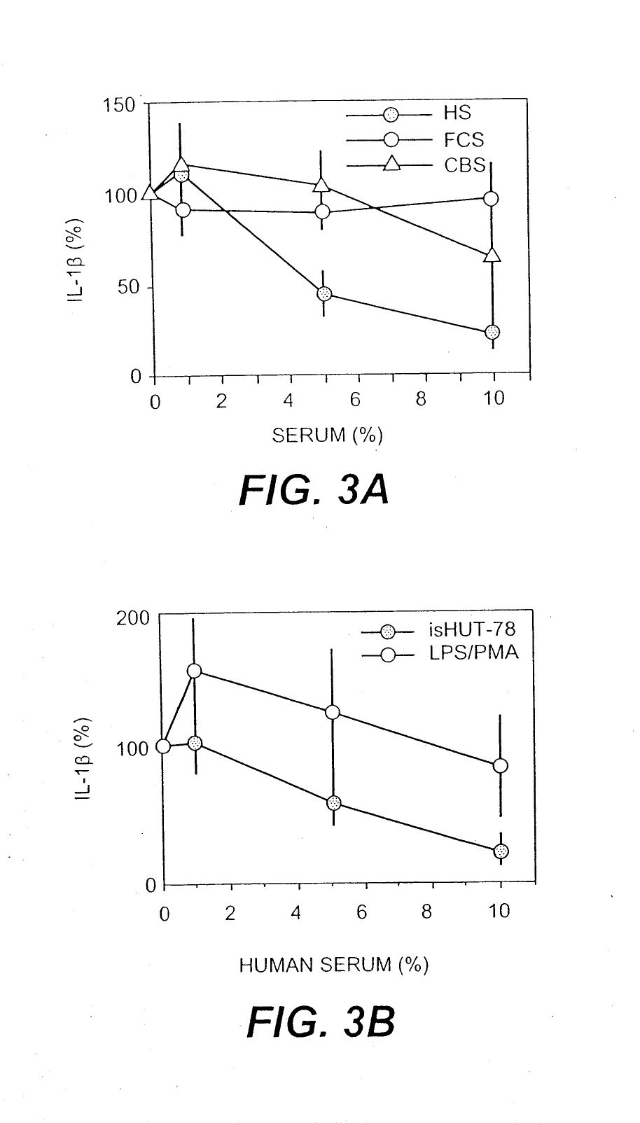

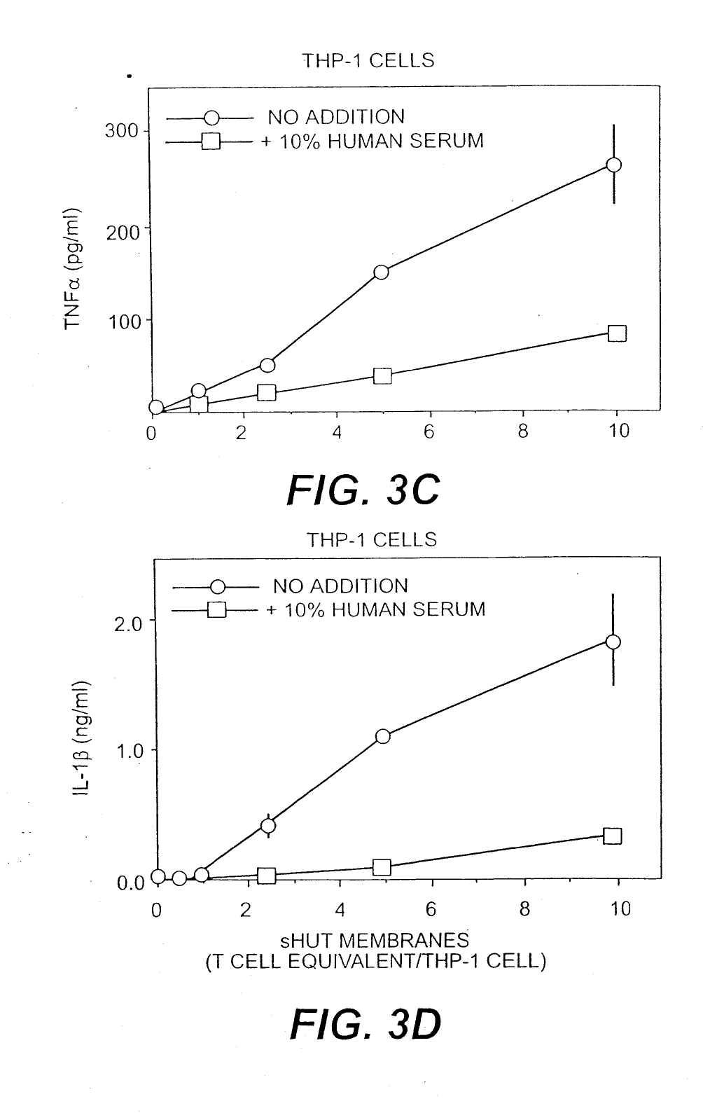

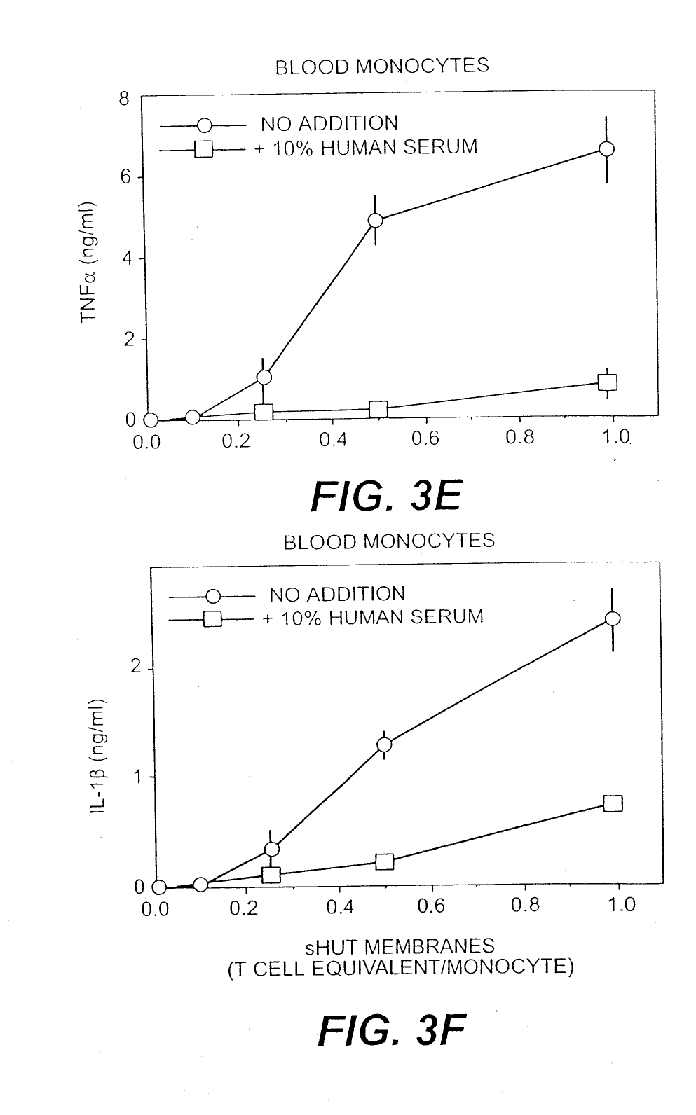

In order to determine whether human serum (HS) displayed anti-inflammatory activity by inhibiting cytokine production, unseparated PBMC were stimulated by PHA in medium supplemented with either 10% FCS or HS. The production of both TNF-α and IL-1β was inhibited in PBMC cultured with HS as compared with PBMC cultured with FCS (FIG. 2A), but cell proliferation was similar in HS and FCS (FIG. 2B). Since it was likely that cytokine production by PBMC was increased in monocytes by direct contact with stimulated T lymphocytes, the inhibitory effect of HS was examined in several culture systems in which either fixed, stimulated T lymphocytes or HUT-78 cells or plasma membranes from HUT-78 cells were added to PBMC or THP-1 monocytic cells. The induced production of TNF-α and IL-1β was measured (FIG. 3).

T lymphocytes isolated from peripheral blood were stimulated with PHA and PMA, fixed, and added to THP-1 cells in the presence of either human serum (HS) or fetal calf serum (FCS), i.e., a final serum concentration of 20%. In these conditions, the production of IL-1 (3 by THP-1 cells was inhibited in a dose-dependent manner by HS but not by FCS (FIG. 3A). This demonstrates the presence of an inhibitory activity in HS. Human cord blood serum (CBS) was only slightly inhibitory at 10% concentration (FIG. 2A), suggesting that the inhibitory activity was present only in adult serum.

To ascertain that the inhibition was specific for contact activated monocytes, THP-1 cells were stimulated by either fixed, stimulated HUT-78 cells or by LPS and PMA in the presence of increasing concentrations of HS. IL-1β produced upon contact of THP-1 cells with fixed, stimulated HUT-78 cells was inhibited in a dose-dependent manner by HS, whereas that induced by LPS and PMA was stimulated or unchanged depending on HS concentration (FIG. 3B). This demonstrates that the inhibitory activity of HS was directed to T ceil signaling of THP-1 cells and that freshly isolated T lymphocytes and the HUT-78 cell line expressed an activitating factor(s) upon stimulation (FIG. 3, A and B).

Plasma membranes of stimulated T cells induced both TNF-α and IL-1β production in THP-1 cells and in freshly isolated monocytes (FIG. 3, C˜F). However, monocytes were more sensitive to contact activation with membranes of stimulated HUT-78 cells than THP-1 cells, which required a 10-fold higher amount of membranes for activation- Indeed, IL-1β and TNF-α production was triggered by isolated membranes at an amount equivalent to a cellular ratio as low as 0.1 stimulated T cell/monocyte, whereas in THP-1 cells cytokine production was observed at 2 stimulated T cell/THP-1 cell (FIG. 3, C-F). Similar levels of IL-1β were induced in both type of cells (FIG. 3, D and F), whereas levels of TNF-a were 20-fold lower in THP-1 cells than in monocytes.

Therefore, the degree of inhibition for different cytokines depended on the target polypeptidecell type. Indeed, in monocytes the production of TNF-α and IL-1β was inhibited to a similar extent reaching about 85% and about 91% inhibition, respectively, in membrane doses ranging from 0.25 to 1.0 T cell equivalent/monocyte. In THP-1 cells, TNF-α production was inhibited less than that of IL-1β reaching about 69% and about 89%, respectively, at membrane doses ranging from 2.5 to 10 T cell equivalent/monocyte. This indicates that the inhibition of TNF-α production in THP-1 cells was less efficient than that of IL-β in contrast with monocytes in which TNF-α and IL-1β were inhibited to a similar extent. These data confirmed the high potency of direct contact with stimulated T cells in triggering TNF-α and IL-1β production by monocytes and confirmed the hypothesis that inhibitory mechanisms or factors were present in HS. To further identify the HS inhibitory component and the mechanism of inhibition, THP-1 cells and membranes from stimulated HUT-78 cells were used.

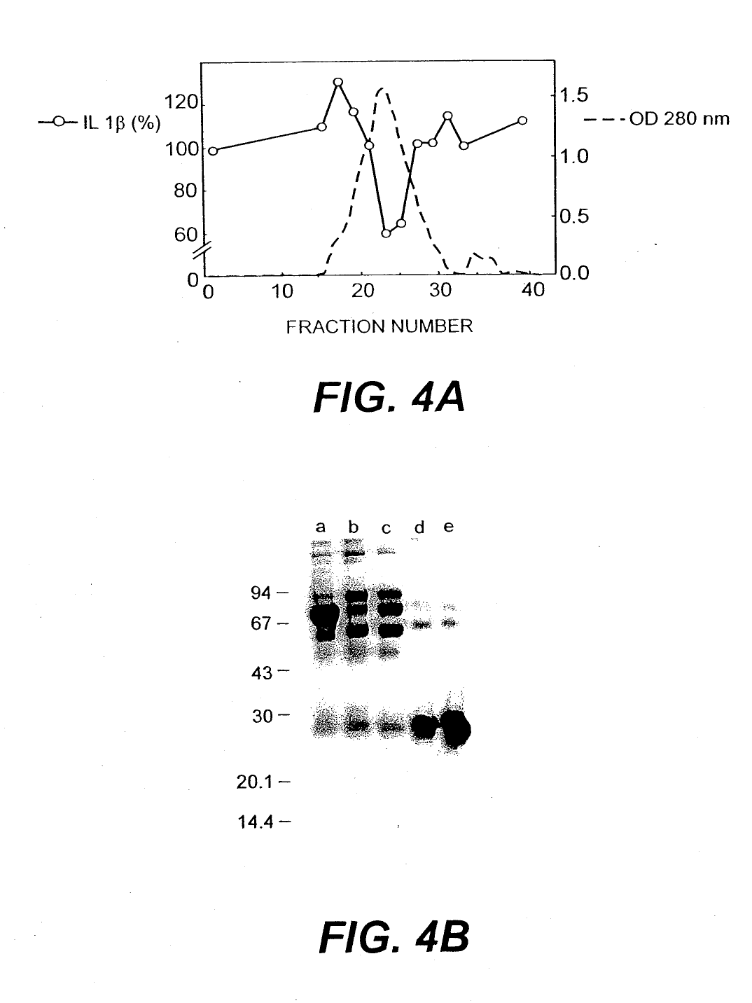

Isolation of HS factor inhibiting T cell-signaling of monocytes. To identify the inhibitory factor, HS was fractionated by serial chromatography on Blue Sepharose® fast flow, Q Sepharose®M fast flow, phenyl Sepharose® 6 fast flow, and Superdex® 200 (Pharmacia, Uppsala, Sweden). The inhibitory activity was recovered in fractions 23-26 (FIG. 4A), i.e., with a M_{f}=179×10³±46×10³ (mean±SD, n=4 inhibitory fractions). Upon analysis by sodium dodecyl sulfate polyacrylamide gel electrophoresis (SDS-PAGE), the inhibitory activity correlated with the enrichment of a 28 kDa protein band (FIG. 4, Table 1). N-terminal microsequencing proved this band to be apolipoprotein (apo) A-1, a major protein component of high density lipoprotein (HDL). This explained the discrepancy in Mᵣanalysis between gel filtration and SDS-PAGE analysis where HDL and apo-A-1 were separated, respectively (FIG. 4, A and B). These data demonstrated that the inhibitor was contained in HDL particles.

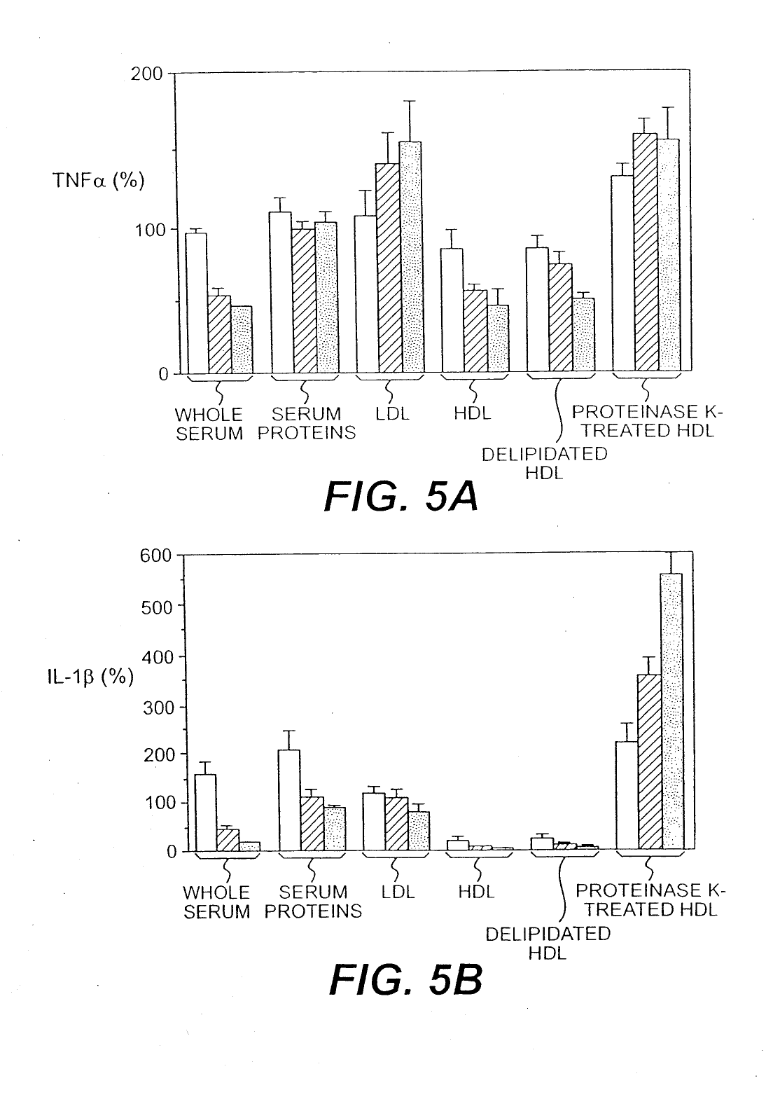

Protein components of HDL inhibited T cell-signaling of monocytes. To determine which HDL component was inhibitory, HS was fractionated by serial high-density centrifugations (Havel et al, 1955, J. Clin. Invest. 34: 1345-1353) to separate lipoproteins from serum proteins. Isolated lipoproteins and serum proteins were dialyzed against PBS and tested for their inhibitory activity in THP-1 cells activated by membranes of stimulated HUT-78 cells. The inhibitory activity was recovered in HDL fractions whereas LDL and serum proteins, which were recovered using the same polypeptideultracentrifugation (d=.1.23) as HDL, were not inhibitory (FIG. 5). HDL fractions were subjected to either delipidation or proteolytic treatment with proteinase K to determine which component of HDL was the inhibitor, i.e., a protein or a lipid. HDL proteins obtained by diethylether/methanol treatment displayed a high inhibitory activity whereas HDL lipids obtained after proteolytic digestion with proteinase K were no longer inhibitory (FIG. 5). The production of both TNF-A and IL-1β was inhibited by HDL proteins (FIG. 5, A and B). This confirms that the HS inhibitor was a protein component of HDL.

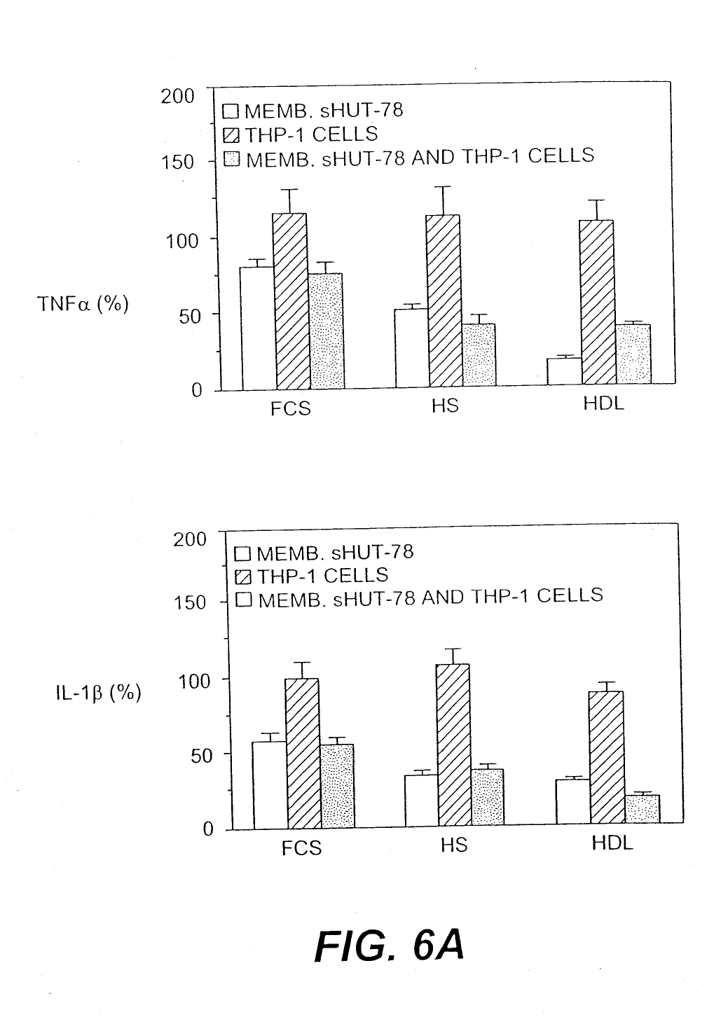

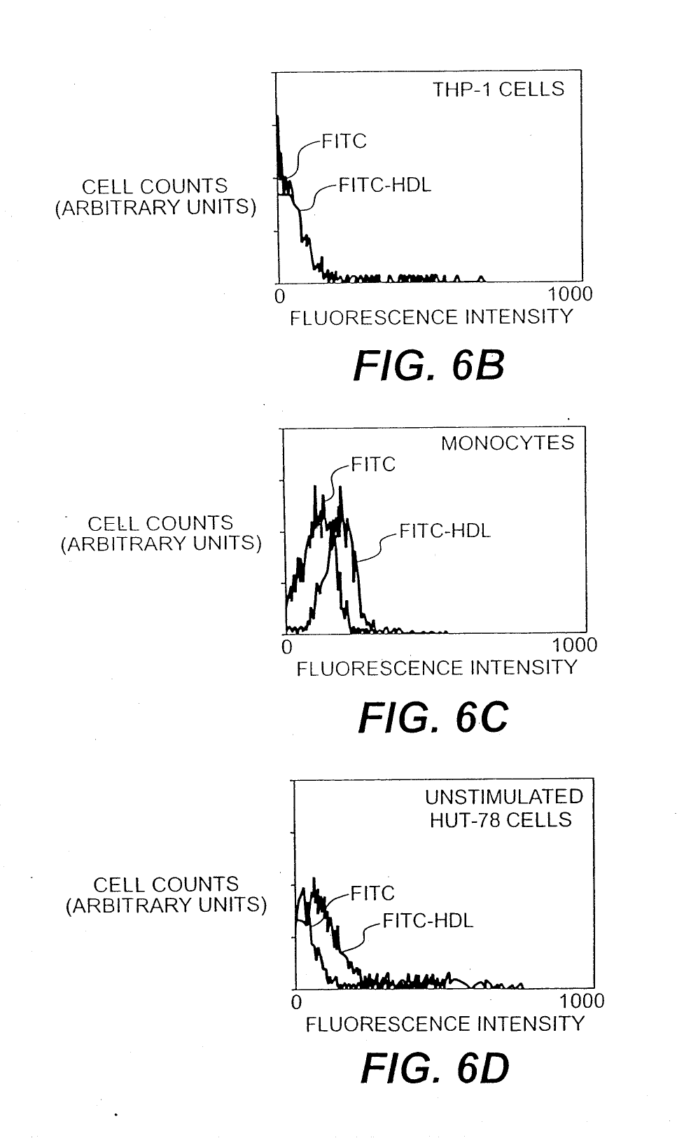

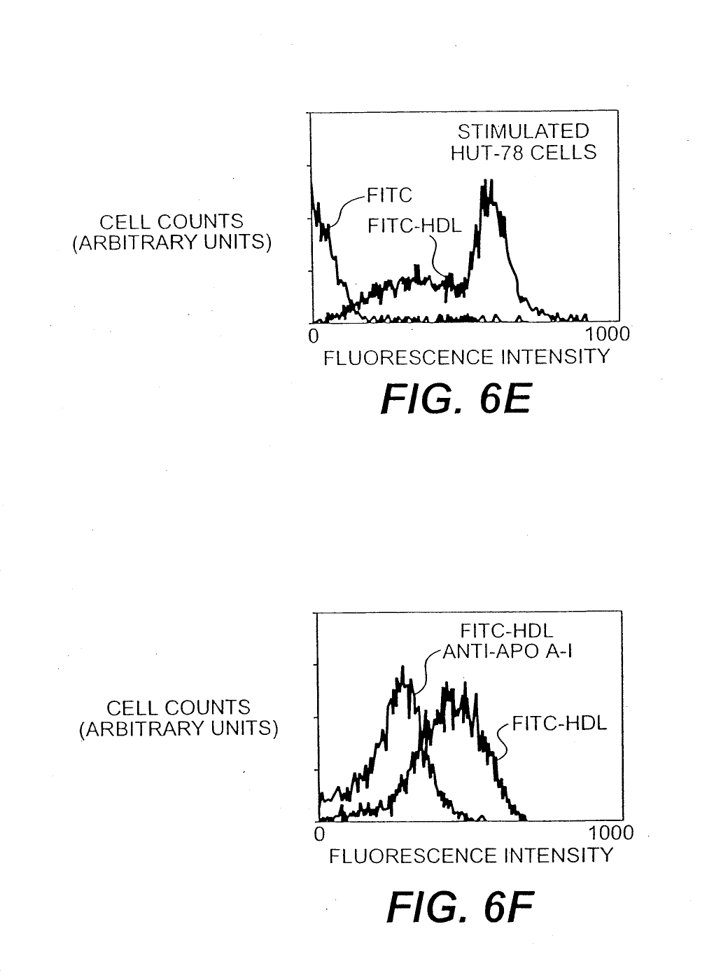

Apo-A-1 is the HS inhibitor of T cell-signaling of monocytes. To determine whether apo-A-1 displayed the inhibitory activity, commercially available, purified apo-A-1 (Sigma Fine Chemicals, St. Louis, Mo.) was tested. Apo-A-1 inhibited the production of both TNF-α and IL-β in THP-1 cells activated by membranes of HUT-78 cells in a dose-dependent manner (FIG. 6A). As already evident from FIG. 3, TNF-α production was less inhibited than IL-1β production. Since the apo-A-1 preparation contained 3% unidentified contaminants (according to the supplier), it had to be ascertained that the inhibition was indeed due to apo-A-1. Proteins from delipidated HDL were subjected to preparative SDS-PAGE. After copper-straining, bands (Mᵣ: 56,000-66,000, 50,000, 28,000, and 18,000) were excised and electroeluted. The inhibitory activity was recovered in the Mᵣ=28,000 and Mᵣ=18,000 bands (FIG. 6, B and C). The double Mᵣ=56,000-66,000 band, which contained apo-A-1 aggregates as assessed by Western blotting, did not show significant inhibitory activity (not shown). Production of both IL-1β and TNF-α was inhibited by the electroeluted proteins. TNF-α was inhibited to a lower exent than IL-1β, confirming the results from FIG. 2. All inhibitory fractions contained apo-A-I as demonstrated by Western blot analysis (FIG. 6E), pointing to apo-A-1 as the inhibitor of T cell-signaling of monocytes. Indeed, it is very unlikely that another HDL apolipoprotein would display the same behavior as apo-A-I in terms of size of protein and proteolytic fragment (FIG. 6E, lanes a and b). Alternatively, proteins from delipidated HDL were subjected to gel filtration on Superdex S75. Pooled fractions corresponding to Mᵣ=28,000±10,000 displayed the inhibitory activity (FIG. 6D). These fractions contained apo-A-I as determined by Western blot analysis (FIG. 6E, lane c), further confirming that apo-A-I was the inhibitor.

HDL interact with stimulated T cells through apo-A-I binding. To determine whether the inhibitory activity of HDL was due to its potential binding to stimulated T cell membranes or to THP-1 cells, either THP-1 cells or membranes isolated from stimulated HUT-78 cells were preincubated in the presence or absence of FCS, HS, or isolated HDL. After washing, the residual activation capacity of membranes from stimulated HUT-78 cells on THP-1 cells was assessed. The inhibition of IL-1β production was observed only when membranes of stimulated HUT-78 cells were incubated with HS or HDL (FIG. 7A). Incubation of THP-1 cells with either FCS, HS, or HDL did not appear to inhibit the production of IL-1β (FIG. 7A). These results demonstrate that the inhibitory activity of HS and HDL was mainly directed to the activating factor(s) expressed at the surface of stimulated T cells.

To further confirm that the inhibitory factor(s) interacted with surface factors on stimulated T cells, isolated HDL was labeled with fluorescein isothiocyanate (FITC) and its binding to different cell types assessed by flow cytometry. No binding of FITC-HDL was observed on THP-1 cells (FIG. 7B), whereas fluorescence of monocytes was slightly enhanced when incubated with FITC-HDL as compared to unconjugated FITC control (FIG. 7C). A low level of binding of FITC-HDL to unstimulated HUT-78 cells was observed, whereas stimulated HUT-78 cells bound FiTC-HDL, displaying 2 fluorescent peaks, suggesting the presence of at least 2 different HDL binding sites (FIG. 7, D and E). At a lower FITC-HDL concentration, only one fluorescent peak was observed. In the presence of anti-apo-A-1 antibodies, a shift toward lower fluorescence intensity was observed, demonstrating that HDL interacted with stimulated T cells via apo-A-I specific binding (FIG. 7F). Together these results show that HDL interacted preferentially with stimulated T cells, implying that the inhibitory activity involving apo-A-1 was directed to surface factors on T cells.

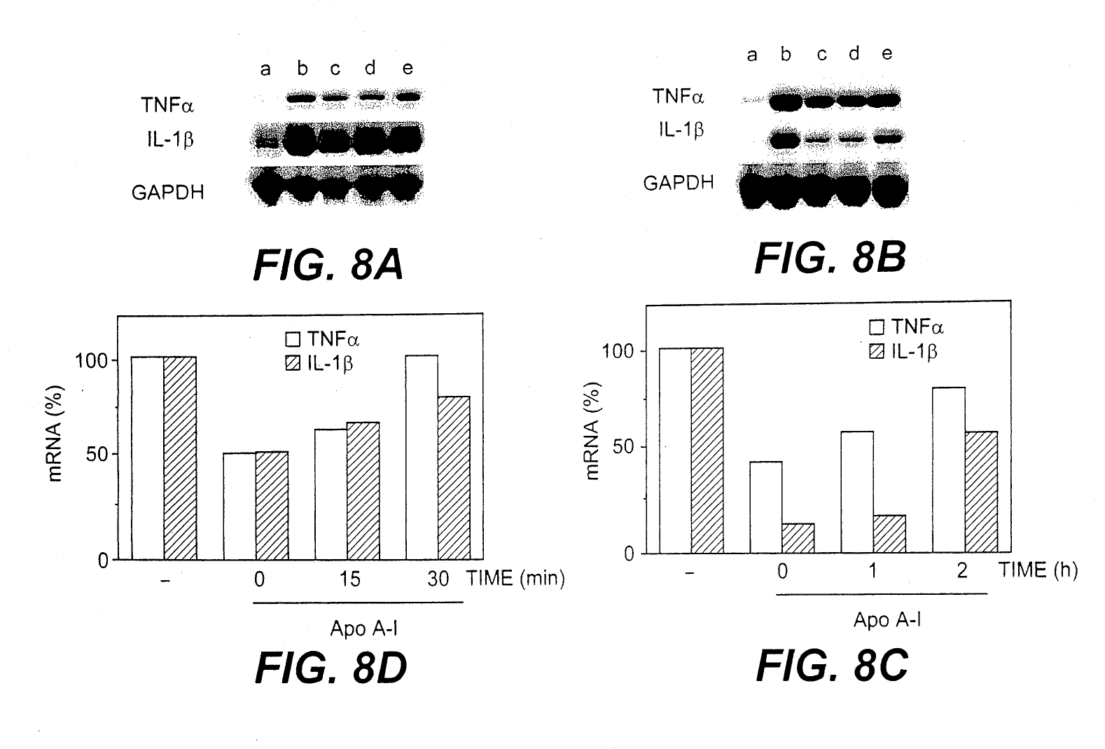

Optimal inhibition of steady state mRNA levels was only observed with the addition of apo A-1 simultaneously or shortly after the stimulus. To further elucidate the mechanism of apo A-1, the inhibitory effect of isolated apo A-1 on steady-state levels of TNF-α and IL-1β mRNA was assessed. THP-1 cells were stimulated with membranes of stimulated HUT-78 cells in the presence of apo-A-1 added at different times. Apo-A1 diminished the steady-state levels of TNF-α and IL-1β mRNA in THP-1 cells activated with membranes of stimulated HUT-78 cells (FIG. 8, A and C). The inhibition of TNF-α mRNA was less pronounced than that of IL-1β mRNA, correlating with the data obtained on the protein production level. Inhibition of TNF-α mRNA was only observed when apo-A-1 was present at time 0 or shortly thereafter, suggesting that the inhibition of TNF-α production was due to the interaction of apo-A-1 with the activating factor on stimulated HUT-78 cells. Similar results were obtained with PBMC activated by membranes of stimulated T lymphocytes (FIG. 8, B and D), in which steady-state levels of both TNF-α and IL-1β mRNA were diminished by apo-A-1 or HS although mRNA induction was induced more rapidly in monocytes than in THP-1 cells. In monocytes, the inhibition of steady-state mRNA levels also was optimal when apo A-1 was added together with the membranes or shortly therafter (FIG. 8D). No inhibition was observed if apo A-1 was added 30 minutes after activation. The latter results demonstrate that apo A-1 inhibited contact-mediated activation of monocytes regardless of cell type, confirming the data in FIG. 3.

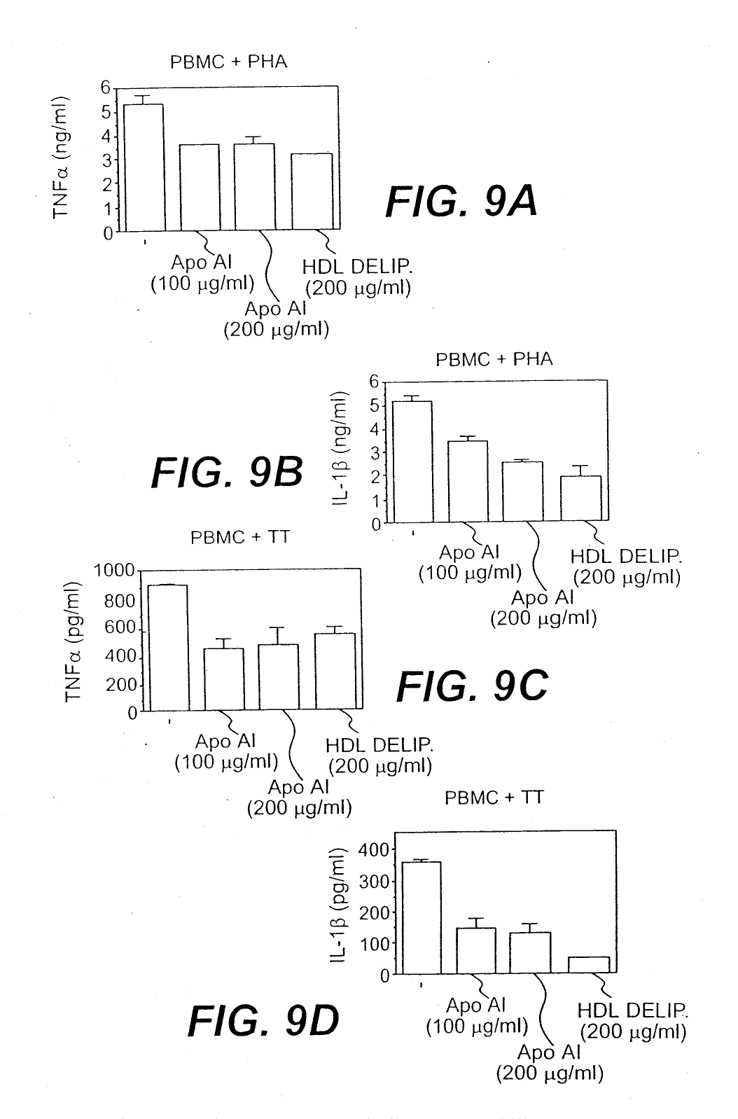

Apo A-1 inhibits TNF-a and IL-ip production in antigen-activated PBMC. To confirm that apo A-1 was the factor responsible for the inhibition of cytokine production in PBMC stimulated by PHA, PBMC were stimulated by either PHA or tetanus toxoid (TT) in the presence or absence of apo A-1 and delipidated HDL. Although cytokine production was lower in TT- than in PHA-stimulated PBMC, TNF-α and IL-1β production was inhibited by either apo A-1 or by delipated HDL regardless of the stimulus (FIG. 9). TNF-α production was inhibited to a lesser extent than that of IL-1β, confirming the results shown in FIG. 2. This demonstrates that (i) the inhibition of cytokine production by unfractionated HS shown in FIG. 2 was due to apo A-1, and (ii) that apo A-1 inhibits monocyte activation regardless of the T cell stimulus, suggesting that antigen-specific stimulation and mitogen stimulation induced similar activating factors on T lymphocytes, although inducing different levels of expression. Furthermore, these results confirm that TNF-α and IL-1 (3 were induced in PBMC only when monocytes were contacted with stimulated T cells.

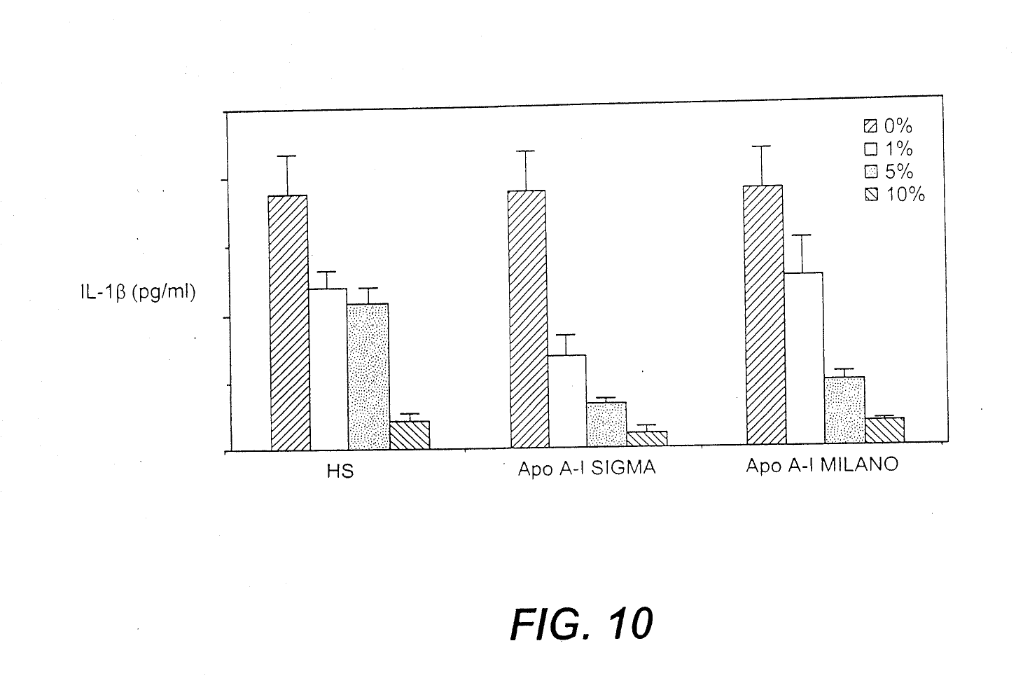

Recombinant mutant apo A-l_{Mi}iano displays the inhibitory activity. To exclude the possibility that a minor contaminant protein having similar physico-chemical properties to apo A-1 could be the inhibitor, the activity of a recombinant mutant of apo A-1 (kind gift of Professor G. Franceschini, Milano, Italy) also was tested in the contact assay. The mutant protein apo A-1_{Milano} (apo A-I_{M}) differs from wild-type apo A-1 by an Arg₁₇₃/Cys substitution, leading to the formation of disulfide-linked dimers (Calabrezi et al., 1994). In general, the in vitro and in vivo features of apo A-I_{M} differ only slightly from wild type apo A-1. HDL particles containing A-I_{M}/A-I_{M} are more efficient than those containing apo A-1 in promoting cholesterol efflux from cells, are less effective for the lecithin cholesterol acetyl transferase (LCAT) enzyme, and are equally effective in inhibiting the cytokine-induced expression of adhesion molecules on endothelial cells (Franceschini et al., 1998). As shown in FIG. 10, recombinant mutant apo A-1 inhibits IL-1β production in contact activated monocytes as efficiently as purified commercially available apo A-1 confirming again that apo A-1 is the inhibitor.

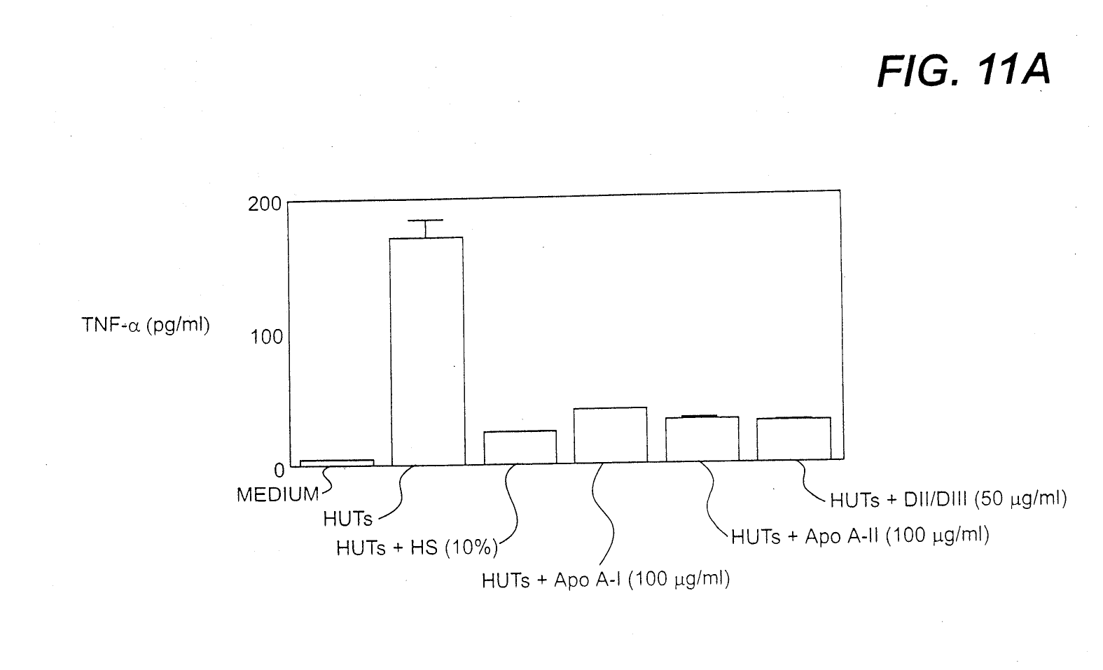

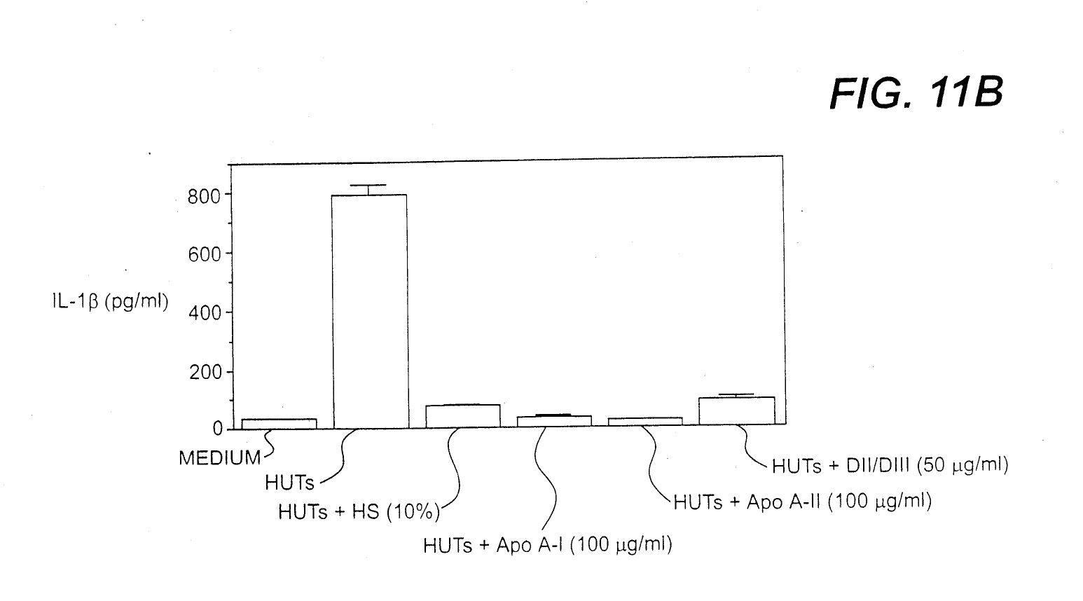

A fragment containing apo A-1 domains II and III displays the inhibitory activity. THP-1 cells were activated by contact with membranes of stimulated HUT-78 cells (HUTs) in the presence of 10% human serum, 100 ug/ml apo A-1, 100 μg/ml apolipoprotein A-1I, or 50 μg/ml of a fragment containing domains II and III of apo A-1 (DII/DIII). As shown in FIG. 11, DII/DIII inhibited both IL-1β and TNF-α production by the THP-1 cells.

The results show an anti-inflammatory activity elicited by apo-A-1, i.e., by a “negative” acute-phase protein (Gabay et al., 1999, N. Engl. J. Med. 340: 562-569). This activity seems to be specifically directed to T cell-signaling of monocytes since activation of monocytic cells by other stimuli (i.e. LPS and PMA) typically was not affected by the HS inhibitor, i.e., apo-A-1. Furthermore, membranes of stimulated HUT-78 cells treated with HS or HDL displayed a lower cytokine-inducing capacity than untreated membranes, whereas HDL treatment of THP-1 cells had no observed effect. This demonstrates that the inhibitory activity was mainly directed toward the activating factors expressed at the surface of stimulated T cells. Although the mechanism of action of apo-A-1 is not fully elucidated, and the present invention is not limited to any particular mechanism, the present results suggest that apo-A-1, according to certain embodiments, might exert its activity by specifically hampering the interaction between T cell membrane activating factor(s) and its (their) respective receptor(s) on monocytes.

### EXAMPLE 2

### Production Of AFTI Polypeptides A. Bacterial Expression

PCR is used to amplify template DNA sequences encoding an AFTI polypeptide using primers corresponding to the 5′ and 3′ ends of the sequence. The amplified DNA products may be modified to contain restriction enzyme sites to allow for insertion into expression vectors. PCR products are gel purified and inserted into expression vectors using standard recombinant DNA methodology. An exemplary vector, such as pAMG21 (ATCC No. 98113) containing the lux promoter and a gene encoding kanamycin resistance is digested with BamHI and Ndel for directional cloning of inserted DNA. The ligated mixture is transformed into an E. coli host strain by electroporation and transformants are selected for kanamycin resistance. Plasmid DNA from selected colonies is isolated and subjected to DNA sequencing to confirm the presence of the insert.

Transformed host cells are incubated in 2xYT medium containing 30 mg/ml kanamycin at 30° C. prior to induction. Gene expression is induced by the addition of N-(3-oxohexanoyl)-dl-homoserine lactone to a final concentration of 30 ng/ml followed by incubation at either 30° C. or 37° C. for six hours. The expression of AFTI polypeptide is evaluated by centrifugation of the culture, resuspension and lysis of the bacterial pellets, and analysis of host cell proteins by SDS-polyacrylamide gel electrophoresis.

Inclusion bodies containing AFTI polypeptide are purified as follows. Bacterial cells are pelleted by centrifugation and resuspended in water. The cell suspension is lysed by sonication and pelleted by centrifugation at 195,000×g for 5 to 10 minutes. The supernatant is discarded, and the pellet is washed and transferred to a homogenizer. The pellet is homogenized in 5 ml of a Percoll solution (75% liquid Percoll. 0.15M NaCl) until uniformly suspended and then is diluted and centrifuged at 21,600×g for 30 minutes. Gradient fractions containing the inclusion bodies are recovered and pooled. The isolated inclusion bodies are analyzed by SDS-PAGE.

A single band on an SDS polyacrylamide gel corresponding to E. coli.-produced AFTI polypeptide is excised from the gel, and the N-terminal amino acid sequence is determined essentially as described by Matsudaira et al., J. Biol. Chem., 262: 10-35 (1987).

B. Mammalian Cell Production

PCR is used to amplify template DNA sequences encoding an AFTI polypeptide using primers corresponding to the 5′ and 3′ ends of the sequence. The amplified DNA products may be modified to contain restriction enzyme sites to allow for insertion into expression vectors. PCR products are gel purified and inserted into expression vectors using standard recombinant DNA methodology. An exemplary expression vector, pCEP4 (Invitrogen, Carlsbad, Calif.), which contains an Epstein-Barr virus origin of replication, may be used for the expression of AFTI in 293-EBNA-1 (Epstein-Barr virus nuclear antigen) cells. Amplified and gel purified PCR products are ligated into pCEP4 vector and lipofected into 293-EBNA cells. The transfected cells are selected in 100 mg/ml hygromycin and the resulting drug-resistant cultures are grown to confluence. The cells are then cultured in serum-free media for 72 hours. The conditioned media is removed and, AFTI polypeptide expression is analyzed by SDS-PAGE.

AFTI polypeptide expression may be detected by silver staining. Alternatively, AFTI polypeptide is produced as a fusion protein with an epitope tag, such as an IgG constant domain or a FLAG epitope, which may be detected by Western blot analysis using antibodies to the tag peptide.

AFTI polypeptides may be excised from an SDS-polyacrylamide gel, or AFTI fusion proteins are purified by affinity chromatography to the epitope tag, and are subjected to N-terminal amino acid sequence analysis as described herein.

### EXAMPLE 3

### Production Of Anti-AFTI Polypeptide Antibodies

Antibodies to AFTI polypeptides may be obtained by immunization with purified protein or with AFTI peptides produced by biological or chemical synthesis. Suitable procedures for generating antibodies include those described in Hudson and Hay, Practical Immunology, 2nd Edition, Blackwell Scientific Publications (1980).

In one procedure for the production of antibodies, animals (typically mice or rabbits) are injected with an AFTI antigen (such as an AFTI polypeptide), and those with sufficient serum titer levels as determined by ELISA are selected for hybridoma production. Spleens of immunized animals are collected and prepared as single cell suspensions from which splenocytes are recovered. The splenocytes are fused to mouse myeloma cells (such as Sp2/0-Ag14 cells; ATCC no. CRL-1581), are allowed to incubate in DMEM with 200 IU/ml penicillin, 200 mg/ml streptomycin sulfate, and 4 mM L-glutamine, then are incubated in HAT selection medium (Hypoxanthine; Aminopterin; Thymidine). After selection, the tissue culture supematants are taken from each fusion well and tested for anti-AFTI antibody production by ELISA.

Alternative procedures for obtaining anti-AFTI antibodies may also be employed, such as the immunization of transgenic mice harboring human Ig loci for the production of human antibodies, and the screening of synthetic antibody libraries, such as those generated by mutagenesis of an antibody variable domain.

While the present invention has been described in terms of the preferred embodiments, it is understood that variations and modifications will occur to those skilled in the art. Therefore, it is intended that the appended claims cover all such equivalent variations that come within the scope of the invention as claimed.

1-49. (canceled)

50. A method for reducing inflammation in a subject comprising administering to said subject an effective amount of an apolipoprotein polypeptide selected from the group consisting of:
(a) Apo-A-I:
(b) Apo-A-II;
(c) a polypeptide comprising an amino acid sequence as set forth in residues 25 to 194 of SEQ ID NO:2:
(d) a polypeptide comprising an amino acid sequence as set forth in residues 25 to 144 of SEQ ID NO:2:
(e) a polypeptide comprising an amino acid sequence as set forth in residues 156 to 267 of SEQ ID NO:2:
(f) a polypeptide comprising an amino acid sequence as set forth in residues 25 to 113 of SEQ ID NO:2:
(g) a polypeptide comprising an amino acid sequence as set forth in residues 75 to 113 of SEQ ID NO:2: and
(h) a polypeptide comprising an amino acid sequence that is at least about 95 percent identical to the amino acid sequence of at least one of (a) through (g) wherein the polypeptide has an activity of the polypeptide as set forth in SEQ ID NO:2.

51-61. (canceled)

62. The method of claim 50, wherein the polypeptide is Apo-A-I.

63. The method of claim 50, wherein the polypeptide is Apo-A-II.

64. The method of claim 50, wherein the polypeptide comprises an amino acid sequence as set forth in residues 25 to 194 of SEQ ID NO:2.

65. The method of claim 50, wherein the polypeptide comprises an amino acid sequence as set forth in residues 25 to 144 of SEQ ID NO:2.

66. The method of claim 50, wherein the polypeptide comprises an amino acid sequence as set forth in residues 156 to 267 of SEQ ID NO:2.

67. The method of claim 50, wherein the polypeptide comprises an amino acid sequence as set forth in residues 25 to 113 of SEQ ID NO:2.

68. The method of claim 50, wherein the polypeptide comprises an amino acid sequence as set forth in residues 75 to 113 of SEQ ID NO:2.

69. The method of claim 50, wherein the polypeptide comprises an amino acid sequence that is at least about 95 percent identical to the amino acid sequence of at least one of (a) through (g), wherein the polypeptide has an activity of the polypeptide as set forth in SEQ ID NO:2.

70. The method of claim 50, wherein said polypeptide reduces IL-113 secretion in the subject.

71. The method of claim 50, wherein said polypeptide reduces TNF-α secretion in the subject.

72. The method of claim 50, wherein said polypeptide reduces monocyte activation in the subject.
